(19) United States
(12) Patent Application Publication
(10) Pub. No.: US 2023/0033797 A1
(43) Pub. Date: Feb. 2, 2023

(54) UNIVERSAL PRECURSOR FOR NANOSCALE MORPHOLOGIES

(71) Applicant: NewSouth Innovations Pty Limited, Sydney (AU)

(72) Inventors: Sajjad Seifi MOFARAH, Sydney (AU); Charles Christopher SORRELL, Sydney (AU); Pramod KOSHY, Sydney (AU)

(21) Appl. No.: 17/755,247

(22) PCT Filed: Oct. 26, 2020
(86) PCT No.: PCT/AU2020/051153

§ 371 Date: Apr. 25, 2022

(30) Foreign Application Priority Data
Oct. 25, 2019 (AU) 2019904036

(51) Int. Cl. C08G 83/00; B82Y 30/00; B82Y 40/00; C07C 51/41; B01J 20/22

(52) U.S. Cl. CPC C08G 83/001; B82Y 30/00; B82Y 40/00; C07C 51/418; B01J 20/226; C08G 83/008

(57) ABSTRACT

A metal coordination polymer, in particular, a layered metal coordination polymer, can be used as a precursor to form nanostructures of various morphologies and composition. Metal based nanostructures can be prepared from the metal coordination polymers. The nanostructures may have various catalytic properties. The layered metal coordination polymer includes two or more layers, each layer including metal atoms coordinated to an organic linker to form a metal coordination polymer layer.

### CROSS-REFERENCE

The present application claims priority from Australian Provisional Patent Application No. 2019904036 filed on 25 Oct. 2019, the entire contents of which are incorporated herein by reference.

### FIELD

The present disclosure relates to a metal coordination polymer. In particular, the present disclosure relates to a layered metal coordination polymer, which can be used as a precursor to form nanostructures of various morphologies and composition. The present disclosure also relates to metal based nanostructures, which can be prepared from the metal coordination polymers.

### BACKGROUND

2D structures (e.g., sheets including nanosheets) have established new levels of functionalities for materials, particularly for energy and environmental applications. Minimisation of transverse charge carrier diffusion distance is achieved by reducing sheet thickness. However, reducing a sheet thickness can only go so far as the structure and chemistry of the sheet can dictate sheet thickness.

Another way to minimise charge carrier diffusion is to reduce a lateral distance of the sheet, for example by the introduction of holes. The formation of holes in nanosheets enhances the density of accessible active sites and shortens the distance of lateral charge carrier diffusion. However, to minimise the transverse diffusion distances within holey 2D materials, sheets with thickness in the atomic range should be achieved. Additionally, to retain highly active sheets, polycrystalline 2D planar materials are desirable to prevent irreversible restacking of the nanosheets. However, the synthesis of polycrystalline holey 2D sheets by either top-down or bottom up strategies has remained elusive for most compounds.

The fabrication of holey 2D graphene and holey 2D transition metal chalcogenides (TMCs) and selenides (TMS) have been reported. However, the processing is relatively complex, and requires surfactants, sacrificial templates, and/or additional steps for removal of the template at high temperatures, which ultimately result in nanosheet thicknesses of tens of nanometres. However, to date there is little information in terms of effective synthesis of holey 2D metal oxides (MOs).

It is to be understood that, if any prior art publication is referred to herein, such reference does not constitute an admission that the publication forms a part of the common general knowledge in the art, in Australia or any other country.

### SUMMARY

The present inventors have undertaken research and development into metal coordination polymers that can be used to make a variety of metal based nanostructures, including holey metal oxide nanosheets. In particular, the metal coordination polymers as described herein are inherently unstable and comprise reactive metal centres, which can be stabilised by the presence of one or more organic linkers. When used as a precursor, the removal of the organic linkers generates a highly reactive metal-based substructure, which can then subsequently form various stable nanostructures, allowing for a unique and tailored process to prepare nanostructures with varied morphologies.

The metal coordination polymer may be a layered metal coordination polymer. The layered metal coordination polymer may comprise two or more layers. The layers of the metal coordination polymer comprise metal atoms each coordinated to one or more organic linkers to form a metal coordination polymer layer. Two or more of these metal coordination polymer layers may electrostatically interact to form the layered metal coordination polymer. An electrostatic interaction may form between the metal coordination polymer layers. The organic linker comprises a metal binding moiety, which can form coordinative bonding to a metal atom to form the metal coordination polymer layer. The organic linker also comprises one or more moieties for forming an electrostatic interaction with an adjacent metal coordination polymer layer to form the layered metal coordination polymer. The one or more moieties may be substituted on an optionally interrupted alkyl, alkenyl or alkynyl group and/or may be substituted directly onto the metal binding moiety. The metal coordination polymer may also be a reaction product of an organic linker and a source of metal atoms.

In one aspect, there is provided a layered metal coordination polymer comprising two or more layers, each layer comprising metal atoms coordinated to an organic linker to form a metal coordination polymer layer;

wherein the organic linker is selected from one or more compounds having the structure of Formula 1:

X—R¹ (1)

wherein:

X is a metal binding moiety for coordinative bonding to a metal atom; and

R¹ is H or an optionally interrupted alkyl, alkenyl or alkynyl group substituted with one or more moieties for forming an electrostatic interaction with an adjacent metal coordination polymer layer to form the layered metal coordination polymer.

In another aspect, there is provided a layered metal coordination polymer which is the reaction product of an organic linker and a source of metal atoms, the layered metal coordination polymer comprising two or more layers, each layer comprising metal atoms each coordinated to one or more organic linkers to form a metal coordination polymer layer;

wherein the organic linker is a compound having the structure of Formula 1:

X—R¹ (1)

wherein:

X is a metal binding moiety for coordinative bonding to a metal ion; and

R¹ is H or an optionally interrupted alkyl, alkenyl or alkynyl group substituted with one or more moieties for forming an electrostatic interaction with an adjacent metal coordination polymer layer to form the layered metal coordination polymer.

In another aspect, there is provided a process for preparing a layered metal coordination polymer comprising two or more metal coordination polymer layers as defined above, comprising:

combining a metal atom source and an organic linker to form a layered metal coordination polymer comprising two or more metal coordination polymer layers that are held together by an electrostatic interaction.

In another aspect, there is provided a process for preparing a layered metal coordination polymer comprising two or more metal coordination polymer layers as defined above, comprising:

mixing an aqueous solution comprising a metal atom source and an organic linker to form a layered metal coordination polymer comprising two or more metal coordination polymer layers that are held together by an electrostatic interaction.

In another aspect, there is provided a method of forming a nanostructure, comprising:

providing a layered metal coordination polymer comprising two or more layers, each layer comprising metal atoms each coordinated to one or more organic linkers to form a metal coordination polymer, and

removing at least some of the coordinating organic linkers to form the nanostructure.

In another aspect, there is provided a nanostructure prepared using the method as defined above.

In another aspect, there is provided a holey metal oxide nanosheet. The holey metal oxide nanosheet may have a thickness of between about 1 nm to about 100 nm.

In another aspect, there is provided a catalyst composition comprising the nanostructure defined above.

In another aspect, there is provided use of the nanostructure defined above as a catalyst.

It will be appreciated that any one or more of the embodiments and examples described herein for the metal coordination polymer may also apply to the process for preparing the metal coordination polymer, the method for preparing a nanostructure described herein, the nanostructure described herein and/or the catalyst composition described herein. Any embodiment herein shall be taken to apply mutatis mutandis to any other embodiment unless specifically stated. It will also be appreciated that other aspects, embodiments and examples of the metal coordination polymer and nanostructure are described herein.

It will also be appreciated that some features of the metal coordination polymer, process for preparing the metal coordination polymer, nanostructures, and method for preparing the nanostructures identified in some aspects, embodiments or examples as described herein may not be required in all aspects, embodiments or examples as described herein, and this specification is to be read in this context. It will also be appreciated that in the various aspects, embodiments or examples, the order of method or process steps may not be essential and may be varied.

### BRIEF DESCRIPTION OF FIGURES

Embodiments of the present disclosure are further described and illustrated as follows, by way of example only, with reference to the accompanying drawings where applicable, in which:

FIG. 1 shows exfoliation and conversion of Ce-coordination polymer (CP) nanotube into holey 2D CeO₂₋ₓ nanostructures: a-c) Ex-situ SEM, TEM, schematic of Ce-CP hexagonal; d-f) Ex-situ SEM, TEM, schematic of Ce-CP nanosheet obtained by exfoliation of the Ce-CP hexagonal nanotube for 4 min at room temperature; g-i) Ex-situ SEM, TEM, schematic of Ce-CP nanosheet obtained by exfoliation of the Ce-CP hexagonal nanotube for 8 min at room temperature; j-l) Ex-situ SEM, TEM, schematic of holey CeO₂₋ₓ nanosheet obtained by exfoliation of the Ce-CP hexagonal nanotube for 15 min at room temperature in basic aqueous solution (pH=8); m) Schematic of layered structure of Ce-CP; n) Ce-CP exfoliated nanosheets as a result of penetration of water molecules between stacked Ce-CP nanosheets; and o) highly defective CeO₂₋ₓ nanosheets. Blue, green, red, brown, and black spheres represent cerium, chlorine, oxygen, carbon, and hydrogen ions, respectively. Gaps within the defective CeO₂₋ₓ nanosheet represent oxygen vacancies.

FIG. 1A shows schematic of stratified (layered) structure of Ce-CP. Large yellow spheres=Ce⁴⁺, small green spheres=C⁴⁺, small black sphere=H⁺, small blue spheres=O²⁻, small red spheres=Cl⁻.

FIG. 2 shows defect and structural analysis of CeO₂₋ₓ holey nanosheets: a,b) Low-magnification HAADF image of CeO₂₋ₓ nanosheet; c) High-magnification HAADF image of CeO₂₋ₓ nanosheets illustrating nanoholes of ˜2-5 nm lateral size; d) EELS spectra from an intercrystallite region in CeO₂₋ₓ nanosheets; e) EELS spectra from within a CeO₂₋ₓ crystallite; and f) High-magnification HAADF image showing Ce vacancies within a CeO₂₋ₓ crystallite.

FIG. 3 shows characterisation of holey metal oxide (MO) nanosheets: TEM image for a) CeO₂₋ₓ nanosheet, b) TiO₂₋ₓ nanosheet, c) ZrO₂₋ₓ nanosheet; corresponding SAED patterns of d) CeO₂₋ₓ nanosheet, e) TiO₂₋ₓ nanosheet, f) ZrO₂₋ₓ nanosheet; AFM image of g) CeO₂₋ₓ nanosheet, h) TiO₂₋ₓ nanosheet, i) ZrO₂₋ₓ nanosheet; and corresponding height profiles for j) CeO₂₋ₓ nanosheet, k) TiO₂₋ₓ nanosheet, 1) ZrO₂₋ₓ nanosheet.

FIG. 4 shows characterisation of transition metal oxide (TMO) in 0D/2D heterostructures: a-c) EDS mapping of Fe₂O₃-functionalised CeO₂₋ₓ nanosheet (FCO), NiO-functionalised CeO₂₋ₓ nanosheet (NCO), and ZnO-functionalised CeO₂₋ₓ nanosheet (ZCO) 0D/2D heterostructures, respectively; d-f) Laser Raman microspectra of FCO, NCO, and ZCO 0D/2D heterostructures, respectively; and g-i) XRD patterns of FCO, NCO, and ZCO 0D/2D heterostructures, respectively.

FIG. 5 shows band structure characterisation of CeO₂₋ₓ and 0D/2D heterostructures: a) Topography of CeO₂₋ₓ holey nanosheet; b) Contact potential difference measured by KPFM of CeO₂₋ₓ holey nanosheet; c) XPS valence band plot for CeO₂₋ₓ holey nanosheet; d) Tauc plot from UV-Vis spectrophotometry data for CeO₂₋ₓ holey nanosheet (Tauc plot model (αhυ)=A(hυ−E_{g})² applied, where A and α are absorption and absorption coefficient, respectively; hu is photon energy, and E_{g} is optical indirect band gap); e) Electronic energy level diagram for CeO₂₋ₓ holey nanosheet and 0D/2D heterostructures; f) First-principles DFT computations of electronic densities of states and band gaps of CeO₂ nanosheets and bulk CeO₂; and g-i) First-principles DFT computations of electronic densities of states and band gaps of 0D/2D heterostructures.

FIG. 6 shows formation mechanism of Ce-CP tubes under constant current electrochemical deposition. a) current-deposition time plot; b-f) SEM images representing the nucleation/growth process of the Ce-CPs tube as a function of electrodeposition time.

FIG. 7 shows Pourbaix diagram demonstrating thermodynamic study on the quaternary aqueous system Ce(III)-Ce(IV)-trichloroacetic acid (TCA)-H₂O as a function of pH.

FIG. 8 shows experimental X-ray diffraction pattern of Ce-CP.

FIG. 9 shows neutron diffraction pattern of Ce-CP obtained at wavelengths of 1.63 Å and 2.41 Å.

FIG. 10 shows SEM images of Ce-CP tubes grown on FTO substrate.

FIG. 10A shows a) the low magnification TEM image of a single Ce-CP tube. b) SAED pattern of region shown in the yellow box and c) HRTEM image of region shown in the red box.

FIG. 11 shows Raman spectra of Ce-CP tube (top) and trichloroacetic acid (bottom).

FIG. 11A shows Raman spectra of CeO₂ (top), Ce-CP tube (middle) and trichloroacetic acid (bottom).

FIG. 12 shows FTIR spectra of Ce-CP tubes.

FIG. 13 shows XPS data of Ce-CP tubes.

FIG. 14 shows TGA analysis of Ce-CP in nitrogen (top) and air (bottom) atmospheres.

FIG. 15 shows Rietveld-refined X-ray diffraction pattern of Ce-CP.

FIG. 16 shows Rietveld-refined ND patterns of Ce-CP at wavelengths of 1.63 Å (bottom) and 2.41 Å (top).

FIG. 17 shows schematic of refined structure from XRD and ND data.

FIG. 18A shows the relaxed structure of the smallest possible Ce-CP unit cell used as the building block for constructing a more representative structure model.

FIG. 18B shows: (a) The relaxed Ce-CP structure commensurate with experimental lattice parameters. All TCA molecules were found to remain intact. A, B, and C denote the Ce ion bonding to a TCA molecule, a water molecule, and an OH group respectively. (b-e) The site projected partial density of states of the marked Ce ion and the O ions from distinct coordinating ligands.

FIG. 19 Comparison of X-ray diffraction patterns of experimental, Rietveld refined, and ab initio MD simulated structures.

FIG. 20 shows structural and morphological evolution of Ce-CP hexagonal nanotube into CeO₂₋ₓ nanosheets: a) SEM image of Ce-CP hexagonal nanotube; b) TEM image of Ce-CP hexagonal nanotube (inset: SAED pattern); c) XRD pattern of Ce-CP hexagonal nanotube; d) SEM image of Ce-CP nanosheet; e) TEM image of Ce-CP nanosheet (inset: SAED pattern); f) XRD pattern of Ce-CP nanosheet; g) SEM image of holey CeO₂₋ₓ nanosheet; h) TEM image of holey CeO₂₋ₓ nanosheet (inset: SAED pattern); i) XRD pattern of CeO₂₋ₓ nanosheet.

FIG. 21 shows a) TEM image of Ce-CP nanosheet. EDS elemental mapping image of b) cerium (red), c) oxygen (green), d) chlorine (navy blue), e) carbon (light blue). f) EDS spectra of Ce-CP nanosheet.

FIG. 22 shows a,b) Bright field TEM image of CeO₂₋ₓ holey nanosheets. EDS elemental mapping image of c) oxygen (green), d) cerium (red), e) EDS spectra of CeO₂₋ₓ holey nanosheets.

FIG. 23 shows Raman spectrum of CeO₂₋ₓ nanosheets compared with that of original Ce-CP, indicating insignificant differences.

FIG. 24 shows SEM images of Ti-CP.

FIG. 25 shows SEM image of Ti-CP. EDS elemental mapping images of b) titanium, c) oxygen and d) carbon, e) overlay of EDS images of Ti-CP, f) corresponding EDS spectra.

FIG. 26 shows Raman spectra of Ti-CP.

FIG. 27 shows SEM images of Zr-CP.

FIG. 28 shows a) SEM image of Zr-CP, EDS elemental mapping images of b) zirconium, c) oxygen, d) carbon, e) overlay of EDS images of Zr-CP, f) corresponding EDS spectra.

FIG. 29 shows Raman spectra of Zr-CP.

FIG. 30 shows a-c) TEM images of ultrathin Ti-CP nanosheets exfoliating in DI water at room temperature.

FIG. 31 shows a-c) TEM images of ultrathin holey TiO₂ nanosheet along with d) corresponding SAED pattern of TiO₂ nanosheets.

FIG. 32 shows Raman spectrum of TiO₂ nanosheets (black) and corresponding fits for vibrational modes of anatase (blue) and rutile (red) phases.

FIG. 33 shows XPS results for is orbital of carbon in both Ti-CP and TiO₂ nanostructure.

FIG. 34 shows XPS results for is orbital of oxygen in both Ti-CP and TiO₂ nanostructure.

FIG. 35 shows TEM images illustrating exfoliation of bulk Zr-CP in DI water at room temperature and formation of free-standing Zr-CP nanosheets.

FIG. 36 shows a-c) TEM images of ultrathin holey ZrO₂ nanosheets obtained by exfoliation of Zr-CP in DI water at room temperature. d) SAED pattern of ZrO₂ nanosheet revealing the polycrystalline nature of nanosheets.

FIG. 37 shows Raman spectrum of zirconium oxide nanosheets (black) and corresponding fits for vibrational modes of monoclinic (blue) and cubic (red) phases.

FIG. 38 shows XPS results for is orbital of carbon in both Zr-CP and ZrO₂ nanostructure.

FIG. 39 shows XPS results for is orbital of carbon in both Zr-CP and ZrO₂ nanostructure.

FIG. 40 shows XPS results for 3d orbital of cerium and is orbital of oxygen in CeO₂₋ₓ holey nanostructure.

FIG. 41 shows zeta potentials of CeO₂₋ₓ in DI water.

FIG. 42 shows speciation diagrams for a) Fe (II), b) Ni (II), c) Zn (II) species representing stability of the species and concentration variations of species as a function of pH in aqueous solution.

FIG. 43 shows TEM and HRTEM images of FCO; d) SAED pattern of FCO. e-g) TEM and HRTEM images of NCO. h) SAED pattern of NCO. i-k) TEM and HRTEM images of ZCO. 1) SAED pattern of ZCO.

FIG. 44 shows XPS valence measurement of a) holey CeO₂₋ₓ nanosheet, b) FCO, c) NCO, d) ZCO.

FIG. 45 shows Tauc plot for a) holey CeO₂₋ₓ nanosheet, b) FCO, c) NCO, d) ZCO.

FIG. 46 shows photoluminescence spectra of CeO₂₋ₓ, FCO, NCO, ZCO.

FIG. 47 shows: a) methylene blue (MB) degradation in the presence of holey nanosheet (blue bar) and NiO (purple) and Fe₂O₃ (green) anchored holey nanosheet; b) the kinetics of the MB degradation; c) Comparison table from the as-synthesised samples and recently reported results for MB degradation; d) summary of MB degradation performances of CeO₂₋ₓ structures.

FIG. 48 shows TEM and SEM micrographs of CeO₂₋ₓ nanostructures derived from Ce-CP (scale bar yellow=3 μm, red=100 nm). For f and h, low-magnification TEM images rather than SEM images are shown owing to the small sizes of the cubic and dumbbell-like morphologies, respectively.

FIG. 49 shows a schematic illustration of a) Three-electrode electrochemical cell used for the synthesis of Ce-CP tubes under vigorous oxygen evolution and deposition of free-standing hexagonal tubes of Ce-CP on fluorine-doped tin oxide (FTO) substrate, b) CeO₂₋ₓ formation through the three-step process including exfoliation of the Ce-CP tube into Ce-CP nanosheets and subsequently oxidation of Ce-CP nanosheet into holey CeO₂₋ₓ nanosheet.

FIG. 50 shows schematics of a) chronopotentiometric electrodeposition of solid Ce-CP hexagonal rods under electrolysis conditions; b) dissolution of Ce-CP hexagonal rods and recrystallisation of Ce-CP into hollow pseudo-octahedra. c) Simplified molecular structures of hexagonal Ce-CP rod, d) schematic of solutes in ethanol solution e) corresponding molecular structure, f) schematic of recrystallised Ce-CP, g) corresponding molecular structure. Large yellow spheres=Ce⁴⁺, small green spheres=C⁴⁺, small blue spheres=O₂, small red spheres=Cl⁻.

FIG. 51 shows (a,b) SEM and (c,d) TEM images of Ce-CP structures (inset shows respective SAED pattern).

FIG. 52 shows experimental X-ray diffraction patterns obtained from a) freshly prepared Ce-CP and (b) aged sample (under ambient condition) for 3 months.

FIG. 53 shows a schematic of: (a) Formation of Ce-CP monolayer at ethanol/air interface: Ce⁴⁺ (green), —OH group of ethanol (purple), —COO— group of TCA (blue), and —CCl₃ group of TCA (red), b) Monolayer and stacking arrangement (residual —OH and H₂O are omitted from Ce—CP and solution volume for simplicity), c) Optical microscopy image of Ce-CP nanosheets, d) AFM image of Ce-CP nanosheet and index corresponding to height profile, e) A low magnification TEM image of Ce-CP nanosheets; inset: SAED pattern of Ce-CP nanosheet, f-k) EDS mapping of the Ce-CP nanosheet showing maps for (g) Ce; h) O; i) Cl; j) C; k) Sn.

FIG. 54 shows AFM image and corresponding height profile of Ce-CP nanosheets printed from surface of ethanol at evaporation times of a) 12 h, b) 24 h c) 36 h d) 48 h, (e) 72 hours.

FIG. 55 shows AFM image and corresponding height profile of Ce-CP nanosheets printed from surface of ethanol at different Ce-CP concentrations of a,b) 4 M, c,d) 8 M.

FIG. 56 shows a,b) HAADF images and (b, inset) SAED image of the holey CeO₂₋ₓ nanosheet, c) HRTEM image of the holey CeO₂₋ₓ nanosheet, d) XPS spectra of Ce 3d orbital of Ce in holey CeO₂₋ₓ nanosheet, e) AFM image of holey CeO₂₋ₓ nanosheet, f) AFM height profile of CeO₂₋ₓ nanosheet.

FIG. 57 shows a) SEM image, b) Schematic of as-recrystallised Ce-CP c) corresponding XRD pattern, d) SEM image, e) schematic of NaOH-aged CeO₂₋ₓ pseudo-octahedron, f) Corresponding XRD pattern, g) SEM image, h) schematic of CeO₂₋ₓ pseudo-octahedron, i) Corresponding XRD pattern, j) Dark field TEM and SAED (inset), k) Dark field HRTEM image of CeO₂₋ₓ pseudo-octahedron.

FIG. 58 shows (a) XRD patterns of Ce-CP rod synthesised by electrochemical deposition (black) and Ce-CP octahedron obtained by dissolution/recrystallisation method in ethanol (red), b) HRTEM image of Ce-CP rod (left) and Ce-CP octahedron (enclosed regions by yellow solid line show single crystallites, c) Raman spectra of Ce-CP rod (black) and Ce-CP octahedron (red), d) FTIR spectra of Ce-CP rod (black) and Ce-CP octahedron (red).

FIG. 59 shows SEM images of Ce-CP morphologies synthesised at 0° C.: a) [Ce-CP]=4 M, b) [Ce-CP]=16 M, c) hollow spheres being liberated from nanosheets, d) Schematic showing the formation of Ce-CP hollow spheres through bubbling of the stacked nanosheets as a result of ethanol evaporation, e) 3D AFM image of the Ce-CP nanosheets synthesised by two-stage evaporation at −10° C. (12 h) and 15° C. (0.5 h), f) AFM height profile (black dotted line).

FIG. 60 shows characterisation of hollow CeO₂₋ₓ spheres: a) Low-magnification and b) high-magnification SEM images of hollow CeO₂₋ₓ spheres, c) SEM image of broken hollow spheres, d) Low magnification TEM image of the hollow CeO₂₋ₓ spheres, e,f) High-magnification TEM image of the hollow CeO₂₋ₓ spheres, g) SAED pattern of the hollow CeO₂₋ₓ spheres, h,i) EDS elemental mapping of Ce and O in the hollow CeO₂₋ₓ spheres, j) Raman spectra of Ce-CP rods before and after NaOH ageing and heating at 200° C.

FIG. 61 shows SEM images of the Ce-CP nanostructures synthesised at 25° C. using varying Ce-CP concentrations of a) 4 M, b) 8 M, c) 40 M, d) 120 M e-h) SEM images of the corresponding CeO₂₋ₓ nanostructures derived from the Ce-CP by aging in NaOH (6 M) at 25° C. followed by subsequent heating at 200° C.

FIG. 62 shows SEM, TEM, HRTEM images and SAED pattern of CeO₂₋ₓ derived from Ce-CP morphologies synthesised at 25° C.: a-c) 5 mM, d-f) 10 mM, g-i) 50 mM, j-l) 100 mM.

FIG. 63 shows formation mechanism for the Ce-CP nanostructures.

FIG. 64 shows a) CO conversion rate and TOF values for CO oxidation obtained by using different nanostructured morphologies of CeO₂₋ₓ, b) Arrhenius plots for the oxidation of CO over the samples.

FIG. 65 shows XPS Ce 3d spectra for holey nanosheet, hollow octahedron, hollow sphere, and leaf CeO₂₋ₓ.

FIG. 66 shows XPS Ce 3d spectra for holey nanosheet, hollow octahedron, hollow sphere, and leaf CeO₂₋ₓ.

FIG. 67 shows photocatalytic performances of the CeO₂₋ₓ morphologies: a) UV-Vis absorption spectra of MB dye solution following 160 min irradiation for different morphologies, (b) 664 nm peak intensities based on UV-Vis absorption spectra of MB dye solution at different irradiation times for different morphologies, c) plots of absorbance (Aₜ/A₀, at time t vs initial time) and extent of the dye degradation as a function of irradiation time for holey nanosheet, d) comparison of the photocatalytic performances obtained in this work and that of prior art under similar testing conditions.

FIG. 68 shows effects of structural and physical properties of the morphologies on their catalytic and photocatalytic performances.

FIG. 69 shows the zeta potential of the layered Ce-CP in DI water.

FIG. 70 shows structural analysis a) XRD spectra and b) laser Raman microspectra of Ce-CP nanotubes, reassembled Ce-CP macrolayers (DMSO-derived, R—Ce—CP), air-calcined Ce/S/C, N₂-calcined Ce/S/C (all intensities scaled identically).

FIG. 71 shows XPS spectra of a) Cl 2p, b) C Is, c) S 2p orbitals of Ce-CP nanotubes (NT), DMSO-derived Ce-CP (Tᵣₒₒₘ), air-calcined Ce/S/C (air), N₂-calcined Ce/S/C (N₂).

FIG. 72 shows XPS spectra for (a) Ce 3d orbital and (b) O 1s orbital for Ce-CP, DMSO-derived Ce-CP, air-calcined Ce/S/C, N₂ calcined Ce/S/C samples.

FIG. 73 shows EPR analysis of Ce/S/C and pristine CeO₂.

FIG. 74 shows XRD pattern of polycrystalline octahedral nanostructure compared with that of original Ce-CP.

FIG. 75 shows a) HAADF-STEM images and EELS-STEM maps for the Ce—O—S sample. The maps have been obtained by extracting S K-edge signal at 165 eV (green), C K-edge signal at 284 eV (yellow), O K-edge at 532 eV (blue), and Ce M-edge at 883 eV (red), b) Ce M₅/M₄ ratio to evaluate the cerium oxidation state distribution, the color legend is reported as well, c) normalised EELS spectra for C K-edge peak and d) Ce M-edge peaks. Scale bar is 100 nm.

FIG. 76 shows HRTEM images from the sample together with the corresponding indexed power spectrum and the frequency-filtered map highlighting different crystals.

FIG. 77 shows a) Schematic of two-step process of Ce-CP nanotube exfoliation in stirred TEA solution and oxidation at 450° C. in air into stacked CeO₂₋ₓ macrolayers, b-e) CeO₂₋ₓ morphologies derived from Ce-CP.

FIG. 78 SEM images of CeO₂₋ₓ obtained at 450° C. at different heating rates: a) low-rate calcination at 0.2° C. min⁻¹, b) medium-rate calcination at 1.0° C. min⁻¹, c) high-rate calcination at 2.0° C. min¹, d) high-rate calcination at 3.0° C. min⁻¹.

FIG. 79 shows a-c) SEM images of hybrid 2D-3D CeO₂₋ₓ, d) HRTEM image and SAED of holey 2D CeO₂₋ₓ nanosheet (holes outlined), e) EDS elemental mapping of holey 2D CeO₂₋ₓ nanosheet f) AFM image (step height in while dotted line) and corresponding height profile of holey 2D CeO₂₋ₓ nanosheet.

FIG. 80 shows a) TEM and b) HRTEM images of holey Mn—Ce nanosheet, c) SAED pattern of holey Mn—Ce nanosheet, d) STEM elemental mapping of O, Mn, Ce in holey Mn—Ce nanosheet, e) STEM line scan across the holey Mn—Ce nanosheet.

FIG. 81 shows XRD spectra of 2D-3D CeO₂₋ₓ, Mn—Ce, Cu—Ce (α-MnO₂ indicated by Miller indices in Mn—Ce).

FIG. 82 shows a) CO oxidation plots for Ce-NT, CeO₂₋ₓ, Mn—Ce, Cu—Ce, b) Comparative CO oxidation data for CeO₂₋ₓ and CeO₂₋ₓ-based hybrids, c) Mechanism 1: CO-oxidation reaction path with initial O₂ adsorption deduced from first-principles calculations based on DFT, d) Energy profiles calculated for Mechanism 1, e) Mechanism 2: CO-oxidation reaction path with initial CO adsorption deduced from first-principles DFT calculations, f) Energy profiles calculated for Mechanism 2.

### DETAILED DESCRIPTION

### General Terms

In the following description, reference is made to the accompanying drawings, which form a part hereof, and which is shown, by way of illustration, several embodiments. It is understood that other embodiments may be utilised and structural changes may be made without departing from the scope of the present disclosure.

With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. For example, all technical and scientific terms used herein shall be taken to have the same meaning as commonly understood by one of ordinary skill in the art (for example, in materials science, inorganic chemistry, polymer chemistry, and nanotechnology etc.). The definitions are provided to aid in describing particular embodiments, and are not intended to limit the claimed invention, because the scope of the invention is limited only by the claims. Furthermore, unless otherwise required by context, singular terms shall include pluralities and plural terms shall include the singular.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, coatings, processes, and coated substrates, referred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings or for either meaning. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Unless otherwise indicated, the terms “first,” “second,” “further” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the term “about”, unless stated to the contrary, typically refers to +/−10%, for example +/−5%, of the designated value.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “consists of”, or variations such as “consisting of”, refers to the inclusion of any stated element, integer or step, or group of elements, integers or steps, that are recited in context with this term, and excludes any other element, integer or step, or group of elements, integers or steps, that are not recited in context with this term.

### Specific Terms

The following definitions apply to the terms as used throughout this specification, unless otherwise limited in specific instances.

The term “organic linker” refers to a compound capable of forming one or more coordinative bonds to one or more metal atoms.

The term “metal binding moiety” refers to chemical moiety capable of coordinating (e.g., bonding) to a metal. Non-limiting examples of a metal binding ligand moiety include —COOH, —OH, —NH₂, —SH, and —CN.

The term “optionally substituted” means that a functional group is either substituted or unsubstituted, at any available position. It will be appreciated that “unsubstituted” refers to a hydrogen group. Substitution can be with one or more functional groups selected from one or more heteroatom, including one or more O, N, S, Se, Te, Si, and/or one or more alkenyl, alkynyl, aryl, heteroaryl, heteroaryl, and/or cycloalkyl, in which each alkenyl, alkynyl, aryl, heteroaryl, or cycloalkyl group, which alkenyl, alkynyl, aryl, heteroaryl, heteroaryl, and/or cycloalkyl, in which each alkenyl, alkynyl, aryl, heteroaryl, or cycloalkyl group is as defined herein.

“Alkyl” whether used alone, or in compound words such as alkoxy, alkylthio, alkylamino, dialkylamino or haloalkyl, represents straight or branched chain hydrocarbons ranging in size from one to about 20 carbon atoms, or more. Thus alkyl moieties include, unless explicitly limited to smaller groups, moieties ranging in size, for example, from one to about 6 carbon atoms or greater, such as, methyl, ethyl, n-propyl, iso-propyl and/or butyl, pentyl, hexyl, and higher isomers, including, e.g., those straight or branched chain hydrocarbons ranging in size from about 6 to about 20 carbon atoms, or greater. “C₁₋₂₀alkyl”, “C₁₋₁₀alkyl” and “C₁₋₆alkyl” refers to a specific alkyl chain length as described herein.

“Alkenyl” whether used alone, or in compound words such as alkenyloxy or haloalkenyl, represents straight or branched chain hydrocarbons containing at least one carbon-carbon double bond, including, unless explicitly limited to smaller groups, moieties ranging in size from two to about 6 carbon atoms or greater, such as, ethylene, 1-propenyl, 2-propenyl, and/or butenyl, pentenyl, hexenyl, and higher isomers, including, e.g., those straight or branched chain hydrocarbons ranging in size, for example, from about 6 to about 10 carbon atoms, or greater.

“Alkynyl” whether used alone, or in compound words such as alkynyloxy, represents straight or branched chain hydrocarbons containing at least one carbon-carbon triple bond, including, unless explicitly limited to smaller groups, moieties ranging in size from, e.g., two to about 6 carbon atoms or greater, such as, ethynyl, 1-propynyl, 2-propynyl, and/or butynyl, pentynyl, hexynyl, and higher isomers, including, e.g., those straight or branched chain hydrocarbons ranging in size from, e.g., about 6 to about 10 carbon atoms, or greater.

“Cycloalkyl” represents a mono- or polycarbocyclic ring system of varying sizes, e.g., from about 3 to about 10 carbon atoms, e.g., cyclopropyl, cyclobutyl, cyclopentyl, cyclohexyl or cycloheptyl. The term cycloalkyloxy represents the same groups linked through an oxygen atom such as cyclopentyloxy and cyclohexyloxy. The term cycloalkylthio represents the same groups linked through a sulfur atom such as cyclopentylthio and cyclohexylthio.

“Aryl” whether used alone, or in compound words such as arylalkyl, aryloxy or arylthio, represents: (i) an optionally substituted mono- or polycyclic aromatic carbocyclic moiety, e.g., of about 6 to about 60 carbon atoms, such as phenyl, naphthyl or fluorenyl; or, (ii) an optionally substituted partially saturated polycyclic carbocyclic aromatic ring system in which an aryl and a cycloalkyl or cycloalkenyl group are fused together to form a cyclic structure such as a tetrahydronaphthyl, indenyl, indanyl or fluorene ring.

“Heterocyclyl” or “heterocyclic” whether used alone, or in compound words such as heterocyclyloxy represents: (i) an optionally substituted cycloalkyl or cycloalkenyl group, e.g., of about 3 to about 60 ring members, which may contain one or more heteroatoms such as nitrogen, oxygen, or sulfur (examples include pyrrolidinyl, morpholino, thiomorpholino, or fully or partially hydrogenated thienyl, furyl, pyrrolyl, thiazolyl, oxazolyl, oxazinyl, thiazinyl, pyridyl and azepinyl); (ii) an optionally substituted partially saturated polycyclic ring system in which an aryl (or heteroaryl) ring and a heterocyclic group are fused together to form a cyclic structure (examples include chromanyl, dihydrobenzofuryl and indolinyl); or (iii) an optionally substituted fully or partially saturated polycyclic fused ring system that has one or more bridges (examples include quinuclidinyl and dihydro-1,4-epoxynaphthyl).

“Heteroaryl” whether used alone, or in compound words such as heteroaryloxy represents: (i) an optionally substituted mono- or polycyclic aromatic organic moiety, e.g., of about 1 to about 10 ring members in which one or more of the ring members is/are element(s) other than carbon, for example nitrogen, oxygen, sulfur or silicon; the heteroatom(s) interrupting a carbocyclic ring structure and having a sufficient number of delocalized pi electrons to provide aromatic character, provided that the rings do not contain adjacent oxygen and/or sulfur atoms. Typical 6-membered heteroaryl groups are pyrazinyl, pyridazinyl, pyrazolyl, pyridyl and pyrimidinyl. All regioisomers are contemplated, e.g., 2-pyridyl, 3-pyridyl and 4-pyridyl. Typical 5-membered heteroaryl rings are furyl, imidazolyl, oxazolyl, isoxazolyl, isothiazolyl, oxadiazolyl, pyrrolyl, 1,3,4-thiadiazolyl, thiazolyl, thienyl, triazolyl, and silole. All regioisomers are contemplated, e.g., 2-thienyl and 3-thienyl. Bicyclic groups typically are benzo-fused ring systems derived from the heteroaryl groups named above, e.g., benzofuryl, benzimidazolyl, benzthiazolyl, indolyl, indolizinyl, isoquinolyl, quinazolinyl, quinolyl and benzothienyl; or, (ii) an optionally substituted partially saturated polycyclic heteroaryl ring system in which a heteroaryl and a cycloalkyl or cycloalkenyl group are fused together to form a cyclic structure such as a tetrahydroquinolyl or pyrindinyl ring.

“Carboxyl” represents a —CO₂H moiety. “Carboxylate” represents a —CO₂ moiety. The two terms are used interchangeably as understood by the person skilled in the art.

“Cyano” represents a —CN moiety.

“Hydroxyl” represents a —OH moiety.

“Alkoxy” represents an —O-alkyl group in which the alkyl group is as defined supra. Examples include methoxy, ethoxy, n-propoxy, iso-propoxy, and the different butoxy, pentoxy, hexyloxy and higher isomers.

“Amino” or “amine” represents an —NH₂ moiety.

“Alkylamino” represents an —NHR or —NR₂ group in which R is an alkyl group as defined supra. Examples include, without limitation, methylamino, ethylamino, n-propylamino, isopropylamino, and the different butylamino, pentylamino, hexylamino and higher isomers.

“Nitro” represents a —NO₂ moiety.

“Amide” represents a —C(O)NR₁R₂ moiety.

“Sulfonyl” represents an —SO₂R group that is linked to the rest of the molecule through a sulfur atom.

“Sulfonamide” represents an —SO₂NR₁R₂ moiety.

“Alkylsulfonyl” represents an —SO₂-alkyl group in which the alkyl group is as defined supra.

The terms “thiol”, “thio”, “mercapto” or “mercaptan” refer to any organosulphur group containing a sulphurhydryl moiety —SH, which includes a R—SH group where R is a moiety containing a carbon atom for covalently bonding to the —SH moiety, for example an alkylsulphur group as defined supra. In one embodiment, the thiol or mercapto group is a sulphurhydryl moiety —SH.

“Alkylthio” represents an —S-alkyl group in which the alkyl group is as defined supra. Examples include, without limitation, methylthio, ethylthio, n-propylthio, iso propylthio, and the different butylthio, pentylthio, hexylthio and higher isomers.

“Cyano” or “nitrile” represents a —CN moiety.

The term “halo” or “halogen” whether employed alone or in compound words such as haloalkyl, haloalkenyl, haloalkynyl, haloalkoxy or haloalkylsulfonyl, represents fluorine, chlorine, bromine or iodine. Further, when used in compound words such as haloalkyl, haloalkenyl, haloalkynyl, haloalkoxy or haloalkylsulfonyl, the alkyl may be partially halogenated or fully substituted with halogen atoms which may be independently the same or different. Examples of haloalkyl include, without limitation, —CH₂CH₂F, —CF₂CF₃ and —CH₂CHFCl. Examples of haloalkoxy include, without limitation, —OCHF₂, —OCF₃, —OCH₂CCl₃, —OCH₂CF₃ and —OCH₂CH₂CF₃. Examples of haloalkylsulfonyl include, without limitation, —SO₂CF₃, —SO₂CCl₃, —SO₂CH₂CF₃ and —SO₂CF₂CF₃.

### Metal Coordination Polymers

The present disclosure provides a metal coordination polymer. A metal coordination polymer is an organometallic polymer structure containing metal atom centres that are linked by linkers/ligands. A metal coordination polymer comprises repeating coordination entities, which can extend in one, two or three directions.

The metal coordination polymers may be at least partially amorphous or at least partially crystalline, for example layered metal coordination polymer having regions of order providing a degree of crystallinity and regions of disorder providing amorphous properties. The metal coordination polymer may be crystalline or amorphous. In one embodiment, the metal coordination polymers are crystalline, for example polycrystalline, and may for example comprise an appropriate amount of homogeneity. In another embodiment, the metal coordination polymers are amorphous. It will be appreciated that crystalline (e.g., polycrystalline) metal coordination polymers are void-containing frameworks comprising an array of metal atoms connected by organic linkers. Amorphous metal coordination polymers still retain the basic building blocks and connectivity of their crystalline counterparts, though they lack any long-range periodic order.

The metal coordination polymer may have a 1D, 2D, or 3D architecture. The metal coordination polymer may comprise two or more 2D metal coordination polymer layers (e.g., a layered metal coordination polymer) or may be a metal organic framework (MOF). In some embodiments the metal coordination polymer is in the form of a 2D sheet. Two or more 2D sheets may electrostatically interact to form a layered metal coordination polymer. The architecture of the metal coordination polymer is generally determined by the metal(s) and ligand(s) used to form the metal coordination polymer.

It will be appreciated that 1D architectures include, for example, a linear structure of metal atoms linked by organic linkers. It will be appreciated that 2D architectures include, for example, a sheet or layer structure having length and width (e.g., area) dimensions of metal atoms linked by organic linkers. The 2D architectures may electrostatically interact to form a layered metal coordination polymer. It will also be appreciated that 3D architectures may form structures, which include, for example, a sphere or cube structure having length, width, and height (e.g., volume) dimensions of metal atoms linked by organic ligands.

In some embodiments, the metal coordination polymer may comprise two or more layers, wherein each layer extends in two dimensions (i.e., a 2D metal coordination polymer layer). Each metal coordination polymer layer may interact (e.g., via electrostatic interactions) to form a layered metal coordination polymer.

In one embodiment, the layered metal coordination polymer may comprise at least 2 layers (e.g., at least two metal coordination polymer layers). The layered metal coordination polymer may be called a bulk layered or stratified metal coordination polymer. The term stratified means formed or arranged into strata or layers. The layered metal coordination polymer may comprise at least 2, 3, 4, 5, 10, 12, 15, 20, 25, 50, 75, 100, 125, 150, 200, 300, 400, or 500 layers. The layered metal coordination polymers may have a range of layers provided by any two of these upper and/or lower layer numbers, for example between about 2 to 500, or about 10 to 200 or 20 to 100 layers. The number of layers may be measured using scanning electron microscopy.

When the metal coordination polymer forms a sheet, a plurality of sheets may assemble to form the layered metal coordination polymer. When a layered structure is formed, some of the organic linkers may be sandwiched between adjacent sheets, and in some embodiments, form an electrostatic interaction (e.g. via one or more labile ions interspersed between the layers).

It will be appreciated that the layered metal coordination polymer may form a structure having any morphology comprising the metal coordination polymer layers that is capable of being exfoliated into individual metal coordination polymer layers. The layered metal coordination polymer can also be disassembled and reassembled in organic solvents, which allow for the morphology of the metal coordination polymer to be modified depending on the conditions.

The layered metal coordination polymer does not have to be planar. In some embodiments the layered metal coordination polymer may be in the form of a tube or rod. For example, the layers may wrap around a central axis of the layered metal coordination polymer. Suitable morphologies may include, but are not limited to sheet-like, hollow, cubic, rod-like, polyhedral, spherical or semi-spherical, rounded or semi-rounded, angular, and irregular morphology, and so forth. For example, the layered metal coordination polymers may form a hexagonal nanotube comprising the layers (e.g., hexagonal Ce-CP nanotube) or irregular layered structures. The layered metal coordination polymer forms a structure that has an aspect ratio (i.e., the ratio of a length to a width, where the length and width are measured perpendicular to one another, and the length refers to the longest linearly measured dimension) of 1.0 to 100.0, 1.0 to 50.0, or 1.0 to 20.0. The morphology may be determined using scanning or transmission electron microscopy.

The layered metal coordination polymer may have an average pore size. In some embodiments, the average pore size of the layered metal coordination polymer may be at least 1, 2, 3, 4, 5, 6, 7, 8, 9, 10, 15, 20, 50, 80, or 100 nm. The average pore size of the layered metal coordination polymer may be less than 100, 80, 50, 20, 15, 10, 9, 8, 7, 6 5, 4, 3, 2, or 1 nm. The pore size may be in a range provided by any two of these upper and/or lower average pore sizes, for example, between about 1 nm to about 50 nm or about 5 nm to about 20 nm. The pore size may be about 1, 2, 3, 4, 5, 6, 7, 8, 9, or 10 nm.

The layered metal coordination polymer may have an average pore volume. The pore volume may be at least about 0.01, 0.1, 0.2, 0.5, 0.8, 1.0, 1.2, 1.5, 2, 5, or 10 cm³/g. The average pore volume may be less than about 10, 5, 2, 1.5, 1.2, 1.0, 0.8, 0.5, 0.2, 0.1, or 0.01 cm³/g. The average pore volume may be in a range provided by any two of these upper and/or lower average pore volumes, for example between about 0.1 to about 2 cm³/g.

The layered metal coordination polymer may have a specific surface area, e.g. a Brunauer-Emmett-Teller (BET) surface area. The specific surface area may be at least about 25, 50, 75, 85, 95, 100, 200, 500, or 1000 m²/g. The specific surface area may be less than about 1000, 500, 200, 100, 95, 85, 75, 50, or 25 m²/g. The specific surface area may be at least about 70, 75, 80, 85, 90, 95, or 100 m²/g. The specific surface area may be in a range provided by any two of these upper and/or lower specific surface areas, for example between about 75 to about 1000 m²/g.

It will be appreciated that the average pore size, pore volume, and specific surface area can be modified depending on the metal atom or organic linker, reagents, solvents and reaction conditions used to prepare the metal coordination polymer layers. The average pore size, pore volume, and specific surface area may be measured by any suitable technique for example gas sorption or scattering techniques.

The layered metal coordination polymer comprises metal atoms coordinated to an organic linker to form a metal coordination polymer layer. The organic linkers of the metal coordination polymers comprise a metal binding moiety and one or more moieties capable of forming an electrostatic interaction with an adjacent metal coordination polymer layer to form the layered metal coordination polymer.

The organic linkers are typically selected from compounds comprising a metal binding moiety. In one embodiment, the organic linker may be selected from a compound comprising one or more carboxylic acid (—COOH)/carboxylate (—COO—), hydroxyl (—OH), amine (—NH₂), nitro (—NO₂), thiol (—SH), or nitrile (—CN) groups. In addition to the metal binding moiety, the organic linker may comprise one or more one or more moieties capable of forming an electrostatic interaction with an adjacent metal coordination polymer layer to form the layered metal coordination polymer.

In one embodiment, the organic linker may be an optionally interrupted alkyl, alkenyl or alkynyl substituted with a metal binding moiety and one or more moieties capable of forming an electrostatic interaction with an adjacent metal coordination polymer layer to form the layered metal coordination polymer. In another embodiment, the organic linker is an optionally interrupted alkyl substituted with a metal binding moiety and one or more moieties capable of forming an electrostatic interaction with an adjacent metal coordination polymer layer to form the layered metal coordination polymer.

In one embodiment, the metal coordination polymer comprises metal atoms coordinated to an organic linker to form a metal coordination polymer layer, wherein the metal atoms comprise one or more metals selected from transition metals, post-transition metals, metalloids, or rare earth metals (including actinides and lanthanides); and the organic linker is selected from an optionally interrupted alkyl, alkenyl or alkynyl substituted with a metal binding moiety and one or more moieties capable of forming an electrostatic interaction with an adjacent metal coordination polymer layer to form the layered metal coordination polymer. The metal atom may be provided by any embodiments or example thereof as described herein.

In one embodiment, the organic linker may be selected from a compound comprising one or more carboxylic acid (—COOH), hydroxyl (—OH), amine (—NH₂), nitro (—NO₂), thiol (—SH), or nitrile (—CN) groups and one or more moieties capable of forming an electrostatic interaction with an adjacent metal coordination polymer layer to form the layered metal coordination polymer. The organic linker may be provided by any embodiments or examples thereof as described herein.

### Metals Used for Metal Coordination Polymers

The layered metal coordination polymer comprises metal atoms coordinated to an organic linker to form a metal coordination polymer layer. The metal atom may be any metal atom suitable to form a coordination network, for example capable of forming a coordinative bond to a metal binding moiety.

In some embodiments, the metal atom may typically comprise one or more metals selected from Group 1 to 16 metals of the Periodic Table and rare earth metals (i.e., actinides and lanthanides).

In some embodiments, the metal atom may typically comprise one or more metals selected from alkali metals, alkali earth metals, transition metals, post-transition metals, metalloids, or rare earth metals (including actinides and lanthanides). Non-limiting metal atoms are those from in the following groups: alkali metals (e.g., Li, Na, K, Rb, Cs, Fr), alkaline earth metals (e.g., Be, Mg, Ca, Sr, Ba, Ra), transition metals (e.g., Sc, Y, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd, Hg), post-transition metals (e.g., Al, Ga, In, Tl, Sn, Pb, Bi), metalloids (e.g., B, Si, Ge, As, Sb, Te, Po, P), and rare earth metals (e.g., La, Ce, Pr, Nd, Pm, Sm, Eu, Gd, Tb, Dy, Ho, Er, Tm, Yb, Lu, Ac, Th, Pa, U, Np, Pu, Am, Cm, Bk, Cf, Es, Fm, Md, No, Lr), and any combinations thereof.

In some embodiments, the metal atom may comprise one or more of a rare earth metal or a transition metal. In embodiments, the metal atom is selected from one or more Ti, V, Cr, Mn, Fe, Co, Ni, Cu, Nb, Mo, Tc, Ru, Rh, Pd, Ag, Ta, W, Re, Os, Ir, Pt, Au, Hg, Ce, Pr, Nd, Pm, Sm, Eu, Gd, Tb, Dy, Ho, Er, Tm, Yb, Sc, Zn, Y, Zr, Cd, Lu, Hf, La, B, Al, Ga, In, Tl, Si, Ge, Sn, Pb, P, As, or Sb. In some embodiments, the metal atom is selected from one or more of Ce, Cu, Mn, Fe, Ni, Zn, Ti, Zr. In some embodiments the metal atom is selected from one or more of Ce, Ti, Zr, or Zn. In one embodiment, the metal atom is Ce. The metal atom may be a single metal atom or a cluster of metal atoms, for example a cluster of two or more different metal atoms described herein.

In some embodiments, the metal atom is a metal ion. The metal ion may be univalent or monovalent (i.e., a metal ion having only one possible charge). The metal ion may be multivalent (i.e., a metal ion can have more than one possible charge, for example more than one oxidation state). The metal ion may have two or more oxidation states.

The metal may be a multivalent ion, wherein the metal ion may be in an unstable/metastable state when the multivalent ion is in a first oxidation state and in a stable state when the multivalent ion is in a second oxidation state. For example, the metal ion may have a first oxidation state when bound to the organic linker and a second oxidation state when the organic linker is removed.

The metal ion may be an ion of any one of the metal atoms described herein. In some embodiments, the metal ion is selected from one or more of alkali metals, alkali earth metals, transition metals, post-transition metals, metalloids, and rare earth metals (including actinides and lanthanides). Non-limiting metal ions are those selected from the following group: alkali metals (e.g., Li, Na, K, Rb, Cs, Fr), alkaline earth metals (e.g., Be, Mg, Ca, Sr, Ba, Ra), transition metals (e.g., Sc, Y, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd, Hg) post-transition metals (e.g., Al, Ga, In, Tl, Sn, Pb, Bi), metalloids (e.g., B, Si, Ge, As, Sb, Te, Po, P), and rare earth metals (e.g., La, Ce, Pr, Nd, Pm, Sm, Eu, Gd, Tb, Dy, Ho, Er, Tm, Yb, Lu, Ac, Th, Pa, U, Np, Pu, Am, Cm, Bk, Cf, Es, Fm, Md, No, Lr), and any combinations thereof.

In some embodiments, the metal ion may comprise one or more of a rare earth metal or a transition metal. In some embodiments, the metal ion is selected from one or more of Ti, V, Cr, Mn, Fe, Co, Ni, Cu, Nb, Mo, Tc, Ru, Rh, Pd, Ag, Ta, W, Re, Os, Ir, Pt, Au, Hg, Ce, Pr, Nd, Pm, Sm, Eu, Gd, Tb, Dy, Ho, Er, Tm, Yb, Sc, Zn, Y, Zr, Cd, Lu, Hf, La, B, Al, Ga, In, Tl, Si, Ge, Sn, Pb, P, As, or Sb. In some embodiments, the metal ion is selected from one or more of Ce, Cu, Mn, Fe, Ni, Zn, Ti, Zr.

By way of example, the metal ion may be one or more of Ce³⁺, Ce⁴⁺, Ti⁴⁺, Zr⁴⁺, or Zn⁺. In one embodiment, the metal ion is Ce³⁺ and/or Ce⁴⁺. The metal atom (including any ion thereof) may be provided as a salt, for example a hydroxide, nitrate, chloride, acetate, oxalate, formate, peroxide, or sulfate salt.

In addition to being coordinated to one or more organic linkers according to any embodiments or examples thereof as described herein, the metal atom may be coordinated to one or more additional organic ligands. These organic ligands may be an oxygen based ligand. For example, the organic ligands may be hydroxyl or water.

### Organic Linker Used for Metal Coordination Polymers

The layered metal coordination polymer comprises metal atoms coordinated to an organic linker to form a metal coordination polymer layer. The organic linker comprises a metal binding moiety. The organic linker may also comprise one or more moieties capable of forming an electrostatic interaction with an adjacent metal coordination polymer layer to form the layered metal coordination polymer. The organic linker stabilises the metal atom by forming a coordinative bond.

The one or more moieties capable of forming an electrostatic interaction with an adjacent metal coordination polymer may form pendant groups (i.e. terminating) at the opposite end of the organic linker to the metal binding moiety. The metal binding moiety forms coordinative bonding (e.g., stronger covalent coordinate bonds) to one or more metals, which can result in stronger intralayer bonding within the metal coordination polymer layer. The one or more moieties capable of forming an electrostatic interaction with an adjacent metal coordination polymer layer can result in weaker inter-layer electrostatic interaction (e.g., weaker Van de Waals interactions). Such weaker electrostatic interactions between layers allow the layered metal coordination polymer to be exfoliated into individual metal coordination polymer layers, which can act as a platform for preparing thin nanostructures. Such weaker interactions also allow for the metal coordination polymer to be disassembled and reassembled into various morphologies.

By way of example only, the organic linker may be trichloroacetic acid, wherein the carboxylic acid group is the metal binding moiety and the trichloromethyl group is the moiety capable of forming an electrostatic interaction with an adjacent metal coordination polymer chain. Alternatively, in another example, the organic linker may be formic acid, wherein the carboxylic acid group is the metal binding moiety and the terminating hydrogen can form an electrostatic interaction with an adjacent metal coordination polymer layer, for example the terminating hydrogen of the organic linker on adjacent metal coordination polymer chains may form an electrostatic interaction with a labile ion interspersed between the layers to hold the layers together. For example, the terminating hydrogen can form an electrostatic interaction with an intra-layer hydroxide ion or oxygen in water, or any other suitable ion capable of forming hydrogen bonding with the terminal hydrogens.

In one embodiment, the metal binding moiety is a different to the one or more moieties capable of forming an electrostatic interaction with an adjacent metal coordination polymer layer.

In some embodiments, the organic linker may be selected from one or more compounds having the structure of Formula 1:

X—R¹ (1)

wherein:

X is a metal binding moiety for coordinative bonding to a metal atom; and

R¹ is H or an optionally interrupted alkyl, alkenyl or alkynyl group substituted with one or more moieties for forming an electrostatic interaction with an adjacent metal coordination polymer layer to form the layered metal coordination polymer.

In some embodiments, R¹ is H or an optionally interrupted alkyl group substituted with one or more moieties for forming an electrostatic interaction with an adjacent metal coordination polymer layer to form the layered metal coordination polymer.

### Metal Binding Moiety (X)

The metal binding moiety (X) may be any suitable moiety for forming a coordinative bond to one or more metal atoms. In some embodiments, the coordinative bonding of the metal binding moiety to the metal atom may be a direct bond, e.g., by covalent coordinate bond or metal ligand bond, or an indirect bond, e.g., by weaker electrostatic interactions (e.g., hydrogen bond, halogen bond), van der Waals interactions (e.g., dipole-dipole, dipole-induced dipole, London dispersion). In one embodiment, the metal binding moiety forms covalent coordinate bonds with the one or more metal atoms. The metal binding moiety may be a head group on the organic linker, wherein the organic linker comprises a tail. The tail may be H or an optionally interrupted and substituted alkyl, alkenyl or alkynyl according to any embodiments or examples thereof as described herein.

The metal binding moiety may be a monodentate, bidentate, or polydentate ligand. In some embodiments, the metal binding moiety is a monodentate or a bidentate ligand. The monodentate or bidentate ligand may form a bridging coordinative bond to two or more metal atoms to form the metal coordination polymer layer.

The metal binding moiety may have one site that can coordinate with one or more metal atoms. When these organic linkers are used and the metal coordination polymer is in the form of a plurality of sheets, each sheet may be at least partially covered by organic linkers. In some embodiments the organic linkers have two or more sites that can coordinate with one or more metal atoms (e.g., carboxylic acid/carboxylate moieties).

In some embodiments, the metal binding moiety comprises a metal donor atom. In some embodiments, the metal donor atom is a heteroatom. In some embodiments, the metal donor atom is selected from the group consisting of oxygen, nitrogen, sulfur, selenium, silicon, or tellurium. In some embodiments, the metal donor atom is sulfur, nitrogen or oxygen. In one embodiment, the metal donor atom is oxygen. In some embodiments, the metal donor atom is a heteroatom in a heteroalkyl, heterocyclyl, or heteroaryl.

In some embodiments, the metal binding moiety comprises carboxylic acid (—COOH), hydroxyl (—OH), amine (—NH₂), nitro (—NO₂), thiol (—SH), nitrile (—CN), substituted or unsubstituted heterocyclyl, or substituted or unsubstituted heteroaryl. In some embodiments, the metal binding moiety is carboxylic acid (—COOH). It will be appreciated that that various metal binding moieties, which include hydrogen (e.g., carboxylic acid —COOH), may also be written without the hydrogen (e.g., carboxylate —COO⁻). For example, carboxylic acid may form a monodentate coordinate bond to one or more metal atoms wherein the hydrogen is retained. Alternatively, carboxylic acid may form a monodentate or bidentate coordinative bond to one or more metal atoms via a carboxylate anion. Reference to carboxylate herein also refers to carboxylic acid, and the two may be used interchangeably, as understood by the person skilled in the art.

In one embodiment, the metal binding moiety may be bidentate. Any suitable bidentate metal binding moiety can be used, for example, the bidentate metal binding moiety may comprise a carboxylic acid (—COOH)/carboxylate (—COO—), amine (—NH₂) (including for example a primary amine (—NH₂), secondary amine (—NH), tertiary amine (—N(R)—)), thiol (—SH), hydroxyl (—OH), or nitrile (—CN).

In one embodiment, the metal binding moiety comprises a carboxylic acid group (which may be deprotonated under certain bonding conditions to form a carboxylate group). The carboxylic acid/carboxylate metal binding moiety of each organic linker may independently form a monodentate or a bidentate coordination bond to one or more metal atoms. In some embodiments, the carboxylic acid/carboxylate metal binding moiety forms a bridging coordinative bond to at least two metal atoms to form the metal coordination polymer layer.

In some embodiments, the metal binding moiety may comprise a carboxylic acid, carboxylate, acetate, oxalate, acetylacetonate, or chatecholate. In one embodiment, the metal binding moiety is a carboxylic acid and/or carboxylate.

### R¹ Group

The organic linker may comprise one or more moieties for forming an electrostatic interaction with an adjacent metal coordination polymer layer to form the layered metal coordination polymer. The one or more moieties may be attached directly to the metal binding moiety. Alternatively, the one or more moieties may be attached to the metal binding moiety via the R¹ group as defined above or herein. When present and not H, the R¹ group can be substituted by the one or more moieties.

In some embodiments, the organic linker comprises an R¹ group attached to the metal binding moiety. R¹ can be any type of unsaturated or saturated organic molecule. In some embodiments, R¹ is H. In some embodiment, R¹ is an optionally interrupted alkyl, alkenyl or alkynyl group substituted with one or more moieties for forming an electrostatic interaction with an adjacent metal coordination polymer layer to form the layered metal coordination polymer. It will be appreciated that where R¹ is hydrogen, the hydrogen may still form an electrostatic interaction with the adjacent metal coordination polymer to form the layered metal coordination polymer. In another embodiment, R¹ is an optionally interrupted alkyl group substituted with one or more moieties for forming an electrostatic interaction with an adjacent metal coordination polymer layer to form the layered metal coordination polymer.

The optionally interrupted alkyl, alkenyl or alkynyl group may be selected from an optionally interrupted C₁₋₂₀alkyl, C₂₋₂₀alkenyl or C₂₋₂₀alkynyl group each substituted with one or more moieties for forming an electrostatic interaction with an adjacent metal coordination polymer layer to form the layered metal coordination polymer. The optionally interrupted alkyl, alkenyl or alkynyl group may be selected from an optionally interrupted C₁₋₁₀alkyl, C₂₋₁₀alkenyl or C₂₋₁₀alkynyl group each substituted with one or more moieties for forming an electrostatic interaction with an adjacent metal coordination polymer layer to form the layered metal coordination polymer. The optionally interrupted alkyl, alkenyl or alkynyl group may be selected from an optionally interrupted C₁₋₆alkyl, C₂₋₆alkenyl or C₂₋₆alkynyl group each substituted with one or more moieties for forming an electrostatic interaction with an adjacent metal coordination polymer layer to form the layered metal coordination polymer. The substituted one or more moieties for forming an electrostatic interaction with an adjacent metal coordination polymer layer to form the layered metal coordination polymer can be provided according to any embodiments or examples thereof as described herein.

In an embodiment, R¹ is H or an optionally interrupted C₁₋₂₀alkyl substituted with one or more moieties for forming an electrostatic interaction with an adjacent metal coordination polymer layer to form the layered metal coordination polymer.

In an embodiment, R¹ is H or an optionally interrupted C₁₋₁₀alkyl substituted with one or more moieties for forming an electrostatic interaction with an adjacent metal coordination polymer layer to form the layered metal coordination polymer.

In an embodiment, R¹ is H or an optionally interrupted C₁₋₆alkyl, substituted with one or more moieties for forming an electrostatic interaction with an adjacent metal coordination polymer layer to form the layered metal coordination polymer.

In an embodiment, R¹ is H, or an optionally interrupted methyl, ethyl, propyl, butyl, pentyl, or hexyl substituted with one or more moieties for forming an electrostatic interaction with an adjacent metal coordination polymer layer to form the layered metal coordination polymer.

In an embodiment, R¹ is H or methyl substituted with one or more moieties for forming an electrostatic interaction with an adjacent metal coordination polymer layer to form the layered metal coordination polymer.

In some embodiments, the alkyl group of each R¹ of the organic linker as described above may be optionally interrupted with one or more heteroatom, including one or more O, N, S, Se, Te, Si, and/or one or more alkenyl, alkynyl, aryl, heteroaryl, heteroaryl, and/or cycloalkyl, in which each alkenyl, alkynyl, aryl, heteroaryl, or cycloalkyl group may be optionally substituted.

In some embodiments, the one or more moieties substituted on R¹ may be any suitable ion capable of forming an electrostatic interaction an adjacent metal coordination polymer chain, for example with an oppositely charged ion either on the adjacent metal coordination polymer chain and/or via one or more labile protons that are interspersed between the metal coordination polymer layers.

In some embodiments, the one or more moieties substituted on R¹ form a hydrogen bond, halogen bond, van der Waals interactions (e.g., dipole-dipole, dipole-induced dipole, London dispersion) with an adjacent metal coordination polymer layer.

In one embodiment, the one or more moieties substituted on R¹ form van der Waals interactions with an adjacent metal coordination polymer layer. Such interactions may form via one or more labile protons that are interspersed between the metal coordination polymer layers.

In some embodiments, R¹ is terminated with one or more moieties for forming an electrostatic interaction with an adjacent metal coordination polymer layer. For example, the terminating hydrogen ions of the alkyl of R¹ can be substituted with one or more moieties for forming an electrostatic interaction with an adjacent metal coordination polymer layer. The terminating hydrogen ions of R¹ may be substituted with a more electronegative moiety, for example a halogen-based moiety, including one or more of halogen, haloalkyl, haloalkenyl, haloalkynyl, haloalkoxy, haloalkylsulfonyl, or other suitable moieties described herein. Alternatively, the terminating hydrogen ions of R¹ may be substituted with a less electronegative moiety, for example one or more halides selected from the group consisting of Li, Na, K, Rb, or Cs.

In some embodiments, the one or more moieties substituted on R¹ form an electrostatic interaction with labile ions interspersed between the metal coordination polymer layers to from the layered metal coordination polymer.

In some embodiments, the one or more moieties substituted on R¹ for forming an electrostatic interaction with an adjacent metal coordination polymer may be selected from the group consisting of halogen, haloalkyl, haloalkenyl, haloalkynyl, haloalkoxy, haloalkylsulfonyl, nitrile, hydroxyl, amine, carboxyl, carboxylate, amide, nitro, thiol, sulphonamide, or sulfonyl. In some embodiments, the one or more moieties substituted on R¹ for forming an electrostatic interaction with an adjacent metal coordination polymer may be selected from the group consisting of halogen, haloalkyl, haloalkenyl, haloalkynyl, haloalkoxy, or haloalkylsulfonyl.

In some embodiments, the one or more moieties substituted on R¹ for forming an electrostatic interaction with an adjacent metal coordination polymer may be selected from the group consisting of halogen, C₁₋₂₀haloalkyl, C₂₋₂₀haloalkenyl, C₂₋₂₀haloalkynyl, C₁₋₂₀haloalkoxy, or C₁₋₂₀haloalkylsulfonyl. In some embodiments, the one or more moieties substituted on R¹ for forming an electrostatic interaction with an adjacent metal coordination polymer may be selected from the group consisting of halogen, C₁₋₁₀haloalkyl, C₂₋₁₀haloalkenyl, C₂₋₁₀haloalkynyl, C₁₋₁₀haloalkoxy, or C₁₋₁₀haloalkylsulfonyl. In some embodiments, the one or more moieties substituted on R¹ for forming an electrostatic interaction with an adjacent metal coordination polymer may be selected from the group consisting of halogen, C₁₋₆haloalkyl, C₂₋₆haloalkenyl, C₂₋₆haloalkynyl, C₁₋₆haloalkoxy, or C₁₋₆haloalkylsulfonyl.

In some embodiments, the one or more moieties substituted on R¹ for forming an electrostatic interaction with an adjacent metal coordination polymer may be selected from the group consisting of halogens. In some embodiments, the one or more moieties substituted on R¹ for forming an electrostatic interaction with an adjacent metal coordination polymer may be selected from —F, —Cl, —Br, or —I.

In some embodiments, the one or more moieties substituted on R¹ for forming an electrostatic interaction with an adjacent metal coordination polymer may be selected from the group consisting of —F, —Cl, —Br, —I, —CF₃, —Cl₃, —CCl₃, —CBr₃, —CHF₂, —CHCl₂, —CHI₂, —CHBr₂, —OCH₂F, —OCH₂Cl, —OCH₂I, —OCH₂Br, —OCHF₂, —OCHCl₂, —OCHI₂, —OCHBr₂, —OCF₃, —OCl₃, —OCl₃, —OCBr₃, —CN, —OH, —NH₂, —COOH, —CONH₂, —NO₂, —SH. —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocyclyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In some embodiments, the one or more moieties substituted on R¹ for forming an electrostatic interaction with an adjacent metal coordination polymer may be selected from is selected from the group consisting of —F, —Cl, —Br, —I, —CF₃, —Cl₃, —CCl₃, —CBr₃, —CHF₂, —CHCl₂, —CHI₂, —CHBr₂, —OCH₂F, —OCH₂Cl, —OCH₂I, —OCH₂Br, —OCHF₂, —OCHCl₂, —OCHI₂, —OCHBr₂, —OCF₃, —OCl₃, —OCl₃, and —OCBr₃.

In some embodiments, R¹ is selected from the group consisting of H, alkyl, alkenyl, alkynyl, alkoxy, haloalkyl, haloalkenyl, haloalkynyl, haloalkoxy, haloalkylsulfonyl, alkylamine, alkylcarboxylic acid, alkylamide, alkylthiol, alkylsulphonamide, or alkylsulfonyl.

In some embodiments, R¹ is selected from the group consisting of H, alkyl, haloalkyl, haloalkoxy, alkylamine, alkylcarboxylic acid, alkylthiol, alkylsulphonamide, or alkylsulfonyl. In some embodiments, R¹ is selected from the group consisting of H, alkyl, halogen, haloalkyl, and alkylcarboxylic acid.

In some embodiments, R¹ is selected from the group consisting of H, C₁₋₂₀alkyl, C₁₋₂₀haloalkyl, and C₁₋₂₀carboxylic acid. In some embodiments, R¹ is selected from the group consisting of H, halogen, C₁₋₁₀alkyl, C₁₋₁₀haloalkyl, and C₁₋₁₀carboxylic acid.

In some embodiments, R¹ is selected from the group consisting of —CF₃, —Cl₃, —CCl₃, —CBr₃, —CHF₂, —CHCl₂, —CHI₂, —CHBr₂, —OCH₂F, —OCH₂Cl, —OCH₂I, —OCH₂Br, —OCHF₂, —OCHCl₂, —OCHI₂, —OCHBr₂, —OCF₃, —OCl₃, —OCl₃, —OCBr₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In some embodiments, the metal atom is an ion selected from Ce³⁺, Ce⁴⁺, Ti⁴⁺, Zr⁺ or Zn⁺, X is —COOH, —SH, —NH₂, —OH, and R¹ is selected from —CF₃, —Cl₃, —CCl₃, —CBr₃, —CHF₂, —CHCl₂, —CHI₂, —CHBr₂, —OCH₂F, —OCH₂Cl, —OCH₂I, —OCH₂Br, —OCHF₂, —OCHCl₂, —OCHI₂, —OCHBr₂, —OCF₃, —OCl₃, —OCl₃, or —OCBr₃.

In some embodiments, the organic linkers are organic-based. In some embodiments, the organic linkers include an alkyl-, alkene-, alkyne- and/or aryl-based carboxylic acid. For example, the organic linkers may be a halide-substituted alkyl acid, such as trichloroacetic acid. In some embodiments the organic linkers include formic acid. In some embodiments the organic linkers may act as a Lewis acid and the metal atom acts as a Lewis base, or vice versa.

In some embodiments, the organic linker is a carboxylic acid. In some embodiments the organic linker is formic acid, trifluoroacetic acid, trichloroacetic acid, tribromoacetic acid, or triiodoacetic acid. In one embodiment, the organic linker is formic acid or trichloroacetic acid. In one embodiment, the organic linker is trichloroacetic acid.

To obtain a layered metal coordination polymer wherein each layer comprises stronger intra-layer coordinative covalent bonding between the organic linker and one or more metal atoms throughout the layer and weaker electrostatic interactions between layers, in some embodiments the organic linker does not form a coordinative bond to a metal atom of an adjacent metal coordination polymer layer. This allows for each layer of the layered metal coordination polymer to be held together by weak Van de Waals interactions between each layer.

### Layered Metal Coordination Polymer

The layered metal coordination polymer may be an unstable layered metal coordination polymer. An unstable layered metal coordination polymer comprises a metal centre or substructure (e.g., [Ce(OH)₂]²⁺) that is inherently unstable but can exist indefinitely owing to the presence of the organic linkers, which effectively “cap” and stabilise the unstable metal centre or substructure of the coordination polymer. The unstable metal centre or substructure may also be called a “reactive metal-based species”. Upon removal of the stabilising or “capping” organic linker, the metal has a tendency to form a more stable metal-based species, such as a nanostructure. For example, the reactive metal-based species may be a metal atom (e.g., a metal ion) having unsaturated coordination number that has a tendency to make covalent bonds to fill up the coordination sites when the organic linker is removed. Conversion from the unstable/metastable to a stable state may be achieved through an intermediate. For example, in some embodiments, the reactive metal-based species may have a tendency to form an unstable/metastable intermediate that quickly converts to the more stable metal-based species upon removal of the organic linker. However, an unstable/metastable intermediate is not formed in all embodiments. The terms “unstable” and “metastable” are used interchangeably throughout this disclosure.

Prior to the present disclosure, unstable metal coordination polymers were previously avoided as potential precursors to form nanostructures as their properties meant that they could not be used as nanostructured materials for any duration of time. However, an advantage of this instability is that unstable metal coordination polymers can be used as a precursor materials to make other nanostructures with specific structures.

Unstable metal coordination polymers may also allow a structure or architecture of the metal coordination polymer to be retained during the formation of the nanostructure. This retention of structure is something that was not previously envisaged with the use of unstable metal coordination polymers. The use of unstable metal coordination polymers as a precursor may also allow for the formation of polycrystalline nanostructures, which is something that was not previously considered for unstable metal coordination polymers.

The reactive metal-based species comprising the metal coordinated to the organic linker forms part of the metal coordination polymer. The reactive metal-based species is stabilised by the organic linker so that the reactive metal-based species can exist in the “reactive” (or metastable) state. Removal of the organic linker allows the unstable metal-based species to adopt a more stable state (i.e., the more stable metal-based species). In some embodiments, the organic linker stabilizes the metal atom by forming coordinative bonding of the metal atom to the organic linker. In other words, the ligands help to “cap” the reactive metal-based species to prevent the reactive metal-based species from forming the more stable metal-based species. Removal of such capping linkers results in the conversion of the unstable metal-based species to a more stable metal nanostructure.

In some embodiments, the layered metal coordination polymer comprises a plurality of labile ions interspersed between the metal coordination polymer layers. The term labile ions refers to ions that can disperse throughout the interlayer space between the layers of the metal coordination polymer. The labile ions can form the electrostatic interaction between the one or more moieties of the organic linker of each metal coordination polymer layer to form the layered metal coordination polymer. The labile ions may be have a positive charge or a negative charge. The labile ions may be acidic protons (H⁺). The protons may be introduced in-situ during the synthesis of the metal coordination polymers. The labile ions may form an electrostatic interaction with one or more moieties that are terminating the organic linker to form the layered metal coordination polymer. For example, when the organic linker is acidic (e.g., trichloroacetic acid), the labile ions may be protons of the acid and may be intercalated between the sheets. The intercalated protons may help to keep the layered material together. For example, the protons may act as weak electrostatic crosslinking agents.

The labile ions may be of opposite charge to the terminating one or more moieties of the organic linker. For example, where the organic linker is terminated with one or more negatively charged ions, including for example halogen moieties (e.g., F, Cl, Br, I), the labile ions may be positive, for example protons (H⁺). Alternatively, if the organic linker is terminated with hydrogen or one or more positive ions (e.g., Li, Na, K, Rb, and/or Cs), the labile ions may be negatively charged (e.g. OH⁻). The labile ions may originate from the carboxylic acid metal binding moiety of the organic linker and/or the metal source used to prepare the metal coordination polymer, and/or the solvent system used to prepare the metal coordination polymer (e.g. H₂O).

In some embodiments, the electrostatic interaction between the labile ions and the one or more terminating moieties of the organic linker may be substantially orthogonal (e.g. perpendicular) to the coordinative bonding within the metal coordination polymer. Such orientation of the inter-layer and intra-layer bonding results in the ability to exfoliate the layered metal coordination polymer to one or more individual metal coordination polymer layers under relatively facile conditions.

Owing to the presence of the labile ions (for example protons) between the layers, the metal coordination polymer has a surface charge. In some embodiments the surface charge is positive The surface charge may be positive or negative. The surface charge may be positive. The layered metal coordination polymer may have a zeta potential (which is indicative of surface charge). The layered metal coordination polymer may have a zeta potential of greater than zero (0) mV. In some embodiments, the layered metal coordination polymer has a zeta potential of at least 1, 2, 5, 10, 15, 20, 30, 40, 50, 60, 80, or 100 mV. In some embodiments, the layered metal coordination polymer has a zeta potential of less than 100, 80, 60, 50, 40, 30, 20, 15, 10, 5, 2, or 1 mV. Combinations of any two or more of these upper and/or lower zeta potential values are also possible, for example between about 5 mV to about 100 mV, 5 mV to about 80 mV, or about 10 mV to about 60 mV, e.g., about +30 mV. FIG. 69 shows the zeta potential of a layered metal coordination polymer according to at least some embodiments or examples described herein.

In some embodiments, the metal coordination polymer may be a metal coordination polymer layer that is not electrostatically linked to another layer (i.e. is not cross-linked to form a bulk layered polymer). For example, a layered metal coordination polymer may be exfoliated to obtain one or more individual metal coordination polymer layers.

In some embodiments, the metal coordination polymer is a non-crosslinked metal coordination polymer comprising metal atoms coordinated to an organic linker to form a metal coordination polymer layer, wherein the organic linker is described herein. The metal coordination polymer layer may be a planar or linear layer. In some embodiments, two or more metal coordination polymer layers may electrostatically interact (i.e. cross-link) to form a layered metal coordination polymer, wherein the metal coordination polymer layers are held together by an electrostatic interaction between the organic linker on each metal coordination polymer layer, as described herein.

It will be appreciated that the metal coordination polymers can incorporate other organic ligands that coordinate to one or more metal atoms in addition to the organic linkers, for example negatively charged ions, negatively charged complexes, and/or molecules with a dipole (e.g., water and/or hydroxide ions), and for example may originate from metal salts and or solvents used to prepare the metal coordination polymers.

In some embodiments, each metal atom of the metal coordination polymer may be independently coordinated to at least 5, 6, 7, or 8 atoms from the metal binding moiety and/or one or more additional organic ligands. In some embodiments, each metal atom of the metal coordination polymer may be independently coordinated to at least 7 or 8 atoms from the metal binding moiety of one or more organic linkers and/or one or more additional organic ligands.

In one embodiment, the metal coordination polymer is a cerium metal coordination polymer having the formula Ce(TCA)₂(OH)₂.2H₂O. The cerium metal coordination polymer may be characterised by an X-ray powder diffraction (XRD) pattern comprising one or more principal peaks located at about 7.2, 8.1, 10.9, 20.6, 22.0, 23.1, and/or 23.2 degrees 2θ. Any one or more of these peaks can be used to characterise the cerium metal coordination polymer. The cerium metal coordination polymer may be characterised by the XRD pattern provided in FIG. 8.

The layered metal coordination polymer comprises layers having a certain thickness across the layer, referred to as an axial thickness across the c-axis of the metal coordination polymer layer. In some embodiments, each metal coordination polymer layer may independently have an axial thickness along the c-axis of less than 100, 70, 50, 20, 15, 10, 8, 6, 4, 2, or 1 nm, for example less than 20, 15, 12, 10, 8, 5, 2, or 1 nm. Combinations of any two of these upper and/or lower thickness can provide a range selection, for example between about 1 nm to about 12 nm. In one embodiment, each metal coordination polymer layer may independently have an axial thickness of about 1.1, 2.2, 5.5 or 11 nm, wherein the thickness is proportional to the thickness of one unit cell of the metal coordination polymer. In some embodiments, each metal coordination polymer layer may be about 1, 2, 3, 4, 5, 6, 7, 8, 9, or 10 unit cells thick. The thickness may be measured using scanning electron microscopy or atomic force microscopy (AFM).

### Method for Preparing Metal Coordination Polymers

One main goal of the method for preparing the metal coordination polymers described herein is to establish synthetic conditions that can generate a layered metal coordination polymer that is held together by weak electrostatic interactions. Depending on the reaction conditions, an unstable metal coordination polymer can be prepared, which can be used to prepare a variety of nanostructures.

The metal coordination polymers described herein may be prepared by combining (i.e., contacting) a metal atom source and an organic linker to form a layered metal coordination polymer comprising two or more metal coordination polymer layers that are held together by an electrostatic interaction. The term “combining” or “contacting” may include allowing two species to react, interact, or physically touch, wherein the two species may be an organic linker and metal atom as described herein, and in some cases one or more other species including a gas, for example oxygen.

The metal atom source may comprise any metal atom (e.g. metal ion) as described herein for the metal coordination polymer, including those described under the heading “Metals used for metal coordination polymer”. The metal atom source may typically comprise one or more metals selected from alkali metals, alkali earth metals, transition metals, post-transition metals, metalloids, or rare earth metals (including actinides and lanthanides). Non-limiting metal atoms are those from in the following groups: alkali metals (e.g., Li, Na, K, Rb, Cs, Fr), alkaline earth metals (e.g., Be, Mg, Ca, Sr, Ba, Ra), transition metals (e.g., Sc, Y, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd, Hg), post-transition metals (e.g., Al, Ga, In, Tl, Sn, Pb, Bi), metalloids (e.g., B, Si, Ge, As, Sb, Te, Po, P), and rare earth metals (e.g., La, Ce, Pr, Nd, Pm, Sm, Eu, Gd, Tb, Dy, Ho, Er, Tm, Yb, Lu, Ac, Th, Pa, U, Np, Pu, Am, Cm, Bk, Cf, Es, Fm, Md, No, Lr), and any combinations thereof. The metal atom source may comprise an ion of any one or more metals described herein. The metal ion may be univalent or monovalent (i.e., a metal ion having only one possible charge). The metal ion may be multivalent (i.e., a metal ion can have more than one possible charge, for example more than one oxidation state). The metal ion may have two or more oxidation states. The metal atom source may comprise a multivalent ion.

In some embodiments, the metal ion source may comprise one or more of a rare earth metal or a transition metal. In some embodiments, the metal ion is selected from one or more of Ti, V, Cr, Mn, Fe, Co, Ni, Cu, Nb, Mo, Tc, Ru, Rh, Pd, Ag, Ta, W, Re, Os, Ir, Pt, Au, Hg, Ce, Pr, Nd, Pm, Sm, Eu, Gd, Tb, Dy, Ho, Er, Tm, Yb, Sc, Zn, Y, Zr, Cd, Lu, Hf, La, B, Al, Ga, In, Tl, Si, Ge, Sn, Pb, P, As, or Sb. In some embodiments, the metal ion is selected from one or more of Ce, Cu, Mn, Fe, Ni, Zn, Ti, Zr. By way of example, the metal ion source may comprise one or more of Ce³⁺, Ce⁴⁺, Ti⁴⁺, Zr⁴⁺, or Zn⁺. In one embodiment, the metal ion is Ce³⁺ and/or Ce⁴⁺. The metal atom source (including any ion thereof) may be provided as a salt of any one or more of the metals described herein, for example a hydroxide, nitrate, chloride, acetate, oxalate, formate, peroxide, or sulfate salt.

The organic linker may be any organic linker as described herein for the metal coordination polymer. The organic linker comprises a metal binding moiety. The organic linker may also comprise one or more moieties capable of forming an electrostatic interaction with an adjacent metal coordination polymer layer to form the layered metal coordination polymer. In some embodiments, the organic linker may be selected from one or more compounds having the structure of Formula 1:

X—R¹ (1)

wherein:

X is a metal binding moiety for coordinative bonding to a metal atom; and

R¹ is H or an optionally interrupted alkyl, alkenyl or alkynyl group substituted with one or more moieties for forming an electrostatic interaction with an adjacent metal coordination polymer layer to form the layered metal coordination polymer. In some embodiments, R¹ is H or an optionally interrupted alkyl group substituted with one or more moieties for forming an electrostatic interaction with an adjacent metal coordination polymer layer to form the layered metal coordination polymer. The metal binding moiety and R¹ may be selected from the binding moieties and R¹ described herein for the metal coordination polymer, including those described under the heading “Organic linker used for metal coordination polymers”. In some embodiments, the organic linker is a carboxylic acid. In some embodiments the organic linker is formic acid, trifluoroacetic acid, trichloroacetic acid, tribromoacetic acid, or triiodoacetic acid. In one embodiment, the organic linker is formic acid or trichloroacetic acid. In one embodiment, the organic linker is trichloroacetic acid.

The combining of the metal atom source and organic linker may include mixing the metal atom source and organic linker. Solvent-free conditions may be used to mix the metal atom source and organic linker, such as sol-gel techniques. Alternatively, an aqueous solution or solvent may be used to mix the metal atom source and organic linker. The mixture may then be heated. Suitable techniques to form the layered metal coordination polymer include hydrothermal, solvothermal, and electrodeposition processes. A polar solvent (e.g., water or organic solvent) may be used to form the mixture of the metal atom source and organic linker. For example, when the organic linker is an organic acid, a metal salt (e.g., metal atom source) and the organic acid may be mixed.

In some embodiments, the process may comprise mixing an aqueous solution comprising a metal atom source and an organic linker to form a layered metal coordination polymer comprising two or more metal coordination polymer layers that are held together by an electrostatic interaction. In some embodiments, the step of forming the layered metal coordination polymer comprises heating the aqueous solution comprising the metal atom source and organic linker.

The reaction conditions may be dependent upon the type of metal coordination polymer that is to be formed. In some embodiments, a mixture of the metal atom source and organic linker may be subjected to hydrothermal or solvothermal treatment.

In one embodiment, the step of forming the layered metal coordination polymer comprises electrodeposition, for example for preparing cerium based metal coordination polymers.

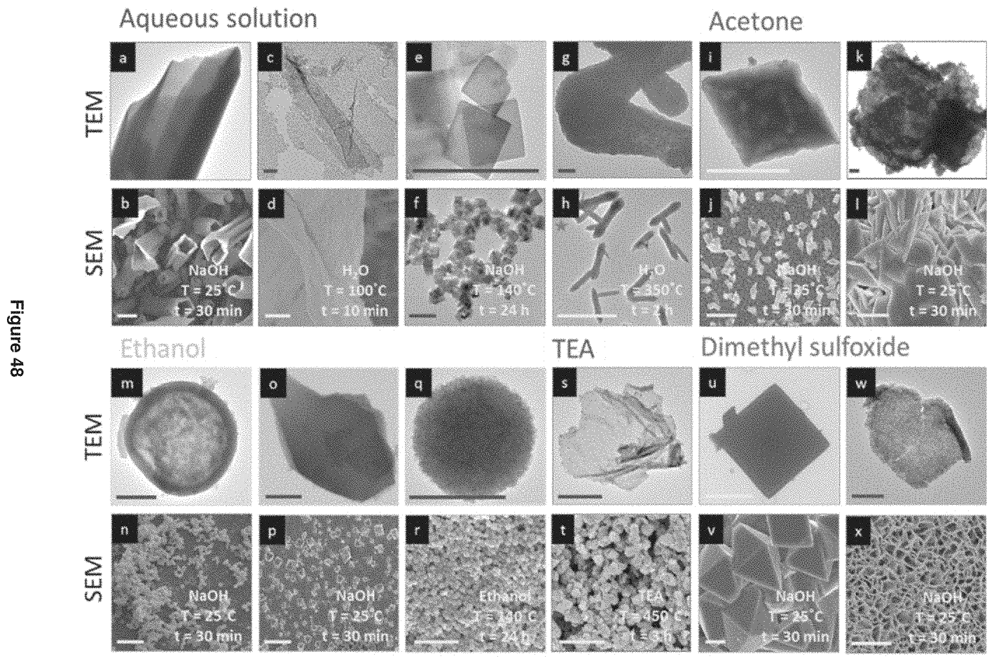

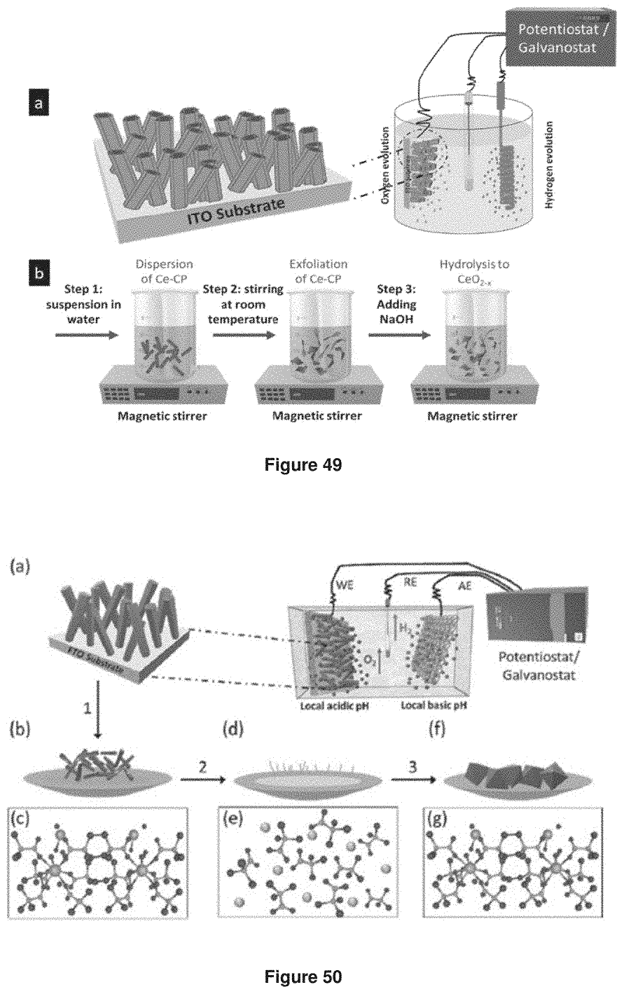

The electrodeposition may be modified anodic chronoamperometric electrodeposition (MACE). The electrodeposition process comprises three-electrodes, and can include a fluorine-doped tin oxide on glass working electrode, platinum wire counter electrode, and Ag/AgCl reference electrode. Other electrodes may also be used. An example of a suitable electrodeposition setup is provided in FIGS. 49 and 50, however this is not to be considered limiting.

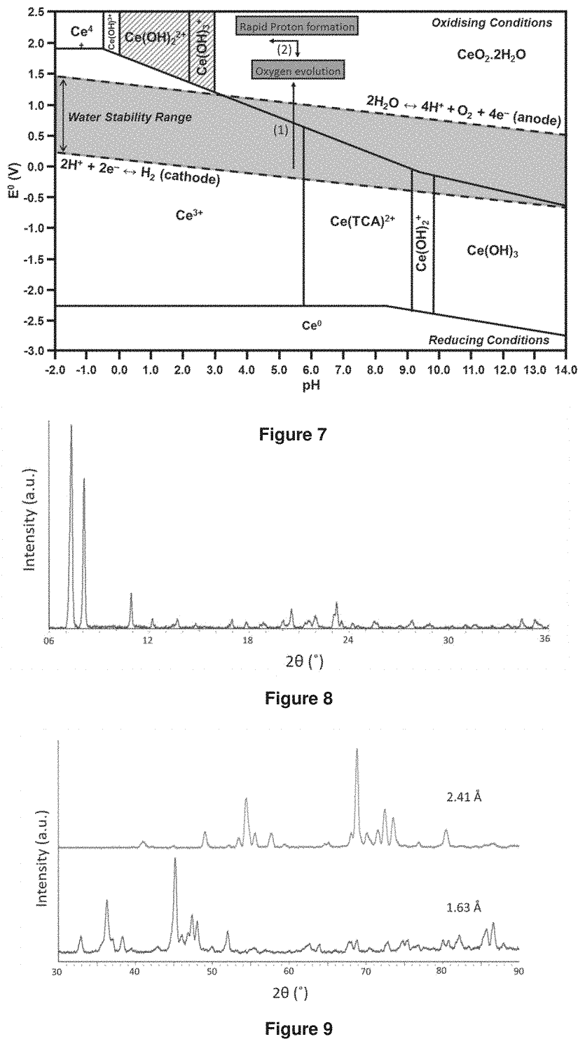

The MACE may be performed within the oxygen evolution region of the aqueous solution comprising the metal atom source and organic linker. The oxygen evolution region will vary depending on the metal atom and organic linker system, which however can readily be determined using Pourbaix diagrams available to the person skilled in the art. An example of a Pourbaix diagram for cerium and trichloroacetic acid is provided in FIG. 7, this however is not to be considered limiting. By performing the electrodeposition in the oxygen evolution region of the aqueous solution comprising the metal atom and organic linker, oxygen molecules are generated at the working electrode which result in the oxidation of the metal species (e.g., Ce(III) to Ce(IV) allowing for the formation of the unstable metal coordination polymer.

The concentration of the metal atom source and organic linker in the aqueous solution are each limited by the maximal solubility of the precursor water-soluble salt that is used as metal atom source. In some embodiments, the concentrations of the metal atom source and organic linker in the aqueous solution are each independently at least about 0.001, 0.01, 0.02, 0.05, 0.08, 0.1, 0.2, 0.4, 0.5, 0.8, or 1 M. In some embodiments, the concentrations of the metal atom source and organic linker in the aqueous solution are each independently less than about 1, 0.8, 0.5, 0.4, 0.2, 0.1, 0.08, 0.05, 0.02, 0.01, or 0.001 M. Combinations of any two or more of these upper and/or lower concentrations are also possible, for example between about 0.001 M to about 1 M or about 0.01 M to about 0.1 M.

The initial pH of the aqueous solution or mixture may be adjusted. In some embodiments, the initial pH of the aqueous solution during electrodeposition may be an acidic pH, for example less than about pH 7. The pH may be adjusted by adding a suitable amount of acid or base depending on the acidity of the aqueous solution comprising the metal atom source and organic linker. In some embodiments, the initial pH of the aqueous solution during electrodeposition may be less than about 7, 6, 5, 4, 3, or 2. Combinations of these pH values are also possible, for example the initial pH of the aqueous solution during electrodeposition may be between about pH 2 to about pH 7, about pH 3 to about pH 7, about pH 5 to about pH 6, for example about pH 4, 4.2, 4.4, 4.6, 4.8, 5, 5.2, 5.4, 5.6, 5.8, 6.0, 6.2, 6.4, 6.8, or 7.0.

The electrodeposition is performed using a constant applied voltage effective to maintain the oxygen evolution region of the aqueous solution comprising the metal atom source and the organic linker. The voltage used for electrodeposition may be determined by the surface area of the working electrode. The voltage may be proportional to the dimensions of the working electrode. The voltage used for electrodeposition may be determined by the aqueous or solvent system used in electrodeposition. In one embodiment, the voltage (i.e., potential) used for electrodeposition may be within an oxygen evolution region of the aqueous solution comprising the metal ion and organic linker, for example as determined by a Pourbaix diagram.

In some embodiments, the electrodeposition is performed using a constant applied voltage of at least about 0.001, 0.01, 0.05, 0.1, 0.2, 0.5, 0.8, 1, 2, 3, 4, 5, 6, 7, 8, 9, or 10 V vs Ag/AgCl. In some embodiments, the electrodeposition is performed using a constant applied voltage of less than 10, 9, 8, 7, 6, 5, 4, 3, 2, 1, 0.8, 0.5, 0.2, 0.1, 0.05, 0.01, or 0.001 V vs Ag/AgCl. Combinations of any two or more of these upper and/or lower voltages are also possible, for example between about 1.0 V to 10.0 V, about 1.0 V to 5.0 V, or about 1.0 V to 2.0 V. Other applied voltages are also possible depending on the metal atom and organic linker, and can be selected based on a suitable Pourbaix diagram for a given metal and organic linker system as appreciated by the skilled person. By applying a voltage effective to maintain the oxygen evolution region of the aqueous solution, oxygen is generated at the working electrode, which can oxidise the metal atom, for example oxidise Ce(III) to Ce(IV). At the same time, protons are also rapidly generated, which can lower the local pH. The local pH of the aqueous solution during electrodeposition can be lowered to a more acidic pH compared to the initial pH of the aqueous solution, for example the local pH may be lowered to about pH 1 to about pH 3, for example less than about pH 3, 2.8, 2.6, 2.4, 2.2, or 2.0. The local pH of the aqueous solution during electrodeposition may be lower than the initial pH of the aqueous solution.

The generation of protons during the electrodeposition can provide a source for one or more labile ions (e.g., protons), which intersperse between the metal coordination polymer layers to form the electrostatic interaction between the one or more moieties of the optionally interrupted alkyl group of each R¹ of the organic linker of each metal coordination polymer layer to from the layered metal coordination polymer.

The electrodeposition may be performed at a suitable temperature, for example at a temperature of at least about 0, 5, 10, 15, 20, 25, 30, 40, 50, 70, 90, or 100° C. The electrodeposition may be performed at a temperature of less than about 100, 90, 70, 50, 40, 30, 25, 20, 15, 10, or 5° C. Combinations of any two or more of these upper and/or lower temperatures are also possible, for example between about 0° C. to about 100° C., about 10° C. to 60° C. or 25° C. to 50° C. In some embodiments, the electrodeposition may be performed at room temperature (e.g., 25° C.), however higher temperatures can accelerate the diffusivity and reaction rate of the formation of the metal coordination polymers.

The electrodeposition may be performed for a suitable time to form the metal coordination polymer, for example for a period of time of at least about 1, 2, 5, 10, 15, 20, 30, 60, or 90 minutes. The electrodeposition may be performed for a period of time of less than about 90, 60, 30, 20, 15, 10, 5, 2, or 1 minute. Combinations of any two or more of these upper and/or lower reaction times are possible, for example between about 1 minute to 90 minutes, about 10 minutes to 90 minutes, or about 30 minutes to about 90 minutes.

In some embodiments, the metal may be of low field strength at a lower oxidation state within a feasible working pH range of an aqueous solution comprising the metal atom and organic linker for forming the metal coordination polymer as described herein. The working pH range may be determined by Pourbaix diagrams. The oxidation state of the metal may increase upon oxidation in an acidic pH environment as described herein. In some embodiments an unsaturated metal hydroxide (M(OH)ₓⁿ⁺) may form in acidic pH at a higher oxidation state.

The architecture of the layered material may be changed by disassembling and reassembling the layered metal coordination polymer using different solvent systems. For example, in polar solvents such as water, the layered metal coordination polymer may preferentially exfoliate rather than change architecture. If less polar solvents are used, such as ethanol or other organic solvents, the layered metal coordination polymer may disassemble and then reassemble. The way in which the layered metal coordination polymer reassembles may be dependent upon a concentration of the layered metal coordination polymer, the type of solvent, use of a solvent system such as a gradient solvent system, evaporation rates, heating, cooling, pH, and introduction of groups that cause layering such as salts. Changing the architecture of the layered metal coordination polymer may allow the formation of different nanostructures from a single precursor layered material.

In some embodiments, the layered metal coordination polymer is disassembled in an organic solvent and reassembled from the organic solvent by evaporation. In some embodiments, the organic solvent is an alcohol, for example, methanol, ethanol, propanol, butanol, pentanol, etc., preferably ethanol. In some embodiments, the organic solvent is a polar aprotic solvent, e.g. dichloromethane, NMP, THF, acetates, acetone, DMF, acetonitrile, or DMSO, or an amine, for example triethylamine. In some embodiments, the organic solvent is an amine, for example triethylamine. In some embodiments, the organic solvent is acetone.

The concentration of metal coordination polymer disassembled in the organic solvent is limited by the maximal solubility of the metal coordination polymer in the organic solvent. In some embodiments, the concentration of metal coordination polymer disassembled in the organic solvent is at least about 1, 2, 4, 5, 10, 20, 50, 70, 90, 100, 110, or 120 M. In some embodiments, the concentration of metal coordination polymer disassembled in the organic solvent is less than about 120, 110, 100, 90, 70, 50, 20, 10, 5, 4, 2, or 1 M. Combinations of any two of these upper and/or lower concentrations can provide a range selection, for example between about 1 M to about 200 M, or about 4 M to about 120 M.

The evaporation of the organic solvent is performed at a temperature and vapour pressure limited by the maximal solubility of organic solvent in air. In some embodiments, the evaporation of the organic solvent is performed at a temperature of at least about −20, −15, −10, −5, 0, 5, 10, 15, 20, 30, 40. or 50° C. In some embodiments, the evaporation of the organic solvent is performed at a temperature less than about 50, 40, 30, 20, 15, 10, 5, 0, −5, −10, −15, or −20° C. or greater than about −20, −15, −10, −5, 0, 5, 10, 15, 20, 30, 40 or 50° C. Combinations of any two or more of these upper and/or lower evaporation temperatures are possible, for example between about −20° C. to about 40° C., or about −10° C. to about 25° C. In some embodiments, the evaporation of the organic solvent is performed at a vapour pressure of at least about 0.1, 0.2, 0.5, 0.7, 1, 2, 5, 7, 10, 15, or 20 kPa. In some embodiments, the evaporation of the organic solvent is performed at a vapour pressure of less than about 20, 15, 10, 7, 5, 2, 1, 0.7, 0.5, 0.2, or 0.1 kPa. Combinations of any two or more of these upper and/or lower vapour pressures are possible, for example between about 0.1 kPa to about 20 kPa, about 0.1 kPa to about 10 kPa, 0.5 kPa to about 10 kPa, or about 0.7 kPa to about 10 kPa. It will be appreciated that any single or range of vapour pressure and evaporation temperature can be combined. In some embodiments, the evaporation time may be at least at least about 1 min, 15 min, 30 min, 1, 2, 3, 4, 6, 8, 12, 18, 24, 48 or 72 hours. Combinations of these evaporation times are also possible for example between about 6 h and 72 h.

The layered metal coordination polymer may be exfoliated to obtain one or more metal coordination polymer layers. In some embodiments, the step of exfoliating the layered material comprises removing the interspersed labile ions within each layer. The removal of the interspersed labile ions may disrupt the electrostatic interaction between the metal coordination polymer layers to obtain one or more metal coordination polymer layers (e.g., a dispersion of metal coordination polymer layers). For example, the pH of a dispersion or solution of the layered metal coordination polymer may be increased to remove the interspersed labile ions (e.g., protons). Generally, the removal of the interspersed labile ions occurs at an edge of the layered metal coordination polymer, which weakens the Van der Waals forces that keep the layers stacked and this allows ingress of water or solvent molecules between adjacent sheets. The propagation front of ion removal and water or solvent ingress then proceeds from an edge towards an interior of the layered metal coordination polymer. In this way, in an embodiment, water or solvent ingress is responsible for exfoliation. In some embodiments, the layered metal coordination polymer is exfoliated by agitating in water. However, exfoliation is not limited to water or solvent ingress and may be facilitated by, for example, by adjusting a temperature, chemical environment, and so on.

In some embodiments, the exfoliation of the layered metal coordination polymer comprises removing the labile ions interspersed between each metal coordination polymer layer thereby disrupting the electrostatic interaction between the one or more moieties of the optionally interrupted alkyl group of each R¹ of the organic linker of each metal coordination polymer layer to obtain one or more metal coordination polymer layers.

Exfoliation may be performed by dispersing the metal coordination polymer in a suitable solvent (e.g., water or organic solvent), which may be additionally subjected to heating and/or agitation, such as stirring or (ultra) sonication. Exfoliation may be assisted through chemical means. Exfoliation may be aided by heating and/or sonication. Exfoliation may be performed using any solvent, for example water, alcohol, for example, methanol, ethanol, propanol, butanol, pentanol, etc.; preferably ethanol, a polar aprotic solvent, e.g. dichloromethane, NMP, THF, acetates, acetone, DMF, acetonitrile, or DMSO, or an amine, for example triethylamine.

In some embodiments, the exfoliation of the layered metal coordination polymer comprises dispersing the layered metal coordination polymer in water or an organic solvent and agitating to exfoliate the layered metal coordination polymer to obtain one or more metal coordination polymer layers. The layered metal coordination polymer may be agitated at a temperature of between about 5° C. to about 50° C., for example about room temperature. The layered metal coordination polymer may be agitated (e.g., by sonication) for a period of time effective to exfoliate the layered metal coordination polymer to obtain one or more metal coordination polymer layers. Suitable agitation times include for example between about 1 minute to about 72 hours, about 1 minute to about 60 minutes, or about 1 minute to about 20 minutes, to exfoliate the layered metal coordination polymer to obtain one or more metal coordination polymer layers.

In some embodiments, the disassembly of the layered metal coordination polymer in an organic solvent also exfoliates the layered metal coordination polymer. In some embodiments, the combined exfoliation and disassembly may be facilitated by similar polarity indices for the metal coordination polymer and solvent, e.g., organic or inorganic. In an embodiment, exfoliation may be facilitated by dissimilar polarity indices, which are within the range 1 to 10, for the metal coordination polymer and solvent. Similar polarity indices are, e.g., in the range ±2; dissimilar polarity indices are e.g., in the range ±3-9.

In some embodiments, the step of forming the layered metal coordination polymer comprised hydrothermal treatment of the aqueous solution comprising the metal atom and organic linker as described herein. The initial pH of the aqueous solution during hydrothermal treatment may be less than about 7. The hydrothermal treatment may be performed at the temperature range of between about 25° C. to about 200° C., or about 25° C. to about 100° C., e.g., less than about 100° C. Suitable techniques for hydrothermal treatment are known to the skilled person.

### Process for Preparing Nanostructures

The metal coordination polymers disclosed herein are relatively unstable precursors, which provide a platform for controllable disassembly to form a multitude of useful and/or previously unachievable nanoarchitectures, ranging from nanosheets that can be extremely thin, to diverse 2D and 3D nanostructures that can feature varying degrees of defects. Unexpectedly and advantageously, these diverse nanostructures can be obtained from a single metal coordination polymer precursor in a controlled manner. For example, exfoliation of metal coordination polymers can ultimately produce nanosheets, including metal oxides (MOs), that can be as thin as one unit cell and may be suitably diversified with, for example, useful transition metals. On the other hand, disassembly/reassembly of the metal coordination polymers under certain conditions can provide diverse 2D and 3D nanostructures based on the morphology of the reassembled metal coordination polymer. The assembly/reassembly process can be controlled by varying parameters such as solvent type, solute concentration, temperature, and time. Both of the initial steps of exfoliation and/or disassembly/reassembly are followed by removal of the metal coordination polymer coordinating organic linkers to transform the initial metal coordination polymer structures into the corresponding nanostructure, for example a holey metal oxide nanosheet.

Accordingly, the present disclosure provides, in one aspect, a method of forming a nanostructure, comprising providing a layered metal coordination polymer comprising two or more layers, each layer comprising metal atoms each coordinated to one or more organic linkers to form a metal coordination polymer, and removing at least some of the coordinating organic linkers to form the nanostructure.

In some embodiments, the method comprises providing a layered metal coordination polymer having a number of metal atoms that are stabilised by coordination to one or more organic linkers. In some embodiments, the layered metal coordination polymer comprises a number of reactive metal-based centres (i.e. reactive metal-based species), which are stabilised by coordination to one or more organic linkers. In some embodiments, the method may comprise removing at least some of the coordinating organic linkers to reveal the unstable metal-based species, which then convert to more stable metal-based species thereby forming the nanostructure. In some embodiments, the unstable metal-based species convert into one or more of a more stable intermediates before converting to a more stable metal-based species that forms the resulting nanostructure.

According to the method, at least some of the coordinating organic linkers are removed to form the nanostructure. In some embodiments, the step the removing at least some of the coordinating organic linkers to form the nanostructure comprises aging the layered metal coordination polymer.

As used herein, the term “aging” refers to the physical and/or chemical change of a material with respect to time, for example the metal coordination polymer is aged to form the nanostructure.

In some embodiments, the aging of the layered metal coordination polymer comprises heating the metal coordination polymer. For example, the layered metal coordination polymer may be heated to a temperature sufficient to decompose the organic linker to form the nanostructure. The sufficient temperature may be, for example, from 100° C. to 1000° C., preferably from 100° C. to 850° C., more preferably from 100° C. to 700° C. In some embodiments, the temperature may be at least about 50, 75, 100, 150, 200, 250, 300, 350, 400, 450, 500, 600, 700, 800, 900, or 1000° C. Combinations of these temperature values are also possible, for example between about 300° C. to about 400° C., e.g., about 350° C.

In some embodiments, at least some of the coordinating organic linkers are removed by pyrolysis of the layered metal coordination polymer. In some embodiments, the pyrolysis is low-temperature pyrolysis. In other embodiments, the pyrolysis is conducted at temperatures of at least 100, 150, 200 250, or 300° C.

Other possible treatments to form the nanostructure include X-ray irradiation, cold laser irradiation, gamma ray irradiation, neutron irradiation, and other suitable high-energy beam irradiation capable of forming the nanostructure from the metal coordination polymer.

In some embodiments, the removal at least some of the coordinating organic linkers to form the nanostructure comprises aging a solution comprising the layered metal coordination polymer. The solution comprising the metal coordination polymer may be sonicated or stirred during the aging step. Alternatively, the solution may be static during the aging step.

The aging of the solution may be at a basic pH (e.g., less acidic pH, for example by using solution concentrations up to 6.0 M NaOH). In some embodiments, the aging of the solution comprising the layered metal coordination polymer is at a basic pH of greater than pH 7, for example at least about pH 7, 8, 9, 10, 11, 12, 13, or 14, preferably pH 8. Combinations of these pH ranges are also possible, for example between about pH 7 to pH 14, or about pH 7 to about pH 10, e.g. pH about 8.

The aging of the solution at a basic pH may further comprise an agitating step. The agitating step may be performed at the same time as the aging of the solution at a basic pH. The aging of the solution at a basic pH may comprise raising the pH of the solution comprising the metal coordination polymer to the basic pH, for example by adding a suitable base e.g. sodium hydroxide. Alternatively, the solution may have its pH adjusted prior to adding the metal coordination polymer. In one embodiment, the step of removing one or more organic linkers comprises raising the pH of the solution. The solution may be agitated while raising the pH.

In some embodiments, the aging of the solution comprising the layered metal coordination polymer is for a period of time effective to form the nanostructure, for example at least about 1 min, 15 min, 30 min, 1 hour, 2 hours, 6 hours, 12 hours, 1 day, or 2 days. In some embodiments, the aging of the solution is for a period of time of between about 1 min to about 2 days, preferably between about 10 min to about 2 hours, e.g., about 30 min.

In some embodiments, the aging of the solution is at a temperature effective to form the nanostructure, for example at least about 1, 5, 10, 15, 20, 30, 40, 50, 70, or 100° C., and combinations thereof, for example between about 10° C. to about 50° C., preferably room temperature, for example about 25° C.

The aging may comprise a heating or calcining step. The heating or calcining step may comprise the heating or calcining of the solution comprising the metal coordination polymer. The heating or calcining of the solution may be a temperature of at least about 10, 20, 25, 30, 40, 50, 80, 100, 150, 200, 250, 300, 350, 400, 450, 500, 550, or 600° C. The heating or calcining of the solution may be at a temperature of less than about 600, 550, 500, 450, 400, 350, 300, 250, 200, 150, 100, 80, 50, 40, 30, 25, 20 or 10° C. Combinations of these heating or calcining temperatures are also possible, for example between about 10° C. to about 50° C., about 50° C. to about 600° C., about 100° C. to about 600° C. or about 200° C. to about 600° C.

The morphology of the nanostructure can vary depending on the heating or calcination rate. The heating or calcination rate may be at least about 0.01, 0.05, 0.1, 0.2, 0.3, 0.5, 1.0, 1.5, 2.0, 2.5, 3.0, 3.5, 4.0, or 5.0° C. min⁻¹. The heating or calcination rate may be less than about 5.0, 4.0, 3.5, 3.0, 2.5, 2.0, 1.5, 1.0, 0.5, 0.3, 0.2, 0.1, 0.05, or 0.01° C. min⁻¹. Combinations of these heating or calcination rates are also possible for example between about 0.1 to about 5° C. min⁻¹ or about 0.2 to about 3° C. min⁻¹. The heating or calcining may be performed for a suitable period of time, for example at least about 1 min, 15 min, 30 min, 1, 2, 3, 4, 8, 12, 18, 24, 48 or 72 hours.

The aged solution may be heated at a temperature up to the boiling point of the solution. In one embodiment, the aged solution may be heated at a temperature of between 100° C. to 300° C., for example about 200° C.

In some embodiments, the removal of at least some of the coordinating organic linkers destabilises the metal atom, which subsequently forms a stable nanostructure. In some embodiments, upon removal of at least some of the coordinating organic linkers, the metal coordination polymer converts (e.g., spontaneously or with application of heat, agitation, etc.) to form a stable nanostructure. In some embodiments, the morphology of the nanostructure is the same as the morphology of the metal coordination polymer.

As a result of the removal of at least some of the coordinating organic linkers, the resulting nanostructure may be a holey nanostructure. The step of removing at least some of the coordinating organic linkers to allow the reactive metal-based species (e.g., the uncoordinated metal atom centres/substructures) to form the more stable metal-based species forms the holey nanostructure. Accordingly, in some embodiments, the nanostructure is a holey oxide nanostructure, and the step of removing at least some of the coordinating organic ligands forms the holey nanostructure. In some embodiments, the nanostructure exhibits a fine and homogeneous pore network. The terms “nanostructure” and “holey nanostructure” are used interchangeably throughout this disclosure unless context makes it clear otherwise. For example, reference to a hole size is made in reference to a holey nanostructure.

The reactive metal-based species form part of the metal coordination polymer. The reactive metal-based species are stabilised by the coordinating organic linker so that the reactive metal-based species can exist in the “reactive” state while they are coordinated to the organic linker. In some embodiments, removal of the organic linker allows the unstable metal-based species, formed after removal of the organic linker, to adopt a more stable state (i.e., the more stable metal-based species). For example, the reactive metal-based species may be a multivalent metal, and in the reactive or unstable state the multivalent ion is in a first oxidation state and in the stable state the multivalent ion is in a second oxidation state. For example, the metal of the reactive metal-based species may have a first oxidation state when bound to the ligand and a second oxidation state when the ligand is removed. In some embodiments, the metal of the reactive metal-based species is a multivalent metal. The reactive metal-based species may include metals with two or more oxidation states. In some embodiments, the metal atom is selected from any one or more metal atoms (including ions) as described herein, including for example Ce, Cu, Mn, Fe, Ni, Zn, Ti, or Zr. In some embodiment, the metal atom is Ce, Ti, Zr. In some embodiments, the metal ion is Ce(IV), Ti(IV), Zr(IV).

In some embodiments, the method comprises providing a layered metal coordination polymer having a number of metal ions. In some embodiments, the metal ion is univalent or multivalent, preferably multivalent.

In some embodiments, the metal may be of low field strength at a lower oxidation state within a feasible working pH range for forming the metal coordination polymer. In some embodiments, the metal atom has an oxidation state that is capable of increasing upon oxidation in an acidic pH. The working pH range may be determined by Pourbaix diagrams. The oxidation state of the metal of the reactive metal centre may increase upon oxidation in acidic pH. In some embodiments an unsaturated metal hydroxide (M(OH)ₓⁿ⁺) may form in acidic pH at a higher oxidation state.

For example, in some embodiments, when the ligand is removed, the reactive metal-based species may form an unsaturated metal hydroxide as the unstable metal-based species, which then converts to a more stable metal oxide. Upon removal of at least some of the organic linkers, the reactive metal-based species may form an unstable metal oxide-based species. In aqueous systems, the unstable metal oxide-based species may include a hydroxide salt and a peroxide salt. In non-aqueous systems, other metal oxide-based species may be formed. As an example, when Ce is used as the metal of the reactive metal species while being coordinated to an organic linker, removal of the organic linker may promote the formation of Ce(OH)ₓ^{(4-x)+} as the unstable metal-based species, which in turn converts to CeO₂₋ₓ as the more stable metal-based species that forms the holey nanostructure.

In some embodiments, the transformation of a metal coordination polymer into a metal oxide is attributed to the replacement of weakly-bonded organic linkers by OH⁻/H₂O in aqueous solutions. For Ce-based coordination polymers, for example, in aqueous solution, the relatively high field strength of Ce⁴⁺ enhances its ability to form Ce(OH)₄, which readily converts to CeO₂₋ₓ upon drying. The conversion of the reactive metal-based species to more stable metal-based species may occur at room temperature (e.g., <˜35° C.). In some embodiments, a heating step is used to convert the reactive metal-based species to the more stable metal-based species.

In some embodiments, prior to removing at least some of the coordinating organic linkers to form the nanostructure, the layered metal coordination polymer is exfoliated to obtain a dispersion of metal coordination polymer layers. In some embodiments, the layers may be in the form of sheets of metal coordination polymer, and exfoliation results in the formation of a dispersion of discrete sheets.

In some embodiments, removing at least some of the organic linkers from the dispersion of discrete sheets may result in the formation of a dispersion of holey nanosheets. Alternatively, removing at least some of the organic linkers may form a species capable of forming the holes.

In an embodiment, the step of exfoliating the layered material and removing at least some of the coordinated organic linkers is performed at the same time. It should be noted that if the layered material is not exfoliated prior to the removal of the ligands, and irrespective of the mechanism used to form the holey nanostructure, each sheet may still be converted to a holey nanosheet, but the holes of each nanosheet may not be aligned with one another, which may give the appearance of a structure that does not appear “holey” but at the nano level is “holey”. The layered material does not have to be planar. In some embodiments the layered material may be in the form of a tube or rod. For example, the layers may wrap around a central axis of the layered material.

Exfoliation may be performed as described herein, for example using agitation. In some embodiments, the metal coordination polymer is dispersed in a suitable solvent (e.g. water) and agitated for a period of time effective to exfoliate the metal coordination polymer to obtain a dispersion of metal coordination polymer layers prior to removing at least some of the coordinating ligands to form the nanostructure. Suitable solvents may include water, polar protic solvents or polar aprotic solvents. Polar aprotic solvents may include dichloromethane, NMP, THF, acetates, acetone, DMF, acetonitrile, or DMSO. Polar protic solvents may include water, alcohol (e.g. ethanol and methanol) and carboxylic acids.

In some embodiments, the metal coordination polymer is agitated, preferably for a period of time of at least about 1 min, 2 min, 5 min, 8 min, 10 min, 15 min, 30 min, 1 hour, 2 hours, 6 hours, 12 hours, 1 day, or 2 days to exfoliate the metal coordination polymer to obtain a dispersion of metal coordination polymer layers and/or nanostructures. Combinations of these agitation times are also possible, for example, the metal coordination polymer is agitated for a period of time of between about 1 min to about 2 days, preferably between about 10 min to about 2 hours, e.g., about 30 min. to exfoliate the metal coordination polymer to obtain a dispersion of metal coordination polymer layers. Exfoliation may be assisted through chemical means. Exfoliation may also comprise heating and/or sonication. The exfoliation step may be at a basic pH, for example at a pH as described herein in relation to removing the one or more organic linkers. The exfoliation step and aging step may be performed at the same time.

In an embodiment, labile ions are interspersed between the metal coordination polymer layers as described herein. For example, when the organic linkers comprise terminal acidic groups, the labile ions may be protons of the acid and may be intercalated between the sheets. The intercalated protons may help to keep the layered material together. For example, the protons may act as weak crosslinking agents. In some embodiments, the step of exfoliating the layered material comprises removing the intercalated protons. For example, alkaline pH may be used to remove the intercalated protons. Generally, the removal of intercalated protons occurs at an edge of the layered material, which weakens the Van der Waals forces that keep the layers stacked and this allows ingress of water molecules between adjacent sheets. The propagation front of proton removal and water ingress then proceeds from an edge towards an interior of the layered structure. In this way, in an embodiment, water ingress is responsible for exfoliation. However, exfoliation is not limited to water ingress and may be facilitated by, for example, by adjusting the temperature, chemical environment, and so on.

The layered structure may have many different architectures. In some embodiments, prior to removal of at least some of the ligands, the structure (architecture) of the layered structure may change. During the change in layered structure, the reactive metal-based species may remain unchanged. The architecture of the layered material may be retained upon removal of the ligands to convert the reactive metal-based species into the more stable metal-based species. In some embodiments, the structure of the sheets does not change during the conversion of the reactive metal-based species to the more stable metal-based species. It should be appreciated that at an atomic level the structure may change but at a macro level an architecture of the nanostructure (e.g., nanosheet) does not change, for examples it remains as a 2D sheet. Changing the architecture of the layered material (i.e., a precursor material) may allow the formation of different nanostructures from a single precursor layered material.

In some embodiments, the architecture of the layered material may be changed by disassembling the layered material in different solvent systems as described herein. For example, in strongly polar solvents such as water, the layered material may preferentially exfoliate rather than change architecture. If less polar solvents are used, such as ethanol, the layers may disassemble and then reassemble. Accordingly, in some embodiments, prior to removing at least some of the coordinating ligands to form the nanostructure, the metal coordination polymer is disassembled in an organic solvent and reassembled from the organic solvent by evaporation to change the morphology of the metal coordination polymer prior to or during the removal of one or more organic linkers to form the nanostructure. In this way, tailored and unique nanostructure morphologies can be formed.

The way in which the layers reassemble may be dependent upon the concentration of the layered material, the type of solvent, use of a solvent system such as a gradient solvent system, evaporation rates, heating, cooling, pH, and introduction of groups that cause layering such as salts.

In some embodiments, the concentration of the metal coordination polymer dissolved in the organic solvent is limited by the maximal solubility of the metal coordination polymer in the organic solvent, preferably between about 4 M to about 120 M.

In some embodiments, the evaporation of the organic solvent is performed at a temperature and vapour pressure limited by the maximal solubility of organic solvent in air, preferably between about −20° C. to about 40° C., more preferably between about −10° C. to about 25° C., and at a vapour pressure of between about 0.1 kPa to about 10 kPa, preferably between about 0.5 kPa to about 8 kPa.

In some embodiments, the organic solvent is an alcohol, for example, methanol, ethanol, propanol, butanol, pentanol, etc.; preferably ethanol. In some embodiments, the organic solvent is a polar aprotic solvent, which may include dichloromethane, NMP, THF, acetates, acetone, DMF, acetonitrile, or DMSO. In some embodiments, the organic solvent is an amine, for example triethylamine. In some embodiments, the organic solvent is acetone.

The step of removing at least some of the coordinating organic linkers may comprise (i) increasing the affinity of the metals to form the more stable metal-based species, for example to convert to an oxidised form and/or (ii) reducing an affinity of the organic linkers to the reactive metal site. These may be achieved by changing the environment of the metal coordination polymer, for example by adjusting the solvent, a salt concentration, temperature, pH and/or introduction of agents that disrupt binding of the linker to the reactive metal-based species. In an embodiment, reducing the affinity of the linker to the reactive metal-based species comprises raising the pH of a mixture comprising the nanostructure. For example, when the linker comprises an acidic group, such as a carboxyl group, increasing the pH of a solution in which the metal coordination polymer is present deprotonates the carboxyl group to change the affinity of the carboxyl group by promoting the formation of reactive metal intermediates.

In some embodiments, reducing the affinity of the linker to the reactive metal-based species comprises heating the metal coordination polymer. A combination of processes may be used to reduce the affinity of the binding between reactive metal-based species and coordinating organic linkers, such as, for example, changing the pH and the temperature.

In some embodiments, lability deriving from weak electrostatic bonding between the cation (e.g., metal ion) and organic linker (e.g., an organic acid-comprising organic linker) in unstable coordination polymers provides a valuable platform for easy and controllable destruction/reconstruction of coordination polymer crystallites to form previously unobserved nanostructures e.g., CeO₂, nanostructures.

The metal coordination polymer used to prepare the nanostructures may be a metal coordination polymer as described herein, or a metal coordination polymer prepared by the process described herein.

### Nanostructures

The nanostructures that can be produced by the present method are diverse. In some embodiments, the present disclosure provides a nanostructure. In some embodiments, the nanostructure exhibits a fine and homogeneous pore network. In some embodiments, the nanostructures are bulk nanostructures.

The morphology of the nanostructure may be sheet-like, hollow, holey, cubic, rod-like, polyhedral, spherical or semi-spherical, rounded or semi-rounded, angular, and irregular morphology, tubular, dumbbell-like, rhombohedral, honeycomb, needle-like, bundle-like, wafer-like, fibres, flower-like, and so forth, and may also include 2D and/or 3D scaffold structures comprising the same. The morphology of the nanostructure may correspond to the morphology of the layered metal coordination polymer used to prepare the nanostructure.

In some embodiments, the nanostructure is polycrystalline. In some embodiments, the nanostructure is solid and/or hollow. The hollow nanostructure may be faceted. The nanostructure may be a holey nanostructure. The step of removing at least some of the organic linkers allow the reactive metal centres to form the holey nanostructure.

In some embodiments, the nanostructure is a metal oxide. The metal oxide may be an oxide of any metal described herein in relation to the metal coordination polymer.

In some embodiments, the nanostructure is a nanosheet or a nanolayer. For example, the metal coordination polymer is exfoliated to form one or more metal coordination polymer layers which are then aged to remove one or more organic linkers therefrom to form a nanosheet. The nanosheet may be solid or hollow. In one embodiment, the nanosheet is a metal oxide.

In one embodiment, the nanosheet is a holey nanosheet. In one embodiment, the nanosheet is a holey metal oxide nanosheet, for example a holey CeO₂₋ₓ nanosheet, wherein x can vary between 0 and 0.9, 0 and 0.8, 0 and 0.7, 0 and 0.6, and 0 and 0.5. The holey metal oxide nanosheet may be a holey FCO nanosheet, a holey NCO nanosheet or a holey ZCO nanosheet.

In one embodiment, the nanostructure is a bulk metal oxide nanostructure. The bulk metal oxide nanostructure may be porous. The bulk metal oxide nanostructure may be 1D, 2D or 3D. The bulk nanostructure may be solid or hollow.

In one embodiment, the nanostructure is a holey metal oxide nanosheet. The nanosheet may have an average hole size of at least about 1, 2, 3, 4, 5, 8, 10, 12, 14, 18, or 20 nm. The nanosheet may have an average hole size of less than about 20, 18, 14, 12, 8, 5, 4, 3, 2, or 1 nm. Combinations of any two or more of these upper and/or lower diameters are also possible, for example between about 2 nm to about 20 nm, for example 2 nm to about 14 nm. The hole size can be measured using transmission electron microscopy.

In some embodiments, the nanostructure is a metal oxide nanosheet having a concentration of point defects (e.g. cation vacancies and/or anion vacancies). The concentration of point defects may depend on the type of metal oxide nanostructure and/or the morphology of the metal coordination polymer used to prepare the metal oxide nanostructure. In some embodiments, the metal oxide nanosheet has a defect concentration of at least about 1, 2, 5, 10, 12, 14, 18, 20, 25, 30, 35 or 40 atomic %. In some embodiments, the metal oxide nanosheet has a defect concentration of less than about 40, 35, 30, 25, 20, 18, 14, 12, 10, 5, 2, or 1 atomic %. Combinations of any two or more of these upper and/or lower defect concentrations are also possible, for example between about 1 to about 30 atomic %, for example 18 to about 30 atomic %.

The nanostructures may have a BET specific surface area. The specific surface area may be at least about 25, 50, 75, 85, 95, 100, 200, 500 or 1000 m²/g. The specific surface area may be less than about 1000, 500, 200, 100, 95, 85, 75, 50 or 25 m²/g. The specific surface area may be at least about 70, 75, 80, 85, 90, 95, 100 m²/g. Combinations of any two or more of these upper and/or lower specific surface areas are also possible, for example between about 75 to about 1000 m²/g.

The nanostructures may be polycrystalline. The polycrystalline nanostructures may comprise one or more crystallites. The average crystallite size may be less than 100, 80, 60, 50, 40, 30, 20, 15, 10, or 5 nm. The average crystallite size nay be between about 1 nm to about 20 nm.

The nanostructure may be a nanolayer, for example a nanosheet. The nanosheet may be a holey nanosheet. The nanosheet may have a certain thickness across the layer or sheet (e.g. cross-section distance), referred to as an axial thickness along the c-axis of the sheet or layer. In some embodiments, the nanosheet may have an axial thickness along the c-axis of less than 100, 70, 50, 20, 15, 10, 8, 6, 4, 2, or 1 nm, for example less than 20, 15, 12, 10, 8, 5, 2, or 1 nm. Combinations of any two or more of these upper and/or lower thickness are also possible, for example between about 1 nm to about 100 nm, about 1 nm to about 50 nm, or about 1 nm to about 20 nm.

The nanosheet may be a holey nanosheet, wherein the average diameter of the holes may be at least about 1, 2, 3, 4, 5, 6, 7, 8, 9, 10, 15, 20, 50, 80, or 100 nm. The nanosheet may be a holey nanosheet, wherein the average diameter of the holes may be less than about 100, 80, 50, 20, 15, 10, 9, 8, 7, 6 5, 4, 3, 2, or 1 nm. Combinations of any two or more of these upper and/or lower average pore sizes are also possible, for example, between about 1 nm to about 50 nm, or about 2 nm to about 14 nm.

In one embodiment, there is provided a holey ceria nanosheet. The holey ceria nanosheet may have an axial thickness of less than 100, 70, 50, 20, 15, 10, 8, 6, 4, 2, or 1 nm, for example less than 20, 15, 12, 10, 8, 5, 2, or 1 nm. Combinations of any two or more of these upper and/or lower thickness are also possible, for example between about 1 nm to about 100 nm, about 1 nm to about 50 nm, or about 1 nm to about 20 nm.

In one embodiment, the nanosheet have an axial thickness of about 1.1, 2.2, 5.5 or 11 nm, wherein the thickness is proportional to the thickness of one unit cell of the metal coordination polymer. In some embodiments, the nanosheet may be about 1, 2, 3, 4, 5, 6, 7, 8, 9, or 10 unit cells thick. The thickness may be measured using scanning electron microscopy or atomic force microscopy (AFM).

In some embodiments, the nanostructure may comprise of multiple individual layers, each layer stacked to form individual nanolayers. In some embodiments the individual nanolayers may stack to form a bulk nanostructure. In some embodiments, the nanostructure is a metal oxide nanosheet, wherein two or more metal oxide nanosheets are stacked to form a bulk metal oxide nanostructure. In some embodiments, the nanostructure is a holey metal oxide nanosheet, and a plurality of nanosheets are stacked to form a stacked nanostructure.

In some embodiments, the morphology of the nanostructure is the same as the morphology of the metal coordination polymer used to prepare the nanostructure. For example, if the metal coordination polymer is a hollow nanotube, following removal of one or more organic linkers described herein, the resulting nanostructure may also be a hollow nanotube.

### Heterojunction Nanostructures

The metal coordination polymers described herein can also be used to prepare heterojunction nanostructures, where one or more adsorbate species may be adsorbed onto the surface of the nanostructure. The adsorbate species may be a second metal species. Adsorbing a second metal species onto the surface may form a metal-functionalised nanostructure. The nanostructure may act as a template.

The adsorbate species be adsorbed onto the surface by dispersing the nanostructure in a solution or dispersion comprising the adsorbate species. The adsorbate species may be adsorbed in the holes and on the surface. Where the nanostructure is a holey nanostructure, a surface charge of the holey nanostructure may provide attractive forces to allow adsorption of the adsorbate species. For example, the holey nanostructure may have a negative zeta potential, and positive metal ions may be attracted to and adsorb evenly over the surface of the holey nanostructure.

In some embodiments, one or more adsorbate species are adsorbed onto the surface of the nanostructure to form one or more heterojunctions on the surface of the nanostructure.

In some embodiments, the one or more species are adsorbed onto the surfaces of the nanostructure by removing at least some of the organic linkers in the presence of the adsorbate species.

The adsorbate species may be aged in the presence of the metal coordination polymer. For example, the adsorbate species may be added to the aged solution comprising the metal coordination polymer or may be aged with the solution comprising the metal coordination polymer. In some embodiments, the adsorbate species is mixed with the aged solution comprising the metal coordination polymer when the solution is at a pH of between about pH 3 to about pH 7.

Preferably, the adsorbate species comprise one or more metal atoms that are different from the metal atoms of the metal coordination polymer. Alternatively, adsorbate species may be the same metal as the metal atom of the metal coordination polymer, but with a different valency.

The adsorbate species may be in an ionic form. More preferably, the adsorbate species are a metal, non-metal, semimetal, or metalloid, or a combination thereof, including elemental, ionic forms, oxides, or non-oxides thereof, preferably including those of S, C, N, C, As, Te, O, Se, P, Mn, Fe, Ni, Cu, Zn, Mo, and Ru, including mixtures thereof. The adsorbate species may also be, for example, a metal-based species, which is oxidised following adsorption onto the surface of the nanostructure. Alternatively, once adsorbed, the metal species may be reduced to the elemental M⁰ form. The nanostructure may be doped with one or more adsorbate species described herein.

In an embodiment, once the adsorbate species has been adsorbed onto the surface of the nanostructure, the nanostructure is subject to oxidising conditions including calcining, chemical oxidation with an oxidising agent. The adsorbate species may help to alter catalytic, electropotential, hole size (if nanostructure is a holey nanostructure), and/or selectivity properties of the nanostructure, e.g. to allow passage of selective species through the holes of a holey nanosheet.

In some embodiments, a solution comprising the metal coordination polymer is aged in the presence of one or more adsorbate species to form one or more heterojunctions on the surface of the nanostructure. For example, the one or more adsorbate species may be dissolved or suspended in the solution comprising the metal coordination polymer which is aged to form one or more heterojunctions on the surface of the nanostructure. Alternatively, the adsorbate species is part of the organic solvent used to prepare the solution comprising the metal coordination polymer. For example, the metal coordination polymer may be dissembled in an organosulfur solvent (e.g. DMSO), which is then reassembled and aged to form a mixed metal oxide/sulfide nanostructure.

In some embodiments, the nanostructure may be a metal oxide, metal sulfide, metal arsenide, metal selenide, metal telluride, metal phosphide, metal nitride, or metal carbide, or a mixtures thereof. In one embodiment, the metal coordination polymers may be used as a precursor to form hybrid nanostructures comprising sulfur and/or carbon. In one embodiment, the nanostructure is a mixed ceria sulfide carbide.

Also disclosed is a nanostructure comprising a holey nanosheet having a metal oxide. The nanosheet may have a thickness of less than 30 unit cells. The holes may result from removing ligands bound to reactive metal-based species that form the metal oxide.

A thickness of the nanosheet may be less than 5 unit cells, such as 2 unit cells. The thickness may be 1 unit cell thick. A thickness of the nanosheet in nanometres (nm) may depend on the size of the unit cell and the number of unit cells. In an embodiment, the nanostructure has a defect concentration of approximately 18-30 at %.

The nanosheets may be formed from a metal coordination polymer as described herein. The nanosheets may have the same morphology and/or structure as the metal coordination polymer. The nanostructure may comprise a plurality of the holey nanosheets. For example, the plurality of holey nanosheets may be stacked to form a stacked structure. When the nanostructure has a plurality of holey nanosheets, the holes of adjacent sheets may be aligned with one another. However, in some embodiments, the holes of adjacent sheets may not be aligned with one another. When the holes of adjacent sheets are not aligned with one another, the nanostructure may not appear as having holes at a macro level.

The metal oxide may be an oxide of a multivalent metal. The metal of the metal oxide may be a metal that is multivalent. The metal of the metal oxide may have a high coordination number. The metal oxide may include oxides of Ce, Cu, Mn, Fe, Ni, Ti, Zr, and Zn. A surface of the nanosheet may be decorated with a second metal-based species (viz., heterojunction, plasma resonance). The second metal-based species may include a mixture of metal-based species, such as a mixture of having two or more metal-based species. The second metal-based may be in ionic, metallic or/or oxide form. Electropotential properties of the nanosheet may be adjusted through the inclusion of the second metal.

The nanostructure may be a mixed cerium oxide. The mixed cerium oxide may comprise one or more oxides of Cu, Mn, Fe, Ni, Ti, Zr and Zn. The mixed cerium oxide may be FCO, NCO or ZCO.

The holey nanosheets may have a surface charge of less than zero (0) mv. In some embodiments, the holey nanosheets may have a zeta potential of less than about 0, −5, −10, −15, −20, −25, −30, −40, −50, −80 or −100 mv. Combinations of these zeta potentials are also possible, for example between about −10 mV to about −40 mv.

In some embodiments, once the adsorbate species (e.g. second metal-based species) adsorbed onto the surface of the nanostructure, the adsorbate species is subject to structural transformation by O, N, S, Se, or Te.

### Catalyst Compositions

The nanostructures described herein have one or more catalytic properties. Accordingly, in one aspect there is provided a catalyst composition comprising a nanostructure according to any embodiments or examples thereof as described herein. The nanostructures can be used as a catalyst. In one embodiment, there is provided a method of catalysing a reaction using a nanostructure or catalyst compositions thereof according to any embodiments or examples described herein. The reaction may be an oxidation reaction. The nanostructures or catalyst compositions thereof may catalyse the oxidation of one or more reactants. The reaction may comprise the oxidation of one or more contaminants or pollutants present in an aqueous or gaseous environment.

In some embodiments, there is provided a method of purifying a gaseous stream or atmosphere (e.g. air) by contacting the gaseous stream or atmosphere with a nanostructure or catalyst composition thereof according to any embodiments or examples described herein, wherein one or more contaminants or pollutants present in the gaseous stream or atmosphere are catalytically reacted (e.g. oxidised) upon contact with the nanostructure or composition thereof. The gaseous stream or atmosphere may comprise carbon monoxide. The nanostructure or catalyst composition thereof may oxidise carbon monoxide to carbon dioxide. In one embodiment, there is provided a method of purifying a gaseous stream or atmosphere comprising carbon monoxide, the method comprising contacting the gaseous stream or atmosphere with a nanostructure or catalyst composition thereof according to any embodiments or examples described herein to oxidise the carbon monoxide to carbon dioxide. The gaseous stream or atmosphere may be an exhaust stream (e.g. industrial flue gas or car exhaust).

In some embodiments, the nanostructures or compositions comprising the same may achieve complete CO oxidation (e.g. to CO₂) (i.e. 100% CO oxidation) at a temperature of less than about 200, 190, 180, 170, 160, 150, 140, 130 120, 110, 100, 90 or 80° C., for example between about 80° C. to about 200° C. In some embodiments, the nanostructures or compositions comprising the same may achieve 50% CO oxidation to CO₂ at a temperature of less than about 200, 190, 180, 170, 160, 150, 140, 130 120, 110, 100, 90 or 80° C., for example between about 80° C. to about 200° C.

In some embodiments, the nanostructures or compositions thereof has a CO to CO₂ conversion rate at 400° C. of at least about 1, 2, 5, 7, 10, 12, 15, or 20 mol g⁻¹s⁻¹ and/or a CO to CO₂ turnover frequency (TOF) of at least about 1, 2, 3, 4 or 5×10⁻³ mol mol⁻¹s⁻¹. Combinations of these catalytic properties are also possible, for example in some embodiments, the nanostructures or compositions thereof has a CO to CO₂ conversion rate at 400° C. of between about 1 to 20 mol g⁻¹s⁻¹ and/or a CO to CO₂ turnover frequency (TOF) of between about 1 to about 5×10⁻³ mol mol⁻¹s⁻¹. In some embodiments, the nanostructures or compositions thereof has a CO to CO₂ conversion rate according to the performance provided in FIG. 64.

The nanostructures or compositions thereof may be used to purify an aqueous stream (e.g. water), by contacting the aqueous stream with a nanostructure or catalyst composition thereof according to any embodiments or examples described herein, wherein one or more contaminants or pollutants present in the aqueous stream are catalytically degraded (e.g. oxidised) upon contact with the nanostructure or composition thereof.

The catalyst composition may comprise or consist of the nanostructure and optionally one or more additives. Suitable additives may include one or more inert materials, for example binders and fillers, and/or one or more catalytic promotors to enhance catalytic activity.

The catalyst composition may be provided as any suitable composition. In one embodiment the catalyst composition may be a coating composition. The coating composition may be applied to a surface or substrate, for example quartz wool. Additional additives, such as binders, may facilitate coating of the catalyst composition to a surface. The catalyst composition or coating thereof may be provided as a partial coating or a complete layer on a surface. The catalyst composition may be deposited on a surface by brush coating, painting, slurry spraying, spray pyrolysis, dip coating, ink printing, sputtering, chemical or physical vapour deposition techniques, electroplating, screen printing, or tape casting.

In an embodiment, the nanostructure loading in the catalyst composition may be less than 90 wt. %, 80 wt. %, 70 wt. %, 60 wt. %, 50 wt. %, 40 wt. %, 30 wt. %, 20 wt. %, 18 wt. %, 16 wt. %, 14 wt. %, 12 wt. %, 10 wt. %, 8 wt. %, 6 wt. %, 4 wt. %, or 2 wt. %. The catalyst loading may be at least 1 wt. %, 3 wt. %, 5 wt. %, 7 wt. %, 9 wt. %, 11 wt. %, 13 wt. %, 15 wt. %, 17 wt. % 10 wt. %, 20 wt. %, 30 wt. %, 40 wt. %, 50 wt. %, 60 wt. %, 70 wt. %, 80 wt. % or 90 wt. %. In one embodiment, the catalyst consists of the nanostructure.

The present disclosure may also be defined with reference to one or more of the following numbered paragraphs:
1. A method of forming a nanostructure, comprising:

providing a metal coordination polymer having a number of reactive metal-based species that are coordinated to one or more ligands; and

removing at least some of the coordinated ligands to allow the reactive metal-based species to form a more stable metal-based species thereby forming the nanostructure.

2. The method according to paragraph 1, wherein removing at least some of the ligands comprises raising the pH of a mixture comprising the nanostructure.
3. The method according to paragraphs 1 or 2, wherein removing at least some of the ligands comprises heating the metal coordination polymer.
4. The method according to any one of paragraphs 1 to 3, wherein the ligands have only one binding site.
5. The method according to any one of paragraphs 1 to 4, wherein the ligands comprise a carboxyl group.
6. The method according to any one of paragraphs 1 to 5, wherein the metal coordination polymer forms a sheet, wherein a plurality of sheets can assemble to form a layered material.
7. The method according to paragraph 6, wherein, prior to removing at least some of the coordinated ligands, the layered material is exfoliated to form a dispersion of discrete sheets.
8. The method according to paragraph 7, wherein the step of exfoliating the layered material and removing at least some of the coordinated ligands is performed at the same time.
9. The method according to paragraph 7 or 8, wherein labile ions are intercalated between the stacked sheets, and the step of exfoliating the layered material comprises removing the intercalated labile ions.
10. The method according to any one of paragraphs 6 to 9, further comprising changing a structure of the layered material prior to removing at least some of the coordinated ligands, wherein the reactive metal-based species remain unchanged during changing the structure of the layered material.
11. The method according to any one of paragraphs 1 to 10, wherein the step of providing the metal coordination polymer includes forming the metal coordination polymer, wherein forming the metal coordination polymer comprises mixing a first metal atom and a ligand, wherein the metal of the first metal atom is the same as the metal in the reactive metal-based species.
12. The method according to paragraph 11, wherein the step of forming the metal coordination polymer comprises electrodeposition.
13. The method according to paragraph 11, wherein the step of forming the layered material comprises heating the solution of the metal atom and ligand.
14. The method according to any one of paragraphs 1 to 13, wherein the metal of the reactive metal-based species is multivalent.
15. The method according to any one of paragraphs 1 to 14, wherein the reactive metal-based species forms an unstable metal oxide-based species upon removal of the ligands.
16. The method according to paragraph 15, wherein the unstable metal oxide-based species converts to a more stable metal oxide.
17. The method according to any one of paragraphs 1 to 16, further comprising adsorbing a second metal-based species onto a surface of the nanostructure.
18. The method according to paragraph 17, wherein the second metal-based species is adsorbed onto the surface of the nanostructure by removing at least some of the coordinated ligands in the presence of the second metal.
19. The method according to paragraph 17 or 18, wherein the second metal-based is different to the more stable metal-based species.
20. The method according to any one of paragraphs 17 to 19, wherein the second metal-based species is in a form that includes ionic forms, and oxides and non-oxides of metals, non-metals, semi-metals and/or metalloids.
21. The method according to any one of paragraphs 17 to 20, wherein the second metal-based species includes Cu, Ni, Fe and Zn.
22. The method according to any one of paragraphs 17 to 21, wherein, once the second metal-based species has been adsorbed onto the surface of the nanostructure, the second metal-based species is subject to oxidation.
23. The method according to any one of paragraphs 1 to 22, wherein the reactive metal-based species form the more stable metal-based species at room temperature.
24. The method according to any one of paragraphs 1 to 23, wherein a structure of the metal coordination polymer does not change during the formation of the more stable metal-based species.
25. The method according to any one of paragraphs 1 to 24, wherein the nanostructure is a holey nanostructure, and the step of removing at least some of the coordinated ligands to allow the reactive metal-based species to form the more stable metal-based species forms the holey nanostructure.
26. The method according to anyone of paragraphs 1 to 25, wherein the nanostructure is polycrystalline.
27. The method according to any one of paragraphs 1 to 26, wherein the nanostructure is solid and/or hollow.
28. A nanostructure prepared using the method according to any one of paragraphs 1 to 27.
29. A nanostructure comprising a nanosheet having a metal oxide, wherein the nanosheet result from removing ligands that are bound to reactive metal-based species that go on to form the metal oxide.
30. The nanostructure according to paragraph 29, wherein a thickness of the nanosheet is less than 5 unit-cells
31. The nanostructure according to paragraph 30, wherein the thickness is 2 unit-cells or less.
32. The nanostructure according to any one of paragraphs 29 to 31, wherein the metal oxide includes oxides of Ce.
33. The nanostructure according to any one of paragraphs 29 to 32, wherein the nanosheet is a holey nanosheet.
34. The nanostructure according to paragraph 33, wherein a diameter of the holes ranges from about 2 nm-14 nm.
35. The nanostructure according to any one of paragraphs 29 to 34, wherein the nanosheet has a defect concentration of approximately 18-30 at %.
36. The nanostructure according to any one of paragraphs 29 to 35, comprising a plurality of the nanosheets, the plurality of nanosheets being stacked to form a stacked structure.
37. The nanostructure according to any one of paragraphs 29 to 36, wherein the nanostructure is formed from a metal coordination polymer precursor, and a structure of nanostructure is the same as the metal coordination polymer precursor.
38. The nanostructure according to any one of paragraphs 29 to 37, wherein the nanostructure is polycrystalline.
39. The nanostructure according to any one of paragraphs 29 to 38, wherein the nanostructure is solid and/or hollow.
40. A catalyst comprising the nanostructure of any one of paragraphs 28 to 39.
41. The catalyst according to paragraph 40, wherein the catalyst is a photocatalyst or an oxidation catalyst.
42. Use of the holey nanostructure of any one of paragraphs 28 to 39 as a catalyst.

### EXAMPLES

The present disclosure is further described by the following examples. It is to be understood that the following description is for the purpose of describing particular embodiments only and is not intended to be limiting with respect to the above description.

### Materials and Methods

### Transmission Electron Microscopy (TEM)

Dry powder of the specimens was suspended in water and drop-cast onto a carbon-supported Cu grid followed by air-drying at room temperature. The prepared samples were used for TEM, scanning transmission electron microscopy (STEM), high angle annular dark-field (HAADF), energy dispersive spectroscopy (EDS), and electron energy loss spectroscopy (EELS) analysis. High-resolution transmission TEM (HRTEM) images and EDS analysis of the nanostructures were taken by a Philips CM 200 microscope (Eindhoven, the Netherlands), while HAADF images, and EELS analysis were conducted by JEOL JEM-ARM200F microscope (Tokyo, Japan). Both machines were operated at an accelerating voltage of 200 kV. Additionally, the beam flux was reduced to very low values of ˜15 pA to minimize the beam damage effects. Finally, spectroscopy was conducted using spectrum imaging mode with sub-pixel scanning operative. This procedure ensured that at all times during the acquisition, the beam was moving, and the local flounce was minimized. Also, to avoid the beam damage on the sample during EELS measurement, the sample was cooled down to liquid nitrogen temperature.

### Scanning Electron Microscopy (SEM)

Scanning electron microscopy images were obtained by SEM (FEI Nova NanoSEM; secondary electron emission; accelerating voltage 5 kV, Hillsboro, Oreg., USA).

### X-Ray Photoelectron Spectroscopy (XPS)

Surface analysis of the samples was carried out using a Thermo Fisher Scientific ESCALAB 250Xi spectrometer (Loughborough, Leicestershire, UK) equipped with a monochromatic Al Kα source (1486.6 eV) hemispherical analyzer. The XPS samples were prepared by drop-casting an aqueous suspension of the nanostructure on the substrates followed by air-drying at room temperature. The pressure in the analysis chamber was maintained <8-10 mbar during the acquisition of the XPS data. All binding energies are referenced to the C1s signal corrected to 285 eV and the spectra were fitted using a convolution of Lorentzian and Gaussian profiles.

### X-Ray Diffraction (XRD)

Mineralogical data for the nanostructures were obtained using a Philips X'Pert Multipurpose X-ray diffractometer (Almelo, Netherlands) with CuKα radiation of [0.15405 nm], 2θ of 20°-80°, step size of 0.02°, and scanning speed of 5.5° 2θ/min. The peaks were analyzed using X'Pert High Score Plus software (Malvern, UK).

### Neutron Diffraction (ND)

Neutron diffraction patterns for structural analysis were collected on the high-intensity powder diffractometer Wombat, installed on the Open Pool Australian Light-water (OPAL) reactor at the Australian Nuclear Science and Technology Organisation (ANSTO). Two datasets with 1.63 Å and 2.41 Å were collected based on a CaAlNaF3 standard sample.

### Raman Spectroscopy (Raman)

Raman data were collected using a Renishaw inVia confocal Raman microscope (Gloucestershire, UK) equipped with a helium-neon green laser (514 nm) and diffraction grating of 1800 g/mm. All Raman data were recorded at laser power of 35 mW and a spot size of ˜1.5 μm. The data analysis was performed using Renishaw WiRE 4.4 software and the spectra were calibrated with respected to the silicon peak located at ˜520 cm-1.

### Thermogravimetric Analysis (TGA)

The decompositions of the Ce-CPs were assessed by using thermogravimetric analysis (TGA; TA Instruments, Q5000, 20°−1000° C., 10° C./min heating rate at different atmospheres of nitrogen and air.

### Fourier Transform Infrared Spectroscopy (FTIR)

ATR-FTIR; Spotlight 400 FTIR, PerkinElmer (Waltham, Mass., USA) within the wavelength of 400-4000 cm-1 was used to determine the chemical species present in the Ce-CP.

### Ab-Initio Molecular Dynamics (MD) Simulation

Density functional calculations were performed based on augmented plane wave pseudopotentials with Perdew-Burke-Ernzerhof functional as implemented in the VASP code [Comput. Mater. Sci. 1996, 6, 15]. For the electronic setting, a fine Monkhorst-Pack k-point grid with a spacing of 0.05 Å-1 and an energy cut-off of 520 eV were used. To find the ground state configuration, we ran a quenching ab initio molecular dynamics simulations was run based on a micro-canonical ensemble with a target temperature of 20 K with steps of 0.1 fs for 10 ps. Full geometry optimization was then carried out on the equilibrated structure, with convergence criteria for the energy and forces of 10-6 eV and 10-2 eV/A, respectively. The final geometry optimization run was conducted with Van der Waals correction (vdw-DFT) based on Michaelides's approach applied [Phys. Rev. Lett. 2004, 92; Phys. Rev. B 2011, 83, 195131].

### Atomic Force Microscopy (AFM)

The thickness of nanosheets was measured by atomic force microscopy (AFM; Bruker Dimension Icon SPM, PeakForce Tapping mode). A ScanAsyst-Air probe (Bruker AFM probes) was installed in the AFM holder and used for all measurements. The samples were printed on either glass or silicon substrate by applying a slight vacuum. The pixel resolution was 512 samples/line. A slow scan rate of 0.195 Hz was used to ensure accuracy. The peak force was minimized to avoid sample deformation and the feedback gain settings were optimized accordingly. The thicknesses of the thin films were determined using height profile with line scanning.

### Kelvin Probe Force Microscopy (KPFM)

Amplitude modulated KPFM (AM-KPFM) measurement were performed using the Bruker Dimension ICON SPM with a Nanoscope V controller. A platinum-iridium coated AFM tip (SCM-PIT-V2, Bruker AFM probes) was used to scan the surface. The probe was firstly installed on a cantilever holder, and the laser was aligned onto the back of the cantilever. Then the probe was tuned near its resonance frequency with a small offset to the right-hand side of the resonance curve (typically for normal tapping mode image, the left side of the resonance curve is tuned, which makes the interaction force on the surface slightly repulsive. However, it was found for KPFM measurements, the offset to the right-hand side provided better results in selected specimens). The oscillation amplitude was kept around 30 to 40 nm, depending on the specimen. The amplitude setpoint and gains were adjusted accordingly for each specimen. The scan rate was around 0.3 to 0.4 Hz with a scan size of 10 μms and 512 samples per line as the resolution. The scan setting included: Amplitude setpoint=172 mV, gains=1.1, scan rate=0.326 Hz. Further, the operating parameters were as follows: The lift height was fixed at 50 nm for the specimens to avoid any influence from surface topography (sometimes a smaller lift height of 30 nm is used when scanning smaller areas). The drive2 amplitude of the AC bias applied to the tip during the lift pass was set to 500 mV with a 170° phase angle. Also, for calibration tests, which were done before and after measuring the specimen, the same AFM tip was also measured against a freshly cleaved HOPG sample and/or a pre-calibrated TiO2 on a silicon reference sample. This calibration was important to determine the work function of the platinum tip, which can vary significantly from tip to tip.

### Photoluminescence (PL) Spectroscopy

PL was done using a spectrofluorophotometer (RF-5301PC, Shimadzu, Kyoto, Japan). The samples were used as free-standing stacked nanosheets.

### Zetapotential Measurement

The zeta potential also was determined using Zetasizer Nano ZS (Malvern Instruments, 4 mW He—Ne laser, 633 nm). For this work, the CeO₂₋ₓ and heterojunction nanostructures were suspended in 3 mL of deionized water at a concentration of 20 μg/mL using 10 mL individual glass tubes. The suspensions were sonicated for 2 min prior to running the measurement.

### First-Principles Calculations Details

First-principles calculations based on density functional theory (DFT) [Rev. Mod. Phys. 2017, 89, 035003] were performed to simulate and analyze the band structure differences between ceria nanosheets, the corresponding bulk system, and 0D/2D heterostructures. The PBEsol functional as implemented in the VASP software was used. A “Hubbard-U” scheme with U=3 eV is employed for a better treatment of the localized Ce 4f, Fe 3d, Ni 3d, and Zn 3d electronic orbitals. The “projector augmented wave” method was used to represent the ionic cores by considering the following electrons as valence: Ce 4f, 5d, 6s, and 4d; Fe 3d and 4s; Ni 3d and 4s; Zn 3d and 4s; and O 2s and 2p. Wave functions are represented in a plane-wave basis truncated at 650 eV. For integrations within the Brillouin zone we employ Monkhorst-Pack k-point grids with a density equivalent to that of 16×16×16 in the fluorite CeO₂ unit cell. Geometry relaxations are performed with a conjugate-gradient algorithm that allows for simulation cell shape and volume variations. The relaxations are halted when the forces in the atoms fall all below 0.01 eV·Å-1. By using these technical parameters we obtain zero-temperature energies that are converged to within 0.5 meV per formula unit. In order to estimate accurate electronic densities of states and band gaps, we employ the hybrid HSE06 exchange-correlation functional to perform single-point calculations on the equilibrium geometries determined at the PBEsol+U level.

### Photocatalytic Activity Test

The photocatalytic activity of the nanostructures was evaluated by analysis of photodegradation of methylene blue (MB, M9140, dye content ≥82 wt %, Sigma-Aldrich) in aqueous solution under solar irradiation. In the presence of the nanosheets, the gradual decrease in the intensity of MB absorbance peak at 664 nm was recorded by using a UV-Visible spectrometer (UV-Vis, PerkinElmer Lambda 35, aperture 20 mm×10 mm). The concentration of the nanosheet samples was set to 0.5 mg/mL in 50 mL of a 1×10⁻⁵ M MB solution. Before irradiation, the suspensions were stirred with the nanosheets for 15-20 min in dark condition to eliminate the role of adsorption desorption-equilibrium between the dyes and the surface of nanosheets during light irradiation. The suspension was illuminated by 100 mW/cm² irradiance power under simulated 1 sun AM 1.5 light, for 0-120 min at 20 min intervals. The optical absorption was measured within the range of 400-800 nm after isolating the CeO₂₋ₓ and heterojunction nanostructures by centrifugation (10000 g, 10 min). The degradation of the MB solutions was assessed by ultraviolet-visible absorbance spectrophotometry (UV-Vis, PerkinElmer Lambda 35 UV-visible spectrometer, aperture 20 mm×10 mm), with quantification being based on the absorption determined by the peak intensity at 664 nm. The high photocatalytic stability of the heterojunctions nanostructure was tested by the use of the same samples for repeating the photodegradation tests.

### Carbon Monoxide (CO) Conversion Test

CO oxidation catalytic activity was evaluated using a fixed-bed quartz micro-reactor (i.d.=6.0 mm). 50 mg of the catalyst sample was placed on a bed of quartz wool in the reactor, and the system purged with N₂ gas for 20 min. The reactant gas, comprising CO (10 sccm) and O₂ (25 sccm) in N₂ (100 sccm), was then introduced at an initial temperature of 30° C. without any catalyst pre-treatment (space velocity of 162 000 mL/(gcat·h)). The temperature was increased incrementally to 150° C. with the step size dependent on the point in the light-off curve. The composition of the exiting gases was evaluated with a Young Lin-6100 gas chromatograph equipped with a thermal conductivity detector (TCD) and a Carboxen-1010 PLOT column.

### Example 1: Synthesis of Metal Coordination Polymers

### 1.1 Synthesis of Ce-CP

The synthesis of Ce-CP tubes was carried out by chronopotentiometry electrodeposition using an electrochemical station (Ezstat Pro, Indiana, USA), with a resolution of 300 μV and 3 nA (in the ±100 μA range) with an undivided three-electrode configuration system. Fluorine-doped tin oxide on glass (FTO; Wuhan Geo Scientific Education Instrument, China; 3.0 cm×1.5 cm; film resistivity ˜16 Ω/sq2), platinum wire (Basi Inc., Indiana, USA, L=23 cm, D=0.5 mm), and Ag/AgCl (Basi Inc., Indiana, USA) were used as the working, counter, and reference electrodes, respectively. The electrolyte was prepared from a mixture of 0.05 M glacial trichloroacetic acid (TCA) and 0.05 M Ce(NO₃)₃.6H₂O. While the pH of the as-prepared aqueous solution was measured to be ˜3, the pH was increased using 1 M NaOH solution to 6 while magnetic stirring at 500 rpm. Prior to electrodeposition, each substrate was cleaned stepwise by ultrasonication in ethanol and acetone for 5 min, followed by activation by immersion (1 cm) in 45% nitric acid for 2 min and drying with compressed nitrogen. The anodic electrodeposition was carried out at room temperature over 50 min by applying the high voltage of 1.2 V vs Ag/AgCl; critically, this is in the water oxidation region. Consequently, the electrodeposition involved oxygen bubbling at the FTO working electrode and hydrogen bubbling at the Pt counter-electrode. The depositions were rinsed by gentle spraying with DI water and dried at room temperature in air. Chronopotentiometry electrodeposition at an applied voltage of 1.4V was used to deposit the Ce-CP tubes. FIG. 49(a) provides a representative schematic of a three-electrode electrochemical cell used for the synthesis of Ce-CP tubes under vigorous oxygen evolution and deposition of free-standing hexagonal tubes of Ce-CP on fluorine-doped tin oxide (FTO) substrate.

### 1.2 Synthesis of Ti-CP

Ti-CP was prepared by injecting an ice-cold solution of TiCl₄ (27.41 μL, 0.25 mmol) into a mixture of DMF (4 mL) and formic acid (7.5 mL) followed by heating at 100° C. for 16 h. The as-synthesized powder was subsequently washed with DMF and acetone via three cycles of centrifugation (5000 g, 20 min) and the obtained Ti-CP powder was dried at 60° C. for 24 h under vacuum.

### 1.3 Synthesis of Zr-CP

In a typical procedure, ZrCl₄ (58 mg, 0.25 mmol) was added to a mixture of dimethylformamide (DMF; 4 mL) and formic acid (7.5 mL) followed by sonication at room temperature for 10 min. The obtained clear solution was then transferred into a Teflon-lined stainless steel vessel and was heated at 100° C. for 16 h. After cooling to room temperature, the resultant white powder was washed three times with DMF (5000 g, 20 min) and then solvent-exchanged with acetone. The final product was dried at 60° C. for 24 h under vacuum to remove the solvents.

### 1.4 Synthesis of MOF-5.

For MOF-5 preparation, Zn(NO₃)₂.4H₂O (3.14 g, 15.8 mmol) was added to a mixture of dimethylformamide (DMF; 100 mL) and terephthalic acid (0.665 g, 4 mmol) followed by stirring at room temperature for 15 min. The obtained clear solution was then transferred into a Teflon-lined stainless steel vessel and was heated at 105° C. for 24 h. After cooling to room temperature, the white precipitate was dispersed in chloroform (100 mL) and stirred for 24 h for solvent exchange. Then, it was dried at 105° C. under vacuum for 24 h.

### Example 2: Synthesis of Nanostructures

### 2.1 Synthesis of CeO₂ Nanosheet

The Ce-CP powder (50 mg) was added to 50 mL of DI water (pH ˜7) and then stirred (100 rpm) for 5 min followed by ultrasonication at room temperature for 10 min. Then, 10 ml NaOH solution (3 M) was added dropwise, resulting in the transformation of the Ce-CP into CeO₂₋ₓ. The resultant nanosheets were collected and washed with DI water. The final product was then air-dried at 100° C. for 24 h. FIG. 49(b) provides a schematic illustration of CeO₂₋ₓ formation through the three-step process including exfoliation of the Ce-CP tubes into Ce-CP nanosheets and subsequently oxidation of Ce-CP nanosheets into holey CeO₂₋ₓ nanosheets.

### 2.2 Large-Scale CeO₂₋ₓ Nanosheet Synthesis

700 mg of Ce-CP was added to 200 mL of DI water at room temperature followed by stirring for 72 h using a magnetic stirrer (100 rpm). Large sheets with a width of up to 0.5 cm were produced in this way. These large-scale sheets were basically formed from stacking of atomic-scale thin nanosheets that were formed in DI water. Longer times usually resulted in the synthesis of wider and thicker sheets. Addition of NaOH (3 M) converted the Ce-CP to CeO₂₋ₓ. Next, the dispersed phase was filtered using a filter paper to separate the CeO₂₋ₓ sheets from the liquid. The resultant sheets were dried at 100° C. for 12 h in an oven. This approach resulted in large-scale production of CeO₂₋ₓ nanosheets.

### 2.3 Synthesis of TiO₂ Nanosheet

TiO₂ nanosheets were prepared by adding 10 mg of Ti-CP powder into 5 mL of DI water followed by stirring (500 rpm) at room temperature for 3 h. Then, 5 mL of NaOH (0.1 M) solution was added to the mixture, and the stirring was continued at room temperature for 2 h. The obtained turbid mixture was washed three times with DI water (10,000 g, 20 min) and the resulting nanosheets were dried at 60° C. for 24 h.

### 2.4 Synthesis of ZrO₂ Nanosheet

ZrO₂ nanosheets were prepared by adding 10 mg of the Zr-CP powder into 5 mL of DI water followed by stirring (500 rpm) at room temperature for 3 h. Then, 5 mL of NaOH (0.1 M) solution was added to the mixture, and the stirring was continued at room temperature for 2 h. The obtained turbid mixture was washed three times with DI water (10,000 g, 20 min) and the resulting nanosheets were dried at 60° C. for 24 h.

2.5 Synthesis of Fe₂O₃/Fe₃O₄—CeO₂₋ₓ (FCO)

The CeO₂₋ₓ nanosheets were first prepared by adding 24 mg of Ce-CP powder into 15 mL of DI water followed by increasing pH to 8 and stirring (100 rpm) at room temperature for 30 min. Then, 5 mL of iron (II) chloride (FeCl₂) solution (0.3 mM) was added to the acidic Ce-CP nanosheet solution (pH=6) followed by addition of 2 mL of NaOH (1 M) under gentle stirring which was continued for 30 min. The resultant turbid mixture was washed with DI water (10000 g, 40 min) and heated at 200° C. for 24 h.

### 2.6 Synthesis of NiO—CeO₂₋ₓ(NCO)

The CeO₂₋ₓ nanosheets were first prepared by adding 24 mg of Ce-CP powder into 15 mL of DI water followed by increasing pH to 8 and stirring (100 rpm) at room temperature for 15 min. Then, 5 mL of nickel (III) nitrate (Ni(NO₃)₂.6H₂O) aqueous solution (0.3 mM) was added to the acidic Ce-CP nanosheet solution (pH=6) followed by addition of 2 mL of NaOH (1M) under gentle stirring which was continued for 30 min. The resultant turbid mixture was washed with DI water (10000 g, 40 min) and heated at 200° C. for 24 h.

### 2.7 Synthesis of ZnO—CeO₂₋ₓ(ZCO)

The CeO₂₋ₓ nanosheets were first prepared by adding 24 mg of Ce-CP powder into 15 mL of DI water followed by increasing pH to 8 and stirring (100 rpm) at room temperature for 15 min. Then, 5 mL of zinc (II) nitrate (Zn(NO₃)₂.6H₂O) aqueous solution (0.3 mM) was added to the acidic Ce-CP nanosheet solution (pH=6) followed by addition of 2 mL of NaOH (1 M) under gentle stirring continued for 30 min. The resultant turbid mixture was washed with DI water (10,000 g, 40 min) and heated at 200° C. for 24 h.

2.8 Synthesis of Different CeO₂₋ₓ Nanostructures from Ce-CP

Tubular nanostructure. The Ce-CP powder (400 mg) was statically aged in NaOH aqueous solution (200 mL, 3M) at room temperature for 30 min. Then, the tubes were washed with water (DI) by three times centrifugation at 5000 g (10 min). The collected tubes were then air-dried at 80° C. for 24 h.

Cubic nanostructure. The Ce-CP powder (100 mg) was added to 100 mL of NaOH solution (10 M) and mixed using a magnetic stirrer (300 rpm, 5 min) at room temperature. Next, the obtained solution was hydrothermally processed at 140° C. for 24 h. The resultant cubes were washed three times by centrifugation at 7000 g (10 min). The final precipitate was then air-dried at 80° C. for 24 h.

Dumbbell-like nanostructure. The Ce-CP powder (100 mg) was added to 100 ml of DI water with an acidic pH of 5 under slow stirring (100 rpm) at room temperature. Then, the solution was calcined at 350° C. (slow rate of 1° C./min) for 2 h. The obtained powders were washed by three cycles of centrifugation (5000 g, 10 min). The final product was then air-dried at 80° C. for 24 h.

Rhombohedral nanostructure. The Ce-CP (10 mg) was dissolved in acetone (4 mL) with stirring (300 rpm) at room temperature for 10 min. The resultant solution was then recrystallized into rhombohedral Ce-CP at room temperature. The obtained nanoparticles were then collected and statically aged in NaOH solution (3 M) for 30 min to transform to CeO₂₋ₓ nanostructure. Then, the final product was washed three times with DI water (3000 g, 10 min) and air-dried at 80° C. for 24 h Flower-like nanostructure. The Ce-CP (40 mg) was dissolved in acetone (2 mL) with stirring (300 rpm) at room temperature for 10 min. The resultant solution was then spread on a glass substrate and recrystallized to form a flower-shaped Ce-CP at room temperature. The obtained Ce-CP nanostructure was then statically aged in NaOH solution (3 M) for 30 min to transform into CeO₂₋ₓ. Finally, the resultant nanoflowers were washed three times with DI water (5000 g, 15 min) and air-dried at 80° C. for 24 h.

Hollow sphere nanostructure. The Ce-CP (40 mg) was dissolved in 4 mL of ethanol under stirring (100 rpm) for 10 min at room temperature. The resultant solution was then recrystallized at low temperature 0° C. for 24 h to form hollow spheres of Ce-CP. The resultant nanostructures were then statically aged in concentrated NaOH solution (3 M) for 30 min and the obtained CeO₂₋ₓ hollow spheres were washed in water collected by three cycles of centrifugation (5000 g, 10 min). The collected hollow spheres were then dried at 80° C. for 24 h in air.

Hollow octahedral nanostructure. The Ce-CP (40 mg) was dissolved in ethanol (4 mL) with magnetic stirring (100 rpm) at room temperature for 10 min. The solution was then allowed to recrystallize at room temperature to form hollow octahedral morphology of Ce-CP. The obtained Ce-CP nanostructure was then aged in aqueous solution of NaOH (3 M) for 30 min to transform into CeO₂₋ₓ. The final CeO₂₋ₓ powder was washed by three cycles of centrifugation (5000 g, 15 min) followed by air-drying at 80° C. for 24 h.

Solid sphere nanostructure. The Ce-CP (40 mg) was dissolved in 40 mL of ethanol under stirring (100 rpm) continued for 10 min at room temperature. The solution was then transferred into a Teflon-lined steel autoclave reactor for the hydrothermal process (140° C., 24 h). The obtained spheres were centrifuged and re-dispersed in water three times (7000 g, 10 min) and the final precipitate was air-dried at 80° C. for 24 h.

2D-3D scaffold nanostructure. The Ce-CP (300 mg) was added to 4 mL of triethanolamine (TEA) and mixed using a magnetic stirrer (100 rpm, 10 min) at room temperature. The mixture was then heated to 450° C. with heating rate of 6° C./min and dwelling time of 3 h. The resultant powder was then cooled and collected for further characterizations.

Solid octahedral nanostructure. The Ce-CP (400 mg) was added to 10 mL of dimethyl sulfoxide (DMSO) at room temperature under gentle stirring continued for 10 min. The solution was then allowed to slowly recrystallize at room temperature to obtain octahedral morphology of a Ce-CP. The resultant Ce-CP nanostructure was subsequently aged in an aqueous solution of NaOH (3 M) for 30 min to transform to CeO₂₋ₓ. The final dispersion was then washed with DI water via three cycles of centrifugation (5000 g, 10 min) followed by air-drying at 80° C. for 24 h.

Honeycomb scaffold nanostructure. The Ce-CP (40 mg) was added to 100 mL of dimethyl sulfoxide (DMSO) under gentle stirring continued for 10 min at room temperature. The solution was kept at low temperature of 0° C. for 2 h to form honeycomb scaffolds at the liquid-air interface. The resultant scaffolds were then collected by touch-printing on a clean glass substrate. The obtained honeycomb scaffold was then heated to 350° C. and maintained for 2 h to transform to CeO₂₋ₓ nanostructure.

### General Procedure for the Synthesis of Ce-CP Derived CeO₂₋ₓ Nanostructures Through Disassembly/Reassembly in a Polar Solvent

In order to synthesise the 3D CeO₂₋ₓ morphologies, different concentrations of Ce-CP precursors in the range of 4 M to 120 M (the full range of concentrations, temperatures, and resultant morphologies are given in Table A below) were added to pure ethyl alcohol (96.0-97.2%) as solvent, followed by stirring at room temperature for 5 min. The resultant yellow solutions, which are indicative of the Ce⁴⁺ ion, were evaporated at different rates by adjusting the temperature in the range of 0° C. to +25° C., which resulted in recrystallisation of the Ce-CP in various morphologies. Temperatures less than 25° C. (room temperature) were achieved with the use of a freezer with an inserted temperature probe.

The transformation of Ce-CP into CeO₂₋ₓ was affected by immersing the Ce-CP morphologies in 6 M NaOH aqueous solution and oxidising for 30 min followed by rinsing by spraying with DI water and complete drying by heating in an oven at 200° C.

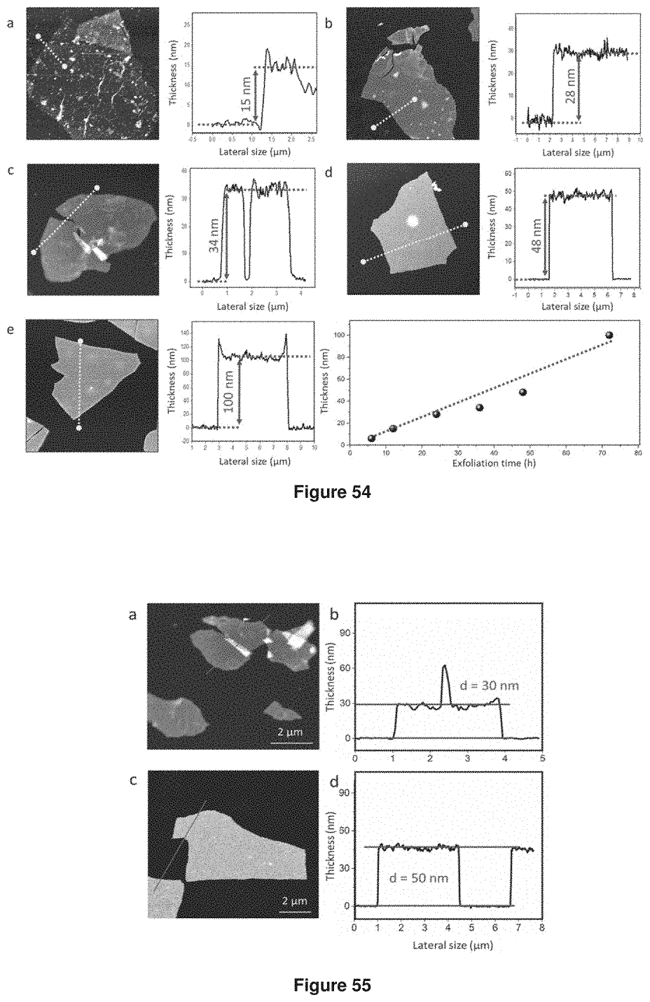

The synthesis of the 2D CeO₂₋ₓ morphologies was done in an identical manner with the following exceptions. The evaporation temperatures were in the range −10° C. to 0° C.; the corresponding vapour pressures are given in Table A. For thickness measurements as a function of drying time, the Ce-CP nanosheets were deposited on glass substrates using the touch-print technique. Nanosheets of varying thicknesses were obtained by controlling the evaporation time for 6 h to 72 h; the resultant data are given in FIG. 54. Further, nanosheets were obtained at constant temperature of −10° C. but different Ce-CP concentrations, the AFM results of which are shown in FIG. 55.

TABLE A
Effects of concentration, temperature, and vapour pressure
(ideal gas) on morphological variations of Ce—CP.

| Concen- | Temper- | Vapour | |
|---|---|---|---|
| tration | ature | Pressure | |
| (M) | (° C.) | (kPa) | Nanostructure |
| 4-30 nm | −10 | 0.744 | thin nanosheet |
| 8-48 nm | | | |
| 4 | 0 | 1.568 | thick nanosheet |
| 16 | | | hollow sphere |
| 4 | +25 | 7.830 | hollow sphere |
| 8 | | | hollow pseudoctahedron |
| 40 | | | hollow elongated octahedron |
| 120 | | | solid leaf |

2.9 Synthesis of Different ZnO Nanostructures from MOF-5

Spherical nanostructure. The as-synthesized MOF-5 (100 mg) was dispersed into a vial containing tetrapropylammonium hydroxide (TPAOH, 2 mL, 40 wt %) and stirred at room temperature for 5 min. Then, the dispersion was cooled down to 0° C. and statically maintained for 72 h. After the growth nanocrystal, the precipitate was washed with EtOH (40 mL) three times and air-dried at room temperature.

Needle-like nanostructure. The MOF-5 (100 mg) was dispersed in tetrapropylammonium hydroxide (TPAOH, 2 mL, 40 wt %) and transferred into a Teflon-lined autoclave for the hydrothermal process (140° C., 24 h). The resultant precipitate was washed with EtOH (40 mL) three times and air-dried at room temperature.

Rod-like structure. The MOF-5 (100 mg) was added into a vial containing H₂O (40 mL) and stirred (500 rpm) at 40° C. for 12 h. The dispersion was then washed with EtOH (40 mL) three times and air-dried at room temperature.

Bundle-like nanostructure. The MOF-5 (100 mg) was added into a vial containing acetone (39 mL) and KOH solution (1 mL, 10 M) and stirred slowly at 40° C. for 12 h. The resultant precipitate was washed in EtOH (40 mL) by three cycles of centrifugation (4000 g, 20 min) and the final product was air-dried at room temperature.

Wafer-like nanostructure. The MOF-5 (0.1 g) was dispersed into a vial containing tetrabutylammonium hydroxide (TBAOH, 2 mL, 40 wt %) and stirred at room temperature for 5 min and maintained under the static condition for 12 h. The obtained precipitate was then washed with EtOH (40 mL) via three cycles of centrifugation (4500 g, 15 min) followed by air-drying at room temperature.

Fibre nanostructure. The MOF-5 (0.1 g) was added into a vial containing ethanol (39 mL) and H₂O (1 mL) and stirred at 40° C. for 12 h. Then the resultant dispersion was centrifuged and re-dispersed in EtOH (40 mL) three times followed by air-drying at room temperature.

### Example 3: Synthesis and Characterisation of Ce-CP

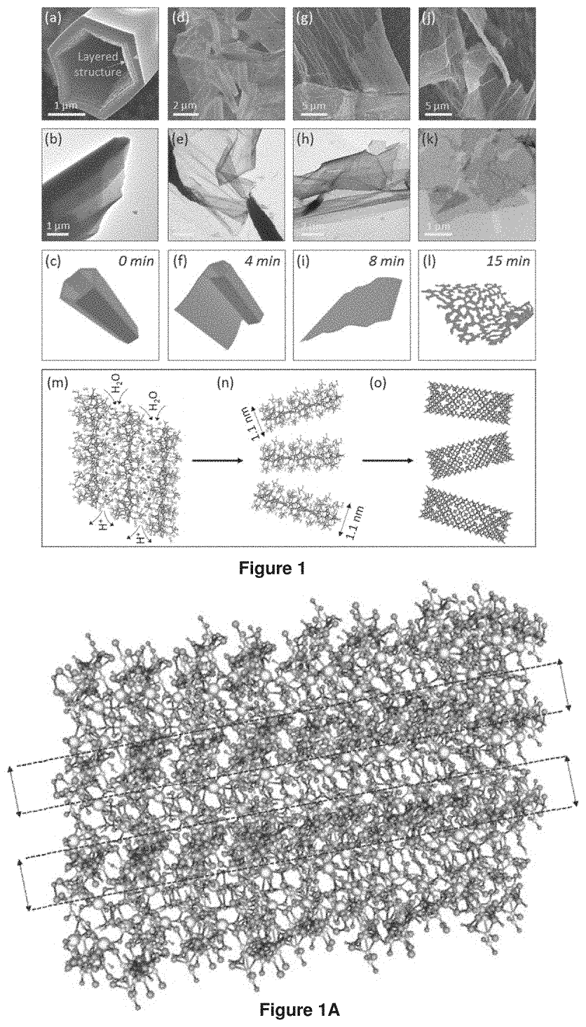

Electrodeposition of the Ce-CP was performed using modified anodic electrochemical deposition (chronoamperometry techniques; referred to as MACE), in which the current varies as a function of deposition time, while a constant potential is applied. FIG. 1a shows scanning electron microscopy (SEM) image of a free-standing Ce-CP hexagonal tube with bulk-layered structure. Additionally, transmission electron microscopy (TEM) image and the corresponding schematic are shown in FIGS. 1b and c, respectively.

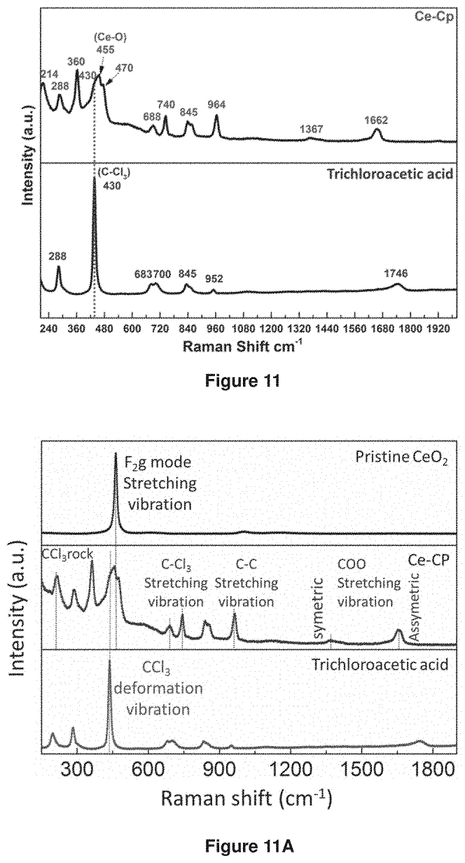

The stratified Ce-CP tube can be readily exfoliated, upon ultrasonication in deionized (DI) at room temperature. FIGS. 1d and e shows ex-situ SEM and TEM images of the Ce-CP partly exfoliated after 4 min ultrasonication. The corresponding schematic is shown in FIG. 1f. Longer sonication treatment (8 min) led to the complete Ce-CP exfoliation, as illustrated by SEM and TEM images in FIGS. 1g and h, respectively. The total exfoliation progress as a function of sonication time is schematically demonstrated in FIG. 1c-i. The final step involves increasing the pH of the solution to pH=8, during ultrasonication, leading to the transformation of the Ce-CP nanosheets into defective CeO₂₋ₓ nanosheets. It is significant to note that, during this transformation, high densities of nanoholes across the ultrathin sheets are formed as shown in FIGS. 1j and k. This is attributed to the rapid removal of the organic bidentate trichloroacetate (TCA) linkers, owing to high field strength of Ce(IV) over a wide pH range and thus a corresponding strong affinity for CeO₂₋ₓ formation. The schematic of the holey structure of the CeO₂₋ₓ nanosheet is also shown in FIG. 11, and the schematic of the modified anodic electrochemical deposition technique and subsequent exfoliation and organic linker removal is shown in FIG. 49.

Owing to the absence of reference data consistent with the X-ray diffraction (XRD) pattern obtained for the Ce-CP, the corresponding crystal structure was investigated by comparative ab-initio molecular dynamics simulations and XRD and neutron diffraction patterns, as provided in FIGS. 8-19 and Tables 1-2. The data describing the crystallography of the Ce-CP, which has been determined to be Ce(TCA)₂(OH)₂.2H₂O, was indexed to be triclinic system, space group P1, with a=1.31 nm, b=1.32 nm, c=1.10 nm, α=81.20°, β=93.21°, γ=112.93°.

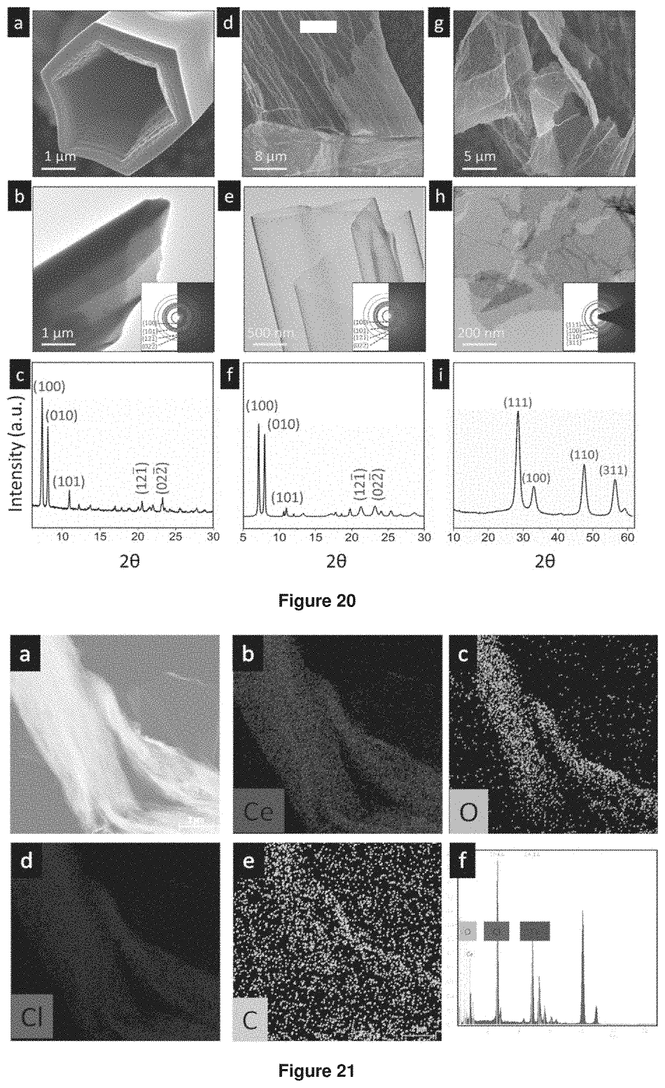

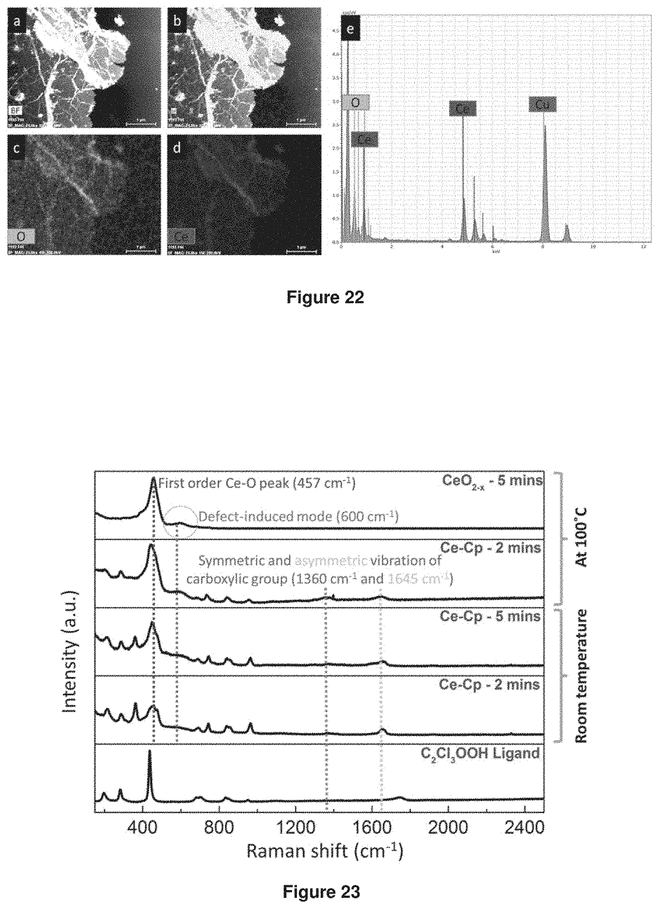

The crystal structure of the stratified Ce-CP is illustrated in FIG. 1m, where the interlayer spaces are mutually held together by intercalated protons and the terminating chlorine ions of the TCA ligands. The application of ultrasonication on the Ce-CP tubes enhances the exfoliation through the vibration's breakage of the nanosheet and resultant facilitated water molecule penetration (FIG. 1n). Further, the c-axis lattice parameter of the Ce-CP crystal structure was measured to be 1.1 n, which represents the thinnest possible Ce-CP nanosheet of a Ce-CP monolayer. Increasing the pH of the solution, leads to dissolution of the TCA from the two surfaces (FIG. 1n) of the M-OH substructure. This is followed by conversion of a highly reactive interior M-OH(Ce(OH)₂²⁺) substructure to the more stable Ce(OH)₄ followed by the rapid formation of stable CeO₂-x (FIG. 1o) without any morphological changes. The structural evolution during the Ce-CP transformation into CeO₂₋ₓ is studied using XRD and SAED analysis (FIG. 20). In order to confirm the removal of the TCA, energy dispersive spectroscopy (EDS) elemental mapping was carried out for both Ce-CP and CeO₂₋ₓ nanosheets, as shown in FIGS. 21 and 22, respectively. Furthermore, the rapid evolution of Ce-CP into CeO₂₋ₓ is studied by in situ laser Raman microspectroscopy of nanosheets subjected to an alternative removal method (FIG. 23).

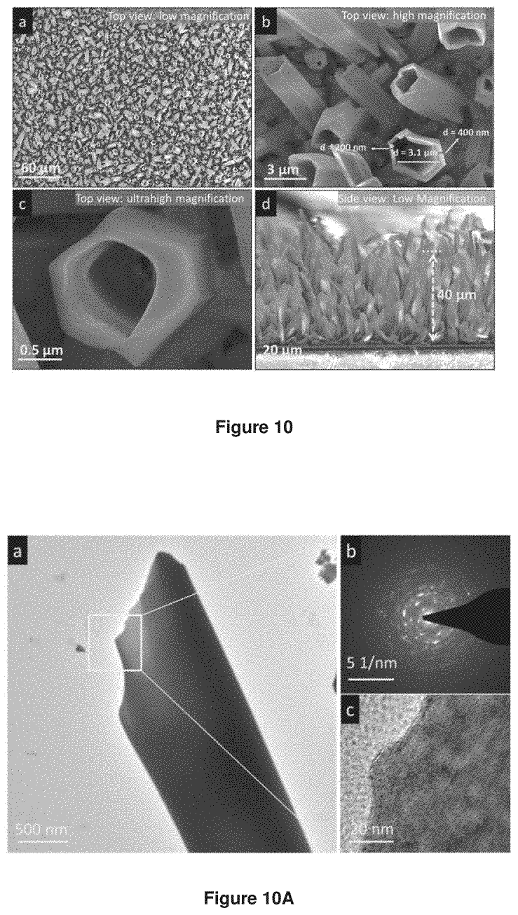

FIG. 6 shows current-deposition time plot, where the current density increases rapidly for the initial stages of the deposition. The high current density is attributed to the oxygen evolution reaction at the working electrode (FTO substrate). However, the current density drops after ˜100 s of applying a potential followed by a gradual decrease after ˜160 s. The variations in current density were studied by analysis nucleation/growth mechanism using SEM imaging, as a function of deposition times (inset of FIG. 6). The image obtained at the peak current density (FIG. 6b) revealed small nuclei of the Ce-CP. The low-conductivity of the Ce-CP polymer, compared to the FTO substrate is likely to result in a drop in the current density. The continued growth of the Ce-CP polymer led to a decrease in the exposed FTO surface and thus, reduction of the current density. Interestingly, FIG. 10c shows that nuclei are grown vertically against the substrate while forming a hexagonal rod after 150 s of deposition. Increase in the deposition time (FIG. 6d) resulted in the formation of a hole at the centre of the hexagonal rod and finally, the transition of the hexagonal rod to the hexagonal tube (FIG. 6f). This transition can be attributed to the effect of the application of high current density, which resulted in the travel of generated oxygen bubbles perpendicular to the substrate. Therefore, the evolution of morphology moves towards minimization of the Ce-CP contact surface with the FTO substrate, owing to enhanced accessibility of water at the FTO substrate leading to subsequent oxygen evolution reactions.

Applying high current density in the oxygen evolution region resulted in a high production rate of oxygen bubbles at the region adjacent to the FTO substrate. The high O₂ concentration environment results in an oxidation of Ce(III) species to Ce(IV), which is shown as the step (1) in FIG. 7. From the other side, according to water splitting reaction, the evolution of one mole oxygen is followed by the formation of 4 moles of protons that lead to a rapid drop in the local pH and result in the formation of a highly acidic atmosphere. All these reactions occur above the water stability range labelled blue in FIG. 7.

The stability regions of Ce(IV) species in aqueous solution exist due to the high field strength (affinity to hybridization) of Ce(IV) species. Therefore, the oxidation of Ce(III) species to Ce(IV), even under acidic pH, results in the formation of Ce(IV) hydroxide but with unsaturated coordination bonds. This is also shown in the Pourbaix diagram (FIG. 7), where the formation of Ce(IV) hydroxide with low coordination number occurs followed by the rapid proton generation (step(2)). In the final step, the presence of TCA molecules with unstable Ce(IV) species leads to coordination bonding between the Ce(IV) hydroxide and TCA linker, forming a monolayer structure. The existence of high concentration of protons, owing to the acidic pH, allows the protons to intercalate at the interlayer spaces of Ce(IV) and TCA coordinated monolayer structure and establish Van der Waals interactions between the layers resulting in the formation of (Ce(OH)₂(TCA)₂.2H₂O).

The possible chemical reactions towards the formation of Ce—CP are as follows:

(1) Deprotonation of TCA in water followed by dropping in pH value from 6.5 to <2.3:

CCl₃COOH+H₂O→H₃O⁺+CCl₃COO⁻ (Eq. 1)

(2) Dissociation of cerium nitrate salt in the solution resulting in the release of free Ce³⁺ and nitrate anions:

Ce(NO₃)₃.6H₂O→Ce³⁺+3NO₃⁻+6H₂O (Eq. 2)

(3) At pH=6, the oxidation voltage for cerium was found to be 0.55 V vs. Ag/AgCl, while the onset of water oxidation is laid at 0.8 V. Applying constant potential of 1.2 V vs. Ag/AgCl caused a rapid generation of oxygen at the anode (FTO) surface (Equations 3-5):

2H₂O→4H⁺+O₂+4e⁻ (Eq. 3)

4OH⁻→4H⁺+2O₂+4e⁻ (Eq. 4)

HO₂⁻+OH⁻→H₂O+O₂+2e⁻ (Eq. 5)

The high production rate of oxygen molecules on the FTO substrate results in oxidation of Ce(III) species to Ce(IV). However, during water oxidation, the evolution of one-mole oxygen is followed by the formation of 4 mole protons that in turn results in a rapid drop in local pH and an increased concentration of protons. At this condition, Ce(IV) hydroxide species are in soluble form. Additionally, owing to low pKa value of the TCA, deprotonated TCA acted as secondary building units (SBUs), bridging Ce(IV) hydroxide species together resulting in the formation of a novel polycrystalline Ce-CP. The corresponding equation is given below:

Ce⁴⁺+2OH⁻+2TCA+2H₂O═Ce(OH)₂(TCA)₂.2H₂O (Eq. 6)

The stratified Ce-CP tube can be readily exfoliated, upon ultrasonication in deionized (DI) at room temperature. FIG. 1d and 1e shows ex-situ SEM and TEM images of the Ce-CP partly exfoliated after 4 min ultrasonication. The corresponding schematic is shown in FIG. 1f. Longer sonication treatment (8 min) led to the complete Ce-CP exfoliation, as illustrated by SEM and TEM images in FIGS. 1g and h, respectively. The total exfoliation progress as a function of sonication time is schematically demonstrated in FIG. 1c-i. The final step involves increasing the pH of the solution to pH=8, during ultrasonication, leading to the transformation of the Ce-CP nanosheets into defective CeO₂₋ₓ nanosheets. It is significant to note that, during this transformation, high densities of nanoholes across the ultrathin sheets are formed as shown in FIGS. 1j and k. This is attributed to the rapid removal of the organic bidentate trichloroacetate (TCA) linkers, owing to high field strength of Ce(IV) over a wide pH range and thus a corresponding strong affinity for CeO₂₋ₓ formation. The schematic of the holey structure of the CeO_{2X} nanosheet is also shown in FIG. 11.

The Raman spectra of the Ce-CP (FIGS. 11 and 11A) were analysed comprehensively and indexed according to the vibrational modes of pure TCA and CeO₂. The data indicates that some of the peaks observed in Ce-CP spectra are also present in TCA spectra, indicating the existence of TCA molecule in the Ce-CP. The peaks centred at 288 and 430 cm⁻¹ are attributed to the asymmetric and symmetric bending vibrations of the C—Cl bond, respectively. Additionally, the peak at 688 cm⁻¹ belongs to symmetric stretching vibration mode of C—Cl bond, while peaks at 845 and 744 cm⁻¹ are due to asymmetric stretching vibration mode of the same bond. The peak positioned at 952 cm⁻¹ corresponds to the symmetric vibration mode of the carbon-carbon bond (C—C). Further comparison of the two spectra shows that Raman shifts occurred in some of the peaks (952 cm⁻¹ to 962 cm⁻¹, 700 cm⁻¹ to 740 cm⁻¹, and 683 cm⁻¹ to 688 cm⁻¹), which are attributed to the alteration in vibrational modes of the bonds in TCA structure due to interaction with cerium ions. Additionally, in TCA, there is a peak at 1746 cm-1 corresponding to the vibrational mode of the free carboxylic group (COO), which is split into two peaks at 1367 (symmetric stretching vibration) and 1662 cm⁻¹ (asymmetric stretching vibration) in Ce-CP spectra. The splitting seems likely due to the interactions between the COO group and Ce that results in the formation of Ce—O bond, the peak of which appears at 455 cm⁻¹. The peaks at 214 cm⁻¹ and 360 cm⁻¹ are correlated to in- and out-of-phase vibration modes of the Ce-CP structure. There are two dominant peaks positioned at 455 and 470 cm⁻¹ in Ce-CP spectra. The former is ascribed to the symmetric stretching vibration of cerium and its coordinated oxygen, while the latter originates from the vibration mode of cerium bonded with chlorine and oxygen.

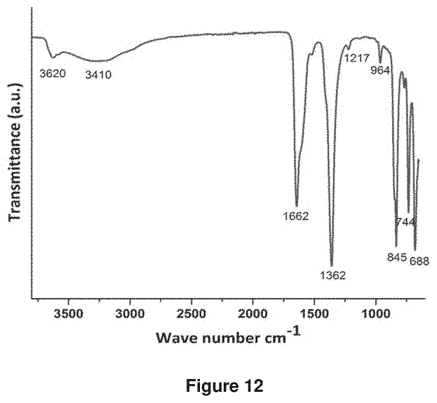

In FTIR spectrum of Ce-CP tubes (FIG. 12), the bands centred at 3620 and 3410 cm⁻¹ show stretching vibration of hydroxyl groups revealing the presence of water and OH group in the Ce-CP. The peaks at 1660 cm⁻¹ and 1360 cm⁻¹ are attributed to the asymmetric and symmetric stretching mode of a carboxylic group that is bonded to cerium cations. Also, the peaks at 1040 cm⁻¹ and 966 cm⁻¹ are due to the bending vibration of the carboxylic group and symmetric vibration mode of the carbon-carbon bond (C—C), respectively. Similar to Raman spectra, the peaks at 688 cm⁻¹, 744 cm-1, and 845 cm⁻¹ are attributed to C—Cl vibration modes.

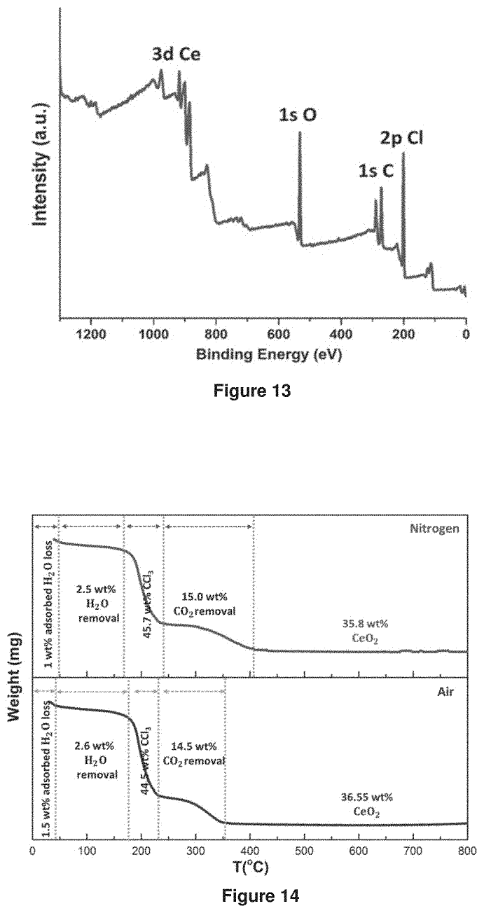

XPS data of Ce-CP tubes (FIG. 13) showed the peaks corresponding to 3d, Is, Is, and 2p orbitals of cerium, oxygen, carbon, and chlorine elements can be detected at binding energies ranging from 880-920, 529-535, 284-292, and 198-202 eV, respectively. For the cerium, there are two oxidation states of Ce³⁺ and Ce⁴⁺ representing a spin-orbit combination of electrons in the d-orbital (3d_{5/2} and 3d_{3/2}). The corresponding binding energies of Ce⁴⁺ and Ce³⁺ in 3d_{5/2} configuration are located at 883, 889, and 899 and 881, and 886 eV, respectively (Table 1). The peak positioned at 530 eV corresponds to hydroxyl bonded to Ce⁴⁺. The peak of organic oxygen in TCA can be observed at 532 eV. At the binding energy of 534 eV, there is a small broad peak representing structural H₂O. All the peak positions are provided in Table 1.

TABLE 1
Binding energies of different chemical
bonds in Ce—CP and TCA.

| | Chemical | Peak Position |
|---|---|---|
| Element | State | Binding Energy (eV) |
| Ce | Ce⁴⁺—O | 883, 889, 899 |
| | Ce³⁺—O | 881, 886 |
| O | OH—Ce⁴⁺ | 530 |
| | O in TCA | 532 |
| | O (H₂O) | 534 |
| Cl | Cl in TCA | 200, 202 |
| | Cl—Ce³⁺ | 198 |
| C | C—TCA | 289 |

The quantitative analysis of the elements in the Ce-CP structure was carried out by deconvolution of the peaks using Gaussian fitting, the results of which are given in Table 2. From the analysis, the stoichiometry of the Ce-CP is identified, based on atomic percentages to be Ce(OH)_{1.8}(TCA)_{2.0}(H₂O)_{1.0}. Further, the XPS results were used for TGA analysis confirming the molar ratio of the Ce-CP structure from wt %, which is elaborated in the following section.

The quantitative analysis of the elements in the Ce-CP structure was carried out by deconvolution of the peaks using Gaussian fitting, the results of which are given in Table 2. From the analysis, the stoichiometry of the Ce-CP is identified, based on atomic percentages to be Ce(OH)_{1.8}(TCA)_{2.0}(H₂O)_{1.0}. Further, the XPS results were used for TGA analysis confirming the molar ratio of the Ce-CP structure from wt %, which is elaborated in the following section.

TABLE 2
Contribution of different elements in Ce—CP with the
stoichiometry based on atomic and weight percentages.

| | | | | Calculated |
|---|---|---|---|---|
| | XPS | General | | XPS |
| | Atomic | Molecular | MW/ | Weight |
| | Contribution | Weight | Avogadro’s | Contribution |
| Element | (at %) | (g/mol) | Number | (wt %) |
| Ce | 5.1 | 140 | 714 | 23.0 |
| | 1.8 | | 256 | 8.2 |
| O | 9.2 | 16 | 147 | 4.7 |
| | 20.9 | | 334 | 10.7 |
| | 4.2 | | 67 | 2.1 |
| Cl | 30.4 | 35 | 1066 | 34.2 |
| | 6.4 | | 224 | 7.2 |
| C | 21.9 | 14 | 307 | 9.9 |
| Total | 100 | — | 3115 | 100 |

TGA analysis of Ce-CP (FIG. 14) showed similar patterns under both nitrogen and air atmospheres. There are four steps during each of which adsorbed water, structural water, carbon chloride, and CO₂ is removed from the Ce-CP, respectively. The results show that 35.8 wt % of the total Ce-CP is converted to CeO₂ in both conditions. The weight percent of each organic component and the resultant product are given in Table 3, with associated XPS analysis data for comparison. The Ce-CP samples after TGA test were examined by XRD and Raman spectroscopy (not shown here), revealing that heat treatment in both gases results in the formation of CeO₂ cubic fluorite structure.

TABLE 3
TGA analysis of Ce—CP in air and nitrogen
and the associated XPS data.

| | TGA (N₂) | | XPS | |
|---|---|---|---|---|
| | Removal | Weight | Calculated | |
| | Temperature | Loss | based | Difference |
| Elements | (° C.) | (wt %) | on XPS data | (%) |
| CeO₂ | N/A | 35.8 | 35.9 | 0.2 |
| H₂O | 160 | 2.5 | 2.1 | 19.0 |
| CCl₃ in | 230 | 45.7 | 46.3 | −1.3 |
| TCA | | | | |
| CO₂ in | 350 | 15.0 | 15.7 | 4.6 |
| TCA | | | | |

### Characterization Data of Ce-CP Crystal Structure

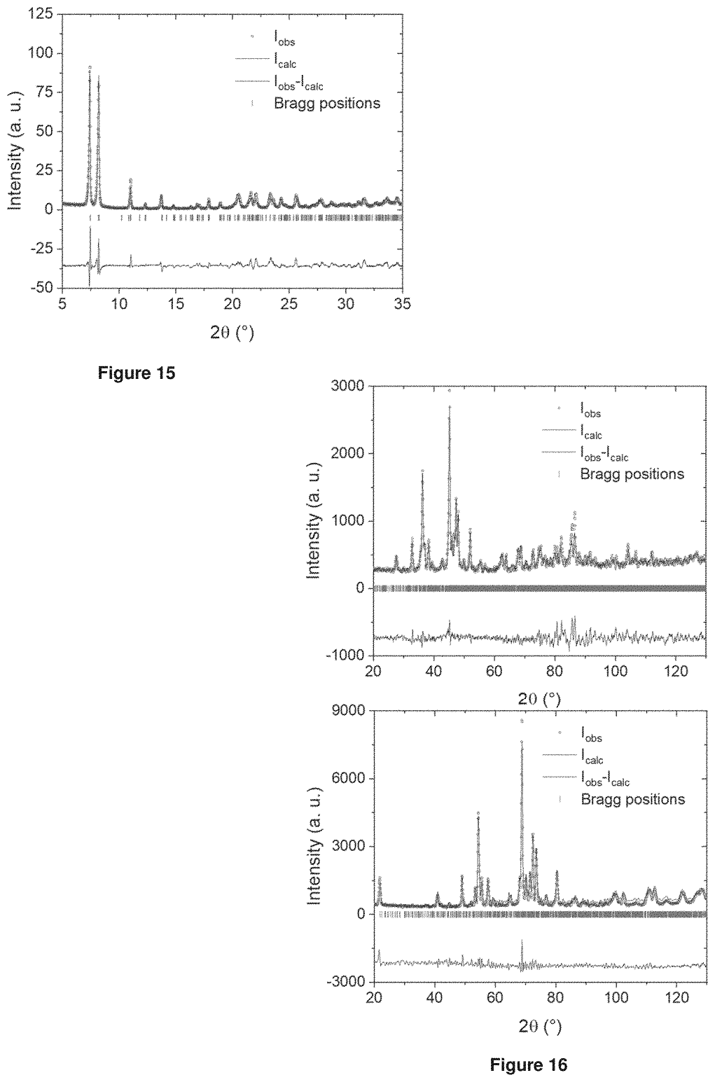

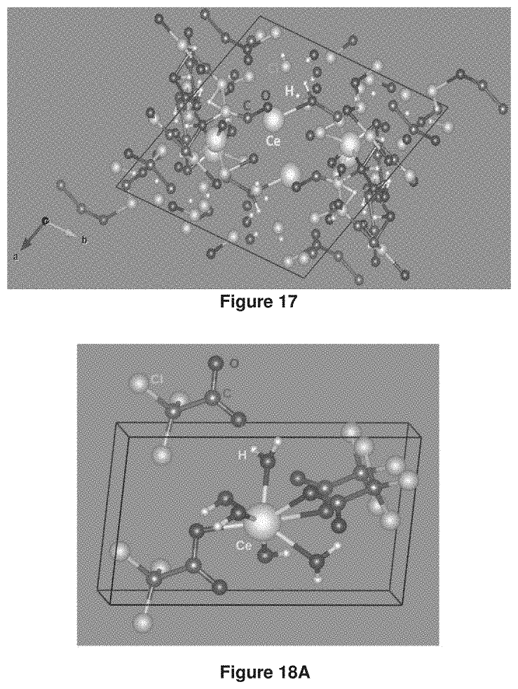

To identify the Ce-CP structure, which does not match with the existing structures in the crystallographic database (The Cambridge Crystallographic Data Centre (CCDC)), attempts were made to produce Ce-CP single crystals through vapour and layer diffusion methods. However, all attempts resulted in the formation of polycrystalline Ce-CP. Therefore, based on the obtained chemical composition of (Ce(OH)₂(TCA)₂.2H₂O), and achieved X-ray (1) and neutron (2) diffraction patterns, a Rietveld refinement (using program package FullProf) was performed, combining all three datasets in order to increase the level of information. The lattice parameters, atomic positions and zero shift parameters were refined. A good fit was obtained for both X-ray and neutron diffraction patterns of Ce-CP (FIGS. 15 and 16). However, the interpretation of the obtained structure from a physical point of view was challenging due to the uncertainty in the location of lighter elements especially H (FIG. 17), while the positions of Ce atoms as well as the lattice parameters were found to be very close to that of experimental data.

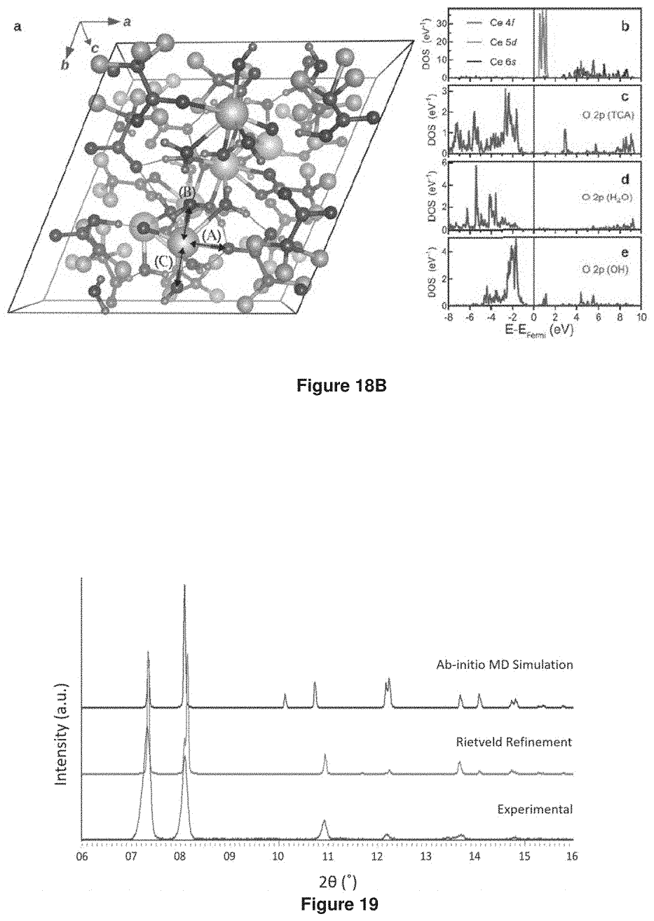

In order to obtain a physically meaningful structure, the refined lattice by the Rietveld method was used as a guideline (particularly the lattice parameters and Ce position) for density functional theory and subsequent ab initio molecular dynamics calculations. To draw an approximate picture of the coordination environment around cerium atom in the Ce-CP structure, a small stoichiometric supercell comprising of Ce(OH)₂(TCA)₂.2H₂O with a sixth of the volume of the refined experimental structure was first used (FIG. 17). Many coordination possibilities were exhaustively compared, such as Ce being coordinated with TCA's Cl and O atoms. The relaxed structure of the most stable coordination is shown in FIGS. 18A and 18B. It was found that Ce was coordinated by seven O atoms; two from the OH group, two from each of the water molecules, and three from the two TCA molecules. The geometry obtained in FIG. 17 was then used to fill the six distinct lattice points of the experimentally refined P1 structure. To find the ground state configuration of a larger supercell, a quenching ab initio molecular dynamics simulation was run based on a micro-canonical ensemble with a target temperature of 20 K with steps of 0.1 fs for 10 ps. The molecular dynamics simulation was effective in finding a reasonable initial structure for geometry optimization. Full geometry optimization was then carried out on the equilibrated structure, with convergence criteria for the energy and forces of 10⁻⁶ eV and 10⁻² eV/A, respectively. The final geometry optimization included the Van der Waals correction (vdw-DFT) based on Michaelides's approach. To analyse the coordination environment of the Ce atoms in the large supercell, the crystal orbital overlap population (COOP) was calculated using LOBSTER code. The bonds connected to Ce atoms were identified by counting all pairs with positive integrated COOP with Ce at one end. Positive integrated COOP values demonstrate that bonding orbitals between Ce and ligands were occupied. It was found that all such bonds were formed between Ce and O. Each Ce atom was found to have bonds with eight neighbouring 0 atoms at an average bond length of 2.61 Å. Six of the Ce—O bonds were found to be rather weak judged by large bond lengths approaching ˜3 Å and meagre integrated COOP values at an average of 0.05. Some of these bonds may break at room temperature due to thermal fluctuations. As a result, the average coordination number of Ce atom at room temperature is predicted to be between seven and eight, matching exactly the XPS and TGA results shown in FIGS. 13 and 14. FIG. 19 compares the low angle peaks of the X-ray diffraction pattern of experimental Ce-CP, Rietveld refined structure and ab initio molecular dynamics simulated structure.

The final optimised structure which is shown in FIG. 18B(a) reproduces the main diffraction peaks at low angles centered at 7.34982° and 813390° with reasonable accuracy. It should be noted that the low resolution of the XRD measurement limits the use of the experimental diffraction pattern for evaluating the DFT optimized structures. Consequently, comparison with the measured pattern can only evaluate the position of larger atoms, whereas finer details such as the H-bond network and the position of hydrogens can barely be assessed based on this comparison.

Furthermore, the Ce-O_{(TCA)} bond was found to be ˜2.56 Å which was longer than both Ce-O_{(H2O)} bond at ˜2.60 Å and Ce-O_{(OH)} bond at 1.96 Å. The longer Ce-O_{(TCA)} bond length reinforces the notion of the fragility of this bond. The empty Ce 4f, 5d and 6s states, in FIG. 18B(b), point to a 4+ oxidation state for Ce ions. Moreover, the lack of any overlap between Ce states and coordinating 0 states (FIG. 18B(b)-(e)) indicates lack of any strong covalent bonding to Ce.

Overall, based on the results described herein, the XRD pattern of the Ce-CP powder was indexed to triclinic Ce(OH)₂(C₂O₂Cl₃)₂.2H₂O, space group P1̅, a=1.29 nm, b=1.31 nm, c=0.81 nm, α=88°, β=92°, γ=112° (FIGS. 18B and 19).

### Photoluminescence Spectra of Ce-CP

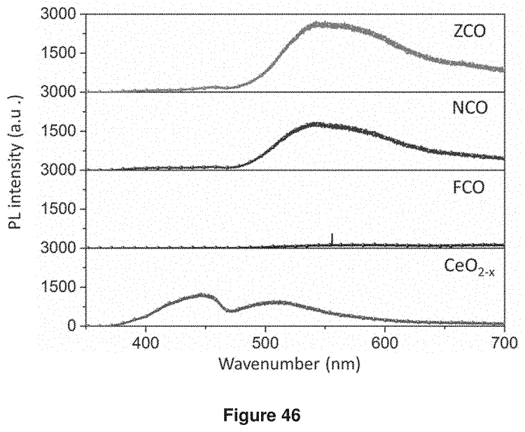

The room temperature photoluminescence (PL) emission of the CeO₂₋ₓ and the heterojunction structures are shown in FIG. 46. The PL spectra for CeO₂₋ₓ nanosheet show two small and broad emissions with a wavelength of 426 nm (blue emissions) and 510 (green emissions). The former is attributed to the F⁺⁺→4f₁ transition, as the F⁺⁺ state is just below the 4f₀ band acting as an electron trap and 4f₁ state acts as a hole trap. However, the latter, originates from the presence of Ce³⁺, as a hole trap state, and oxygen vacancy, as an electron trapping state. The radiative recombination of these two traps leads to the excitation at the wavelength of 510 nm. The low PL intensity of these two emissions for CeO₂₋ₓ nanosheet, compared to the reported CeO₂ nanostructures, again confirms the short diffusion pathways for charge carriers and hence reduction of radiative recombination.

### Example 4: Synthesis and Characterisation of Ti-CP and Zr-CP

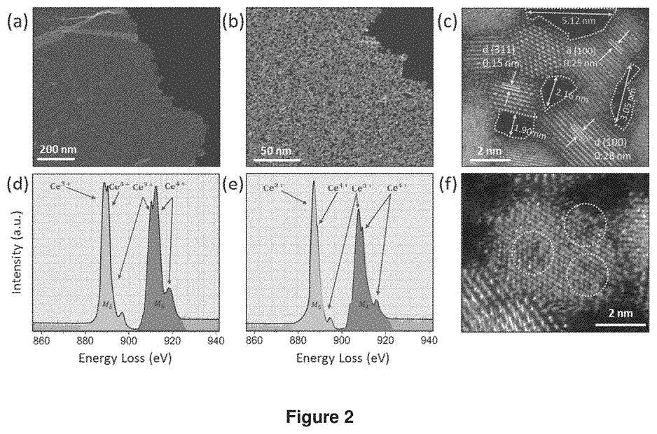

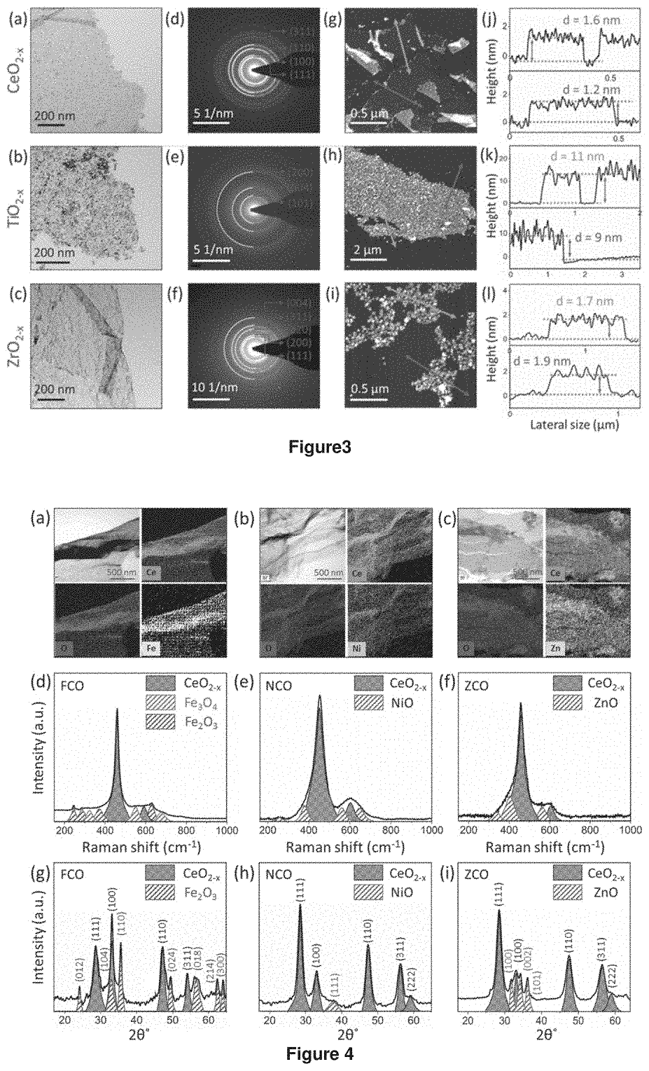

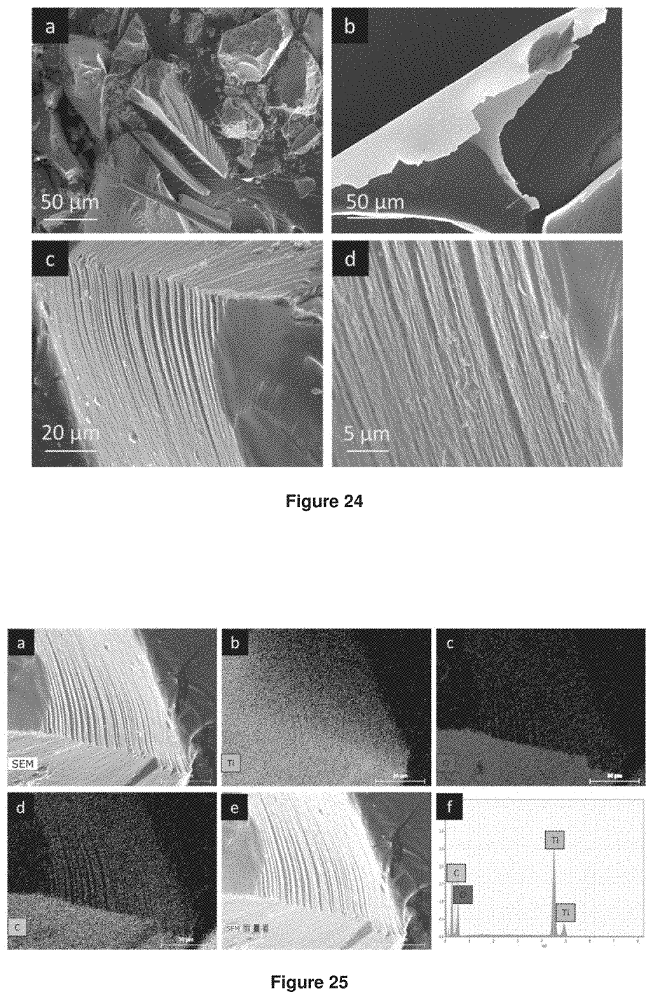

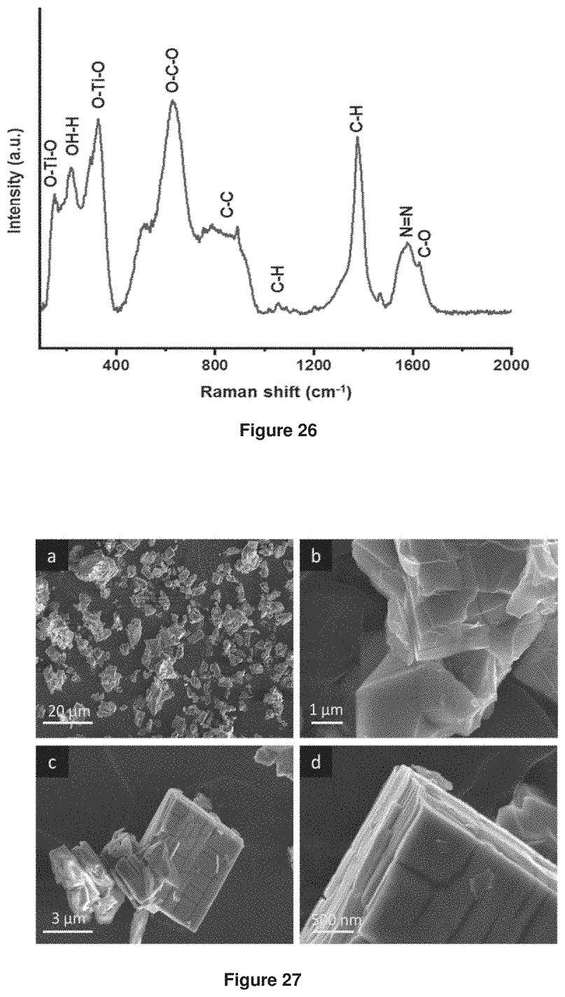

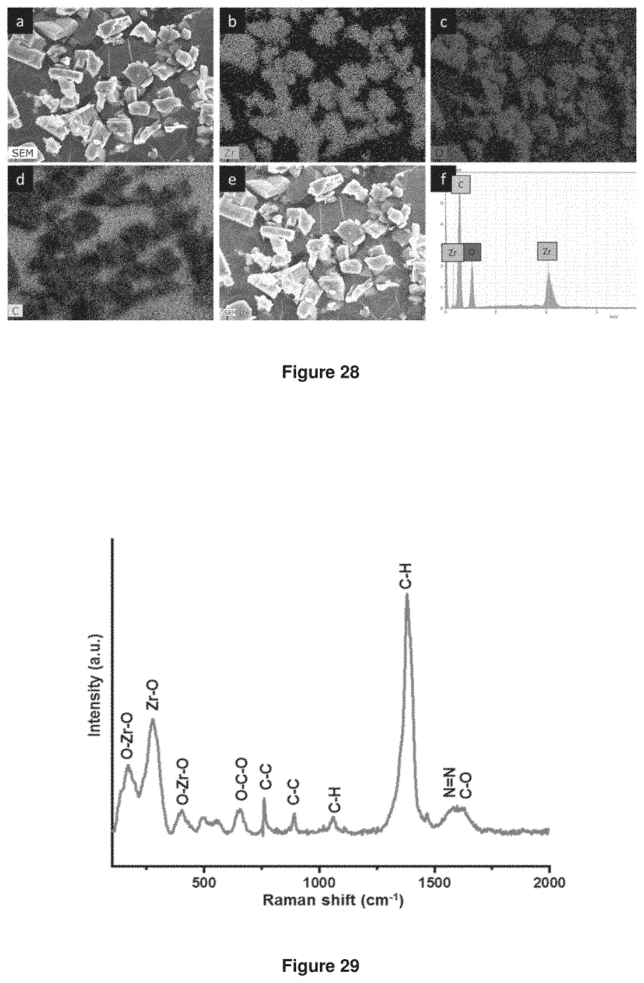

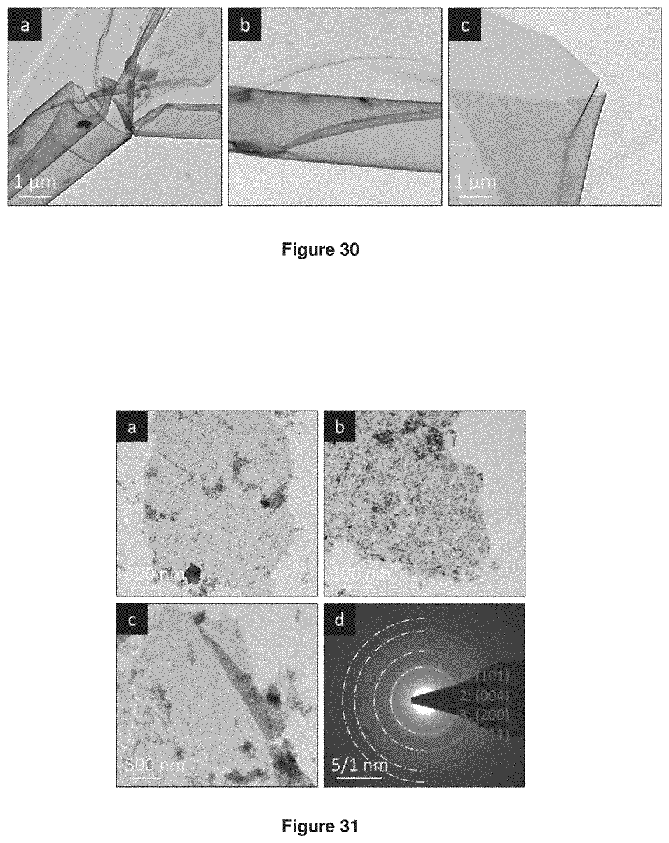

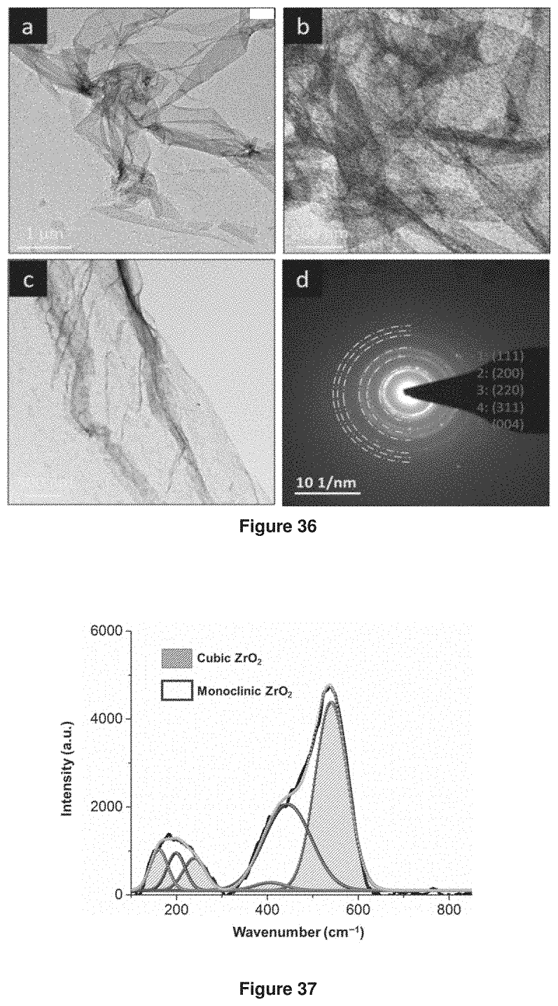

The flexibility of the disclosed fabrication method is confirmed by the syntheses of a layered titanium-based CP (Ti-CP) and a zirconium-based CP (Zr-CP). Details of the morphological and structural characterization of these bulk layered MCPs are given in FIG. 24-29. Similar to Ce-CP, the Ti-CP and Zr-CP were exfoliated rapidly in basic aqueous solutions into nanosheets, as illustrated by TEM and EDS analyses (FIG. 30-39). The morphological analyses of CeO₂₋ₓ, TiO₂₋ₓ, and ZrO₂₋ₓ nanosheets are shown in FIG. 3a-c, respectively, where TEM images reveal the holey nanostructures of the MCP-derived metal oxides. Also, FIG. 3d-f show SAED patterns of the randomly-oriented polycrystalline nanosheets indexed to CeO₂, TiO₂, and ZrO₂, respectively. Considering the ultrathin nature of the holey nanosheets, surface chemical analysis effectively provides bulk analysis since the penetration depth of XPS is ˜3 nm. As an example, quantitative analysis of CeO₂₋ₓ(FIG. 40) was carried out by deconvolution of Ce 3d orbital of XPS spectra revealing significant Ce³⁺ concentrations, which generally are associated with corresponding oxygen vacancy concentrations ([V_{O}^{••}]) through charge compensation. These results are in agreement with the EELS data shown in FIGS. 2d and e.

In order to measure the thicknesses of the holey metal oxide nanosheets, atomic force microscopy (AFM) imaging was obtained by the deposition of the nanosheets onto silicon substrates, as shown in FIG. 3g-i. The corresponding height-profiles are shown by the two step-heights from the substrate in FIG. 3j-1. For CeO₂₋ₓ, these are ˜1.1 nm and ˜1.2 nm, indicating that the nanosheets are of two unit-cell thickness (CeO₂ unit cell=0.54 nm). The thicknesses of the TiO₂₋ₓ and ZrO₂₋ₓ nanosheets were measured to be ˜10.0 nm and ˜1.8 nm, respectively, indicating thicknesses of 20-40 and 3-4 unit cells, respectively. The relatively larger thickness of the TiO₂₋ₓ nanosheet is likely to be due to the poor packing arising from the anisotropy of the tetragonal anatase, while the thin ZrO₂₋ₓ nanosheet probably resulted from the effectively equiaxed lattice. These data suggest that self-assembled metal oxides of equiaxed or possibly highly anisotropic and hence self-aligned nanostructures are more likely to yield ultrathin nanosheets.

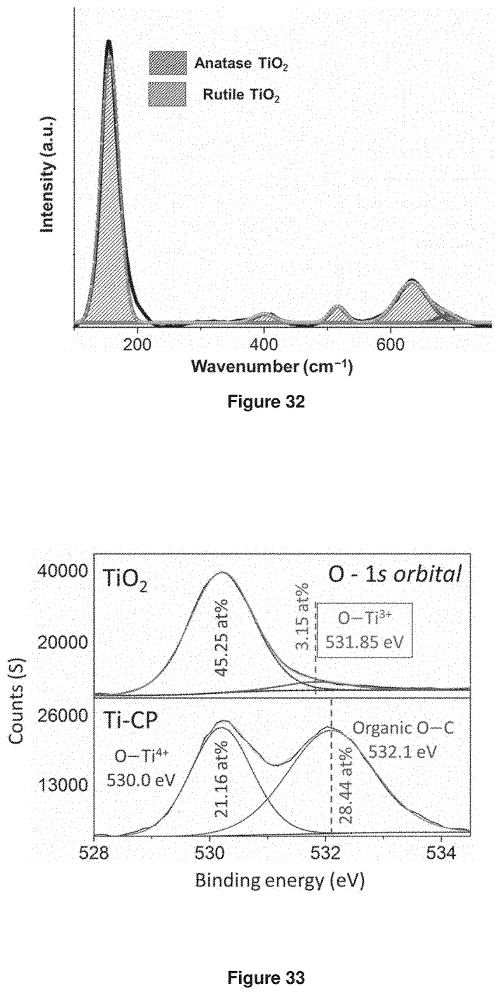

Raman spectra collected from TiO₂ nanosheet is shown in FIG. 32. According to the group theory, there are four predominant peaks attributed to TiO₂ with Raman-active modes of Eg (˜144 cm⁻¹), B₁ g (˜397 cm⁻¹), B₁ g/A1 g (˜516 cm⁻¹) and Eg (˜639 cm⁻¹). Therefore, Raman spectra of the sample show anatase (tetragonal) TiO₂, however slight shifts are observed in the positions of the assigned peaks. Particularly, the peak with the highest intensity is blue-shifted to 153 cm⁻¹. Similarly, the B₁ g and Eg bands also appeared at positions different from the expected frequencies of 397 and 639 cm⁻¹, respectively. The asymmetric broadening and the observed shifts of the peaks can be explained by the phonon confinement phenomenon, which occurs by decreasing the crystal size to nano-scale. The nanosheets in this work have a holey structure composed of nanosized crystallites with diameters of 2-4 nm (shown by the high-resolution TEM images), which can justify these slight shifts. Furthermore, a minor wide peak positioned at 690 cm⁻¹ can be attributed to rutile TiO₂₋ₓ.

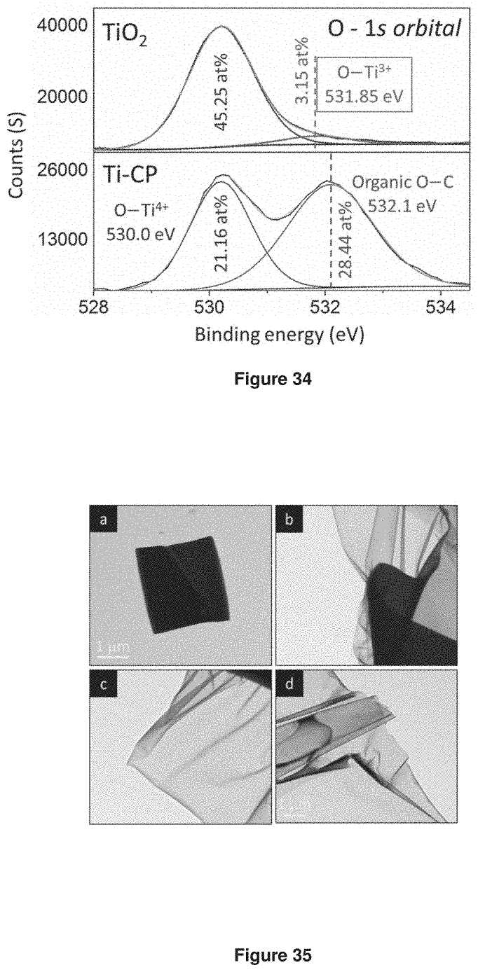

The XPS peak related to the is orbital of carbon for both Ti-CP and TiO₂ are shown in FIG. 33. The peak positioned at 248.8 eV is attributed to the C—C bond of either the sample composition or the adsorbed contaminant on the surface of the sample. The peak positioned at 286 eV is ascribed to the C—O—C bond of formic acetate, the concentration of which is measured to be 9.70 at % by calculating the corresponding peak area. However, this amount dropped to only 2.20 at % by transformation into TiO₂ nanosheet. Further, the peak at 288.5 eV corresponds to the O—C═O bond of formic acid, the atomic percentage of which decreases by ˜13 times from 10.36 at % in Ti-CP to 0.80 at % for TiO₂. The removal of formic organic linker through TiO₂ fabrication is also confirmed by investigation of the oxygen-related XPS peaks (FIG. 34). The peak positioned at 532.1 eV is for is orbital of organic oxygen; however, this peak disappeared in oxygen-related spectra of TiO₂. The two predominant peaks for is orbital of oxygen are O—Ti⁴⁺, and O—Ti³⁺ positioned at 530.0 eV and 531.85 eV, respectively.

The Raman spectra of zirconia nanosheets and the associated fits (reproduced by a set of eight Lorentzian bands corresponding to the most obvious vibrational modes) are shown in FIG. 37. The bands appeared at ˜195 and ˜450 are attributed to the A_{g} vibrational modes of Zr—Zr and Zr—O for monoclinic zirconium oxide, while the broad lines consisting of two peaks at 180 and 240 cm⁻¹ as well as the most predominant peak positioned at 550 cm⁻¹, are assigned to the presence of the cubic phase. Therefore, the Raman data indicates the co-existence of the monoclinic and cubic phases in the zirconia nanosheets.

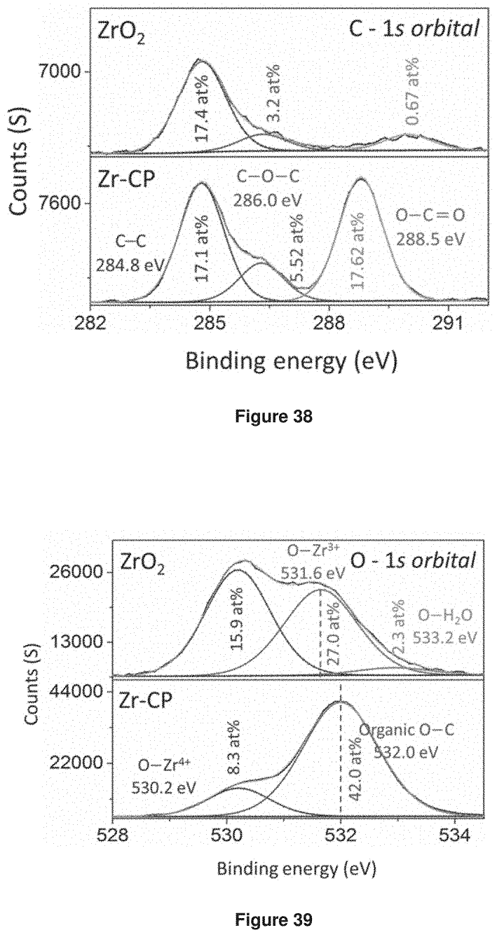

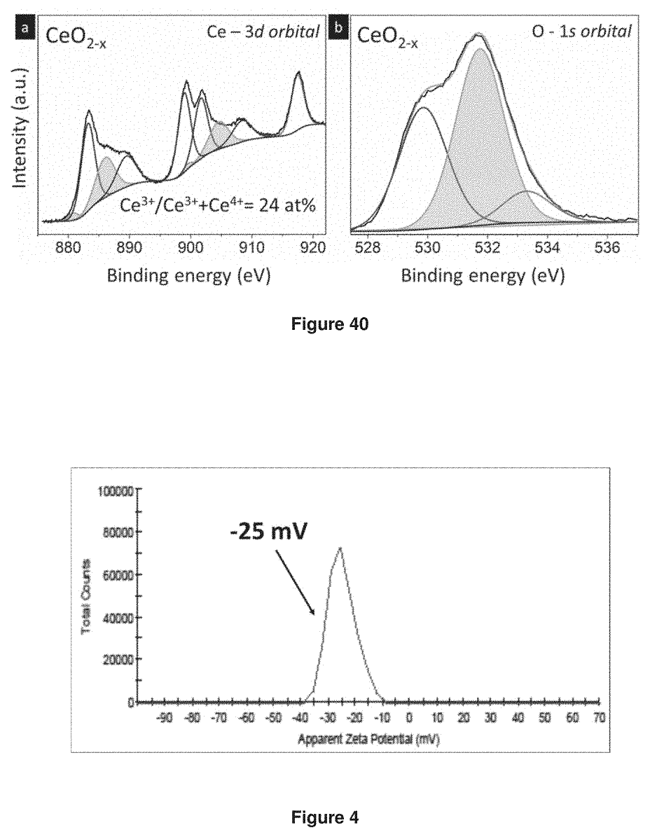

The transformation of Zr-Cp into ZrO₂ has also investigated by XPS analysis, as shown in FIGS. 38 and 39. The XPS peaks related to the is orbital of carbon for both Zr-CP (below) and ZrO₂ (top) are shown in FIG. 38. The unavoidable peak positioned at 248.8 eV is attributed to the C—C bond, which mainly originates from adsorbed contaminant on the surface of the sample. The peaks at 286.0 and 288.5 eV are related to the C—O—C and O—C═O bonds of the formic acetate bonded to Zr. The total atomic percentage of these two peaks was measured to be 23.14 at % for Zr-CP that has decreased to 3.87 at % for ZrO₂. It should be noted that the peak at 286.0 eV can be attributed to the presence of CO₃ owing to the surface bonding between CO₂ of air, owing to the exposure to air, and surface oxygen of the sample and therefore, the surface bonding with CO₂ of air. This bonding is confirmed by the XPS results obtained from is orbital of oxygen as shown in FIG. 39. The organic peak at 532.0 eV in the Zr-CP is removed in ZrO₂ related XPS spectra. Further, the small peak of Zr⁴⁺—O appearing at ˜530 eV for Zr-CP has increased dramatically for ZrO₂. Interestingly, in ZrO₂, the peak at 531.6 eV, which is attributed to O-Zr³⁺ occupies 27.0 at % of the total oxygen concentration in ZrO₂ revealing that defective ZrO₂ is formed. It should be noted that the peak positioned at 533.2 eV is related to the oxygen of adsorbed water, as reported previously.

### Example 5: Synthesis of Heterostructures

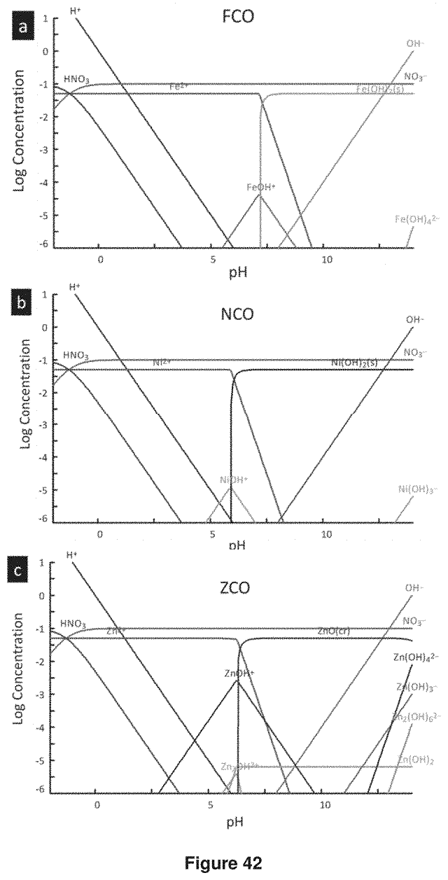

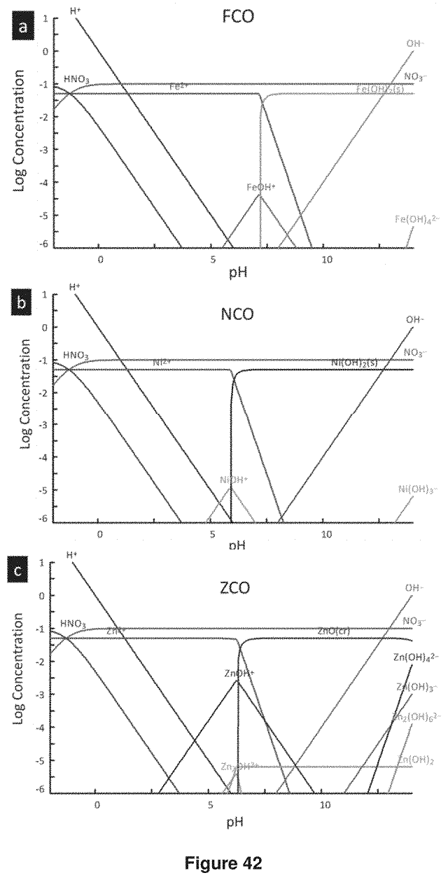

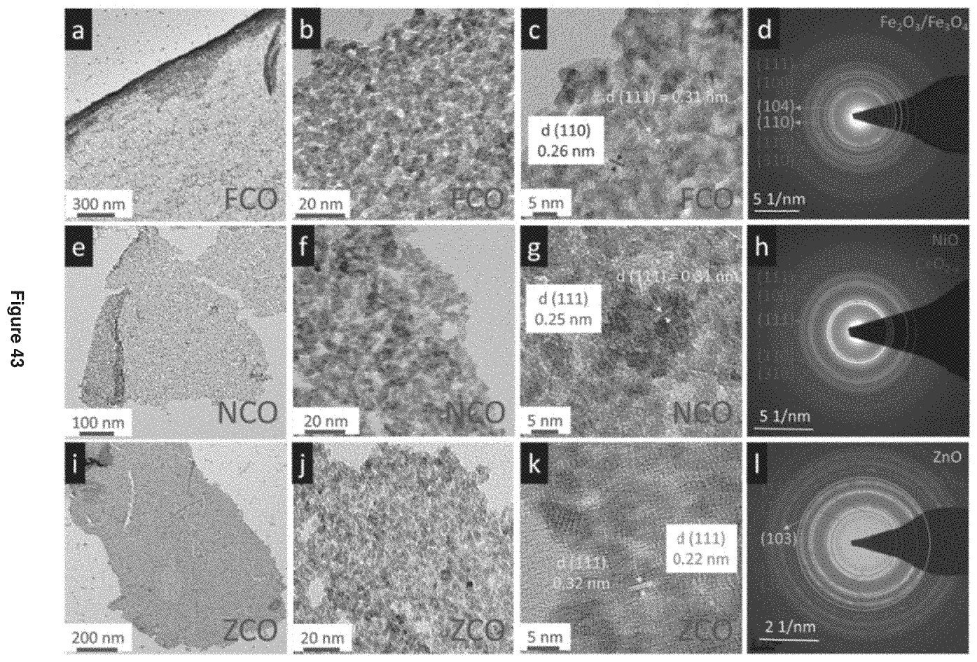

The applicability for holey CeO₂₋ₓ nanosheets can be broadened by their use as a template in the fabrication of mixed 0D/2D heterostructures with Fe-, Ni-, and Zn-based transition metal oxide (TMOs) (0D). Using the general synthesis platform, the holey CeO₂₋ₓ nanosheets were dispersed in an aqueous solution (pH=6), which yielded a relatively stable suspension with zeta potential of −25 mV (FIG. 41), which is slightly lower than the threshold of −30 mV for fully stable colloidal system. In addition, considering the speciation diagrams for the transition metal (TM) ions (FIG. 42), the predominant species, within the acidic pH of CeO₂₋ₓ suspension, are expected to be TMⁿ⁺ Therefore, this situation establishes electrostatic attraction between the positively charged metal species and the negatively charged holey nanosheets, thereby providing the mechanism for the assembly of metal species on the nanosheet surfaces. This is confirmed by reductions in the zeta potential for the Fe, Ni, and Zn nanostructure suspensions, respectively. This approach can significantly increase the functionalities of the nanosheets by preventing the layers from stacking during minimization of the interplanar vdW interactions and by maximizing the accessibility of the active sites.

Moreover, the mixed 0D/2D heterostructures can provide sufficient hybridization between the atomic orbitals, resulting in enhanced carrier delocalization at the junction interfaces. The elemental, mineralogical, and crystallographic investigations of the nanostructures were carried out by EDS, laser Raman microspectroscopy, and XRD as shown in FIG. 4.

The formations of the nanostructures were shown by EDS mapping of the nanosheets in FIG. 4a-c revealing a homogenous distribution of 0D TMOs. Further, the coexistence of the TMOs and CeO₂₋ₓ was confirmed by the laser Raman microspectra (FIG. 4d-f). Since the peak for pristine CeO₂ is at 464 cm⁻¹, the large peaks at ˜460 cm⁻¹ for Fe₂O₃/Fe₃O₄—CeO₂₋ₓ(FCO), NiO—CeO₂₋ₓ(NCO), and ZnO—CeO₂₋ₓ(ZCO) (assigned to the F_{2g} vibrational mode for the symmetrical stretching of Ce(IV) and eight surrounding oxygens) indicate red shifts to lower wavenumber consistent with expansive strains arising from V_{O}^{••}. Further, the peak positioned at −600 cm⁻¹ is attributed to the defect induced mode originating from V_{O}^{••}. The peaks at 230 cm⁻¹ in FIG. 4d is assigned to the A1 g vibrational mode of α-Fe₂O₃, while the peaks at 294, 395, and 620 cm-1 correspond to Eg vibrational modes of α-Fe₂O₃. In addition, there are three deconvoluted peaks at 310 (T_{2g}), 538 (T_{2g}), and 680 (A1 g) that can be attributed to the vibrational mode of Fe₃O₄. FIG. 4e illustrates the coexistence of NiO (magenta color peaks) and CeO₂ (grey peaks). The peaks for Eg, one-phonon transverse optical (1T), one-phonon longitudinal optical (1L), and two-phonon transverse optical (2T) vibrational modes of NiO are laid at 287, 380, 560, and 690 cm⁻¹. Deconvolution of the ZCO peaks in FIG. 4f reveals two peaks at 380 cm⁻¹ and 412 cm⁻¹, which are ascribed to the A_{1T} and E_{1T} vibrational modes of ZnO. Further, the peak at 580 cm⁻¹ is assigned to the E1L vibrational mode of ZnO. Similarly, the coexistence of TMOs and CeO₂₋ₓ nanosheets were confirmed by XRD analysis as shown in FIG. 4g-i. Additional data analysis of the nanostructures are provided in FIG. 43.

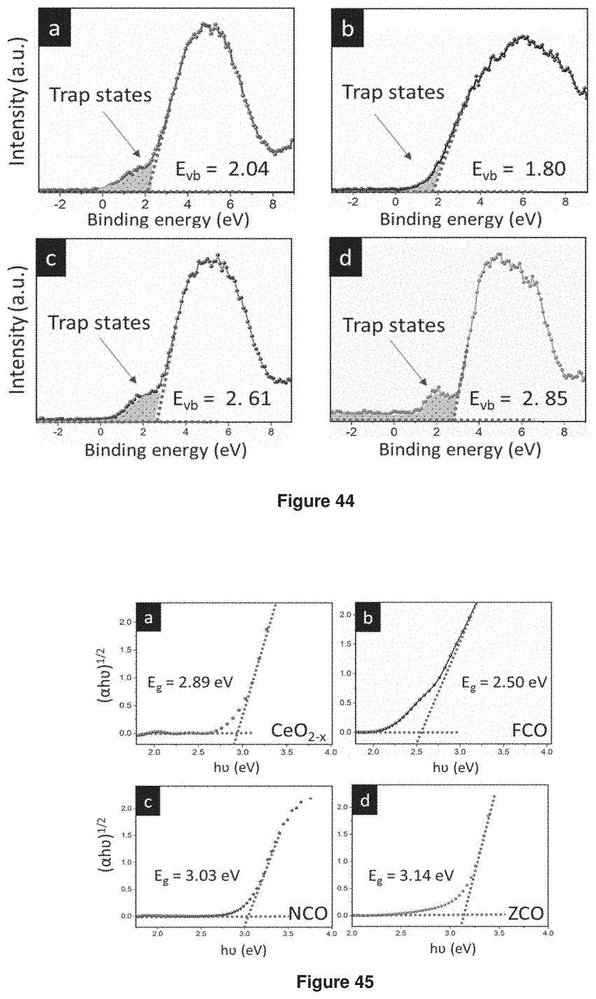

The room temperature photoluminescence (PL) emission of the CeO₂₋ₓ based heterojunction structures are shown in FIG. 46. For FCO, the intensity of the peaks decrease towards zero, indicating minimal electron/hole recombination, owing to the rapid charge carrier separations through very short diffusion routes. The broad emission band positioned at ˜450 nm originates from the surface oxygen vacancies, confirming the high concentration of oxygen vacancies in atomically thin CeO₂₋ₓ holey nanosheet. Adding NiO and ZnO resulted in a considerable reduction of near band edge UV emission peaks while the shift towards the deep-level (DL) emissions within green wavenumber. This reduction can be attributed to the sp-d exchange interactions between the band electrons of the localized d electrons of the Ni²⁺ and Zn²⁺ and CeO₂₋ₓ nanosheet. Further, the high intensity of the PL emission shows increasing defect concentrations in both NCO and ZCO. The increase in the defect concentration is also confirmed by determining the trapping sites from XPS valence band results (FIG. 44). As for NiO, the green emission band at 560 nm is attributed to the defects in the NiO lattice, e.g., Schottky pair defects, interstitial oxygen trapping, and nickel vacancies produced by charge transfer between Ni²⁺ and Ni³⁺ ions. For ZCO, the small and broad emission peaks positioned at 390 nm is attributed to the recombination of the free excitons through an exciton-exciton collision process, which is insignificant for all the heterojunction nanostructures. The weak and broad blue emission band at ˜460 nm is a deep level emission (DLE) originating from the oxygen vacancies or interstitial zinc ions of ZnO nanomaterials. A broad green emission band was observed at 550 nm for all ZnO nanomaterials which may be ascribed to the existence of defects such as singly ionized oxygen vacancies.

### Example 6: Formation of Nanostructures with Unique Morphologies by Controllable Disassembly/Reassembly of Metal Coordination Polymers

The Ce-CP demonstrates environmental stability during long-term exposure. In contrast, the instability of the Ce-CP in polar solvents results in its rapid dissociation. Upon controlled removal of the solvent, ultrafine crystallites of the CP are reassembled to form unique nanostructures. The great lability deriving from the weak electrostatic bonding between the cation and the organic linker in unstable CPs provides a platform for easy and controllable destruction/reconstruction of CP crystallites to form previously unobserved CeO₂₋ₓ nanostructures. These nanostructures are prepared by varying processing parameters, including solvent type, solute concentration, temperature (T), and time (t). Subsequent post-oxidation in air or aging in NaOH basic solution transforms the Ce-CP nanostructures to analogue CeO₂₋ₓ nanostructures. The feasibility, high yield, and adaptability of the disclosed approach, in which the CP acts exclusively as a precursor enables the large-scale synthesis of functional CeO₂₋ₓ nanostructures, such as extremely thin nanosheets (see FIG. 48 u-x) and 2D-3D scaffolds (see FIG. 79), despite the fact that CeO₂ is an intrinsically non-stratified material.

By tuning the highly weak bonds between the metal ion center and coordinated linkers, various metal oxide (MO) architectures can be obtained from a single metal coordination polymer precursor. The success of this method is confirmed by the synthesis of a new and unstable cerium-based coordination polymer (Ce-CP) that can undergo controllable disassembly/reassembly in a polar solvent (ethanol). This allows for the formation of distinctive Ce-CP nanostructures through control of the kinetics of the reassembly process. Post treatment of the Ce-CP nanostructures by low-temperature pyrolysis and/or ageing in an alkaline solution resulted in the formation of defect-rich CeO₂₋ₓ in the form of 2D and 3D nanostructures. This approach provides a rapid, template-free, precisely controllable, and economical approach to synthesise MCPs of specific architectures.

### Electrochemical Fabrication of Ce-CP

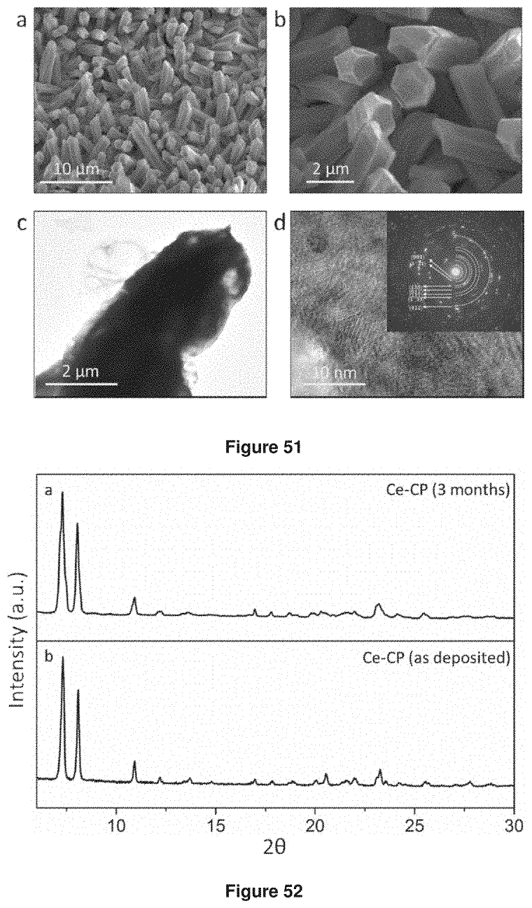

To synthesise the CeO₂₋ₓ nanostructures, the Ce-CP precursor was fabricated as described herein. The schematic of the synthesis process (FIG. 50(a)) indicates that free-standing Ce-CP hexagonal rods are grown on fluorine-doped tin oxide (FTO) substrate. This is shown by SEM images as indicated in FIG. 51. The Ce-CP rods were synthesised under anodic electrochemical current at an aqueous solution and within the oxygen evolution region. One main factor in deposition of the Ce-CP precursor was the application of a high current density within the water oxidation range such that vigorous oxygen bubbling results in an oxidised atmosphere and the formation of acidic pH both at the surface of the working electrode and its vicinity.

The Ce-CP structure was analysed for its stability and it was observed to be fairly stable upon exposure to air for 90 days after the deposition as determined from the XRD patterns of the corresponding samples (FIG. 52). However, the Ce-CP exhibited high instability on exposure to ethanol, which is a polar solvent, and this is shown in FIG. 50(b), step 1.

The simplified molecular structure of the hexagonal Ce-CP rods consists of eightfold-coordinated cerium ions (FIG. 50(c)), where the coordinating oxygen ions are linked by trichloroacetic acetate (TCA) ligands (four), hydroxyl ions (two), and water molecules (two). Additionally, cerium ions are bridged together by covalent bonding with carboxylic groups of the TCA ions, hence forming a two-dimensional (2D) substructure. However, there are weak electrostatic interactions at the interlayer spaces of the 2D Ce-TCA substructure leading to the formation of a stratified structure (FIG. 1A).

The Ce-CP structure, upon exposure to ethanol is disassembled readily forming a pale-yellow transparent solution (FIG. 50(d), (e)). The high instability and resultant rapid disassembly of the Ce-CP is largely due to the retention of the Ce ions in the 4+ valence state. From thermodynamic perspective, the Ce⁴⁺ ion is of higher field strength compared to the Ce³⁺ ion. Consequently, as shown in the Pourbaix diagram (FIG. 7), Ce⁴⁺ has a greater tendency to attract surrounding OH⁻, even in acidic pH, while Ce³⁺ tends to remain in the cationic state.

As discussed herein, the aqueous solution conditions that were used to fabricate the Ce-CP resulted in highly acidic conditions of pH<2.3, which yielded Ce(OH)₂²⁺ as the predominant species. From the relevant speciation diagram, it is seen that this species is a solute that is stable at these pH values but becomes unstable at higher values. Further, the Pourbaix diagram (FIG. 7) showed that Ce(OH)₂²⁺ exists only in the region of water instability, so its presence requires the application of an external bias and suitably low pH. Application of external bias causes exit from water stability range, resulting in rapid proton formation and local pH decrease. Therefore, Ce(OH)₂²⁺ is not formed under typical aqueous processing conditions, which are used in the present work. The coexistence of unsaturated coordination bonds in positively-charged Ce(OH)₂²⁺ along with the negatively-charged bidentate TCA as the organic linker results in the formation of Ce—CP with a unique layered structure. This aqueous chemistry of Ce⁴⁺ differentiates it from that of Ce³⁺, for which there are coordination polymers that cannot be disassembled/reassembled.

In the recrystallisation of the Ce-CP from ethanol, this local bonding configuration and thus the presence of Ce⁴⁺ is retained, thereby enabling the re-formation of the Ce-CP (FIGS. 50(f), (g)). The design of the final architecture can be tailored through control of the kinetics of the solvent evaporation and the concentration of the Ce-CP solute. This novel technique yields a precise controllable assembly of nanostructures at room temperature or at even lower temperatures without using a template. This method can thus be used for form unique architectures that are very difficult to form through pre-existing techniques.

### 2D Ce-CP Nanosheets and Derived Holey CeO₂₋ₓ by Formation of Ce-CP Monolayer at Ethanol/Air Interface

There has been very limited work on the fabrication of cerium-based holey nanosheets, and more importantly, such structures with controllable thicknesses.

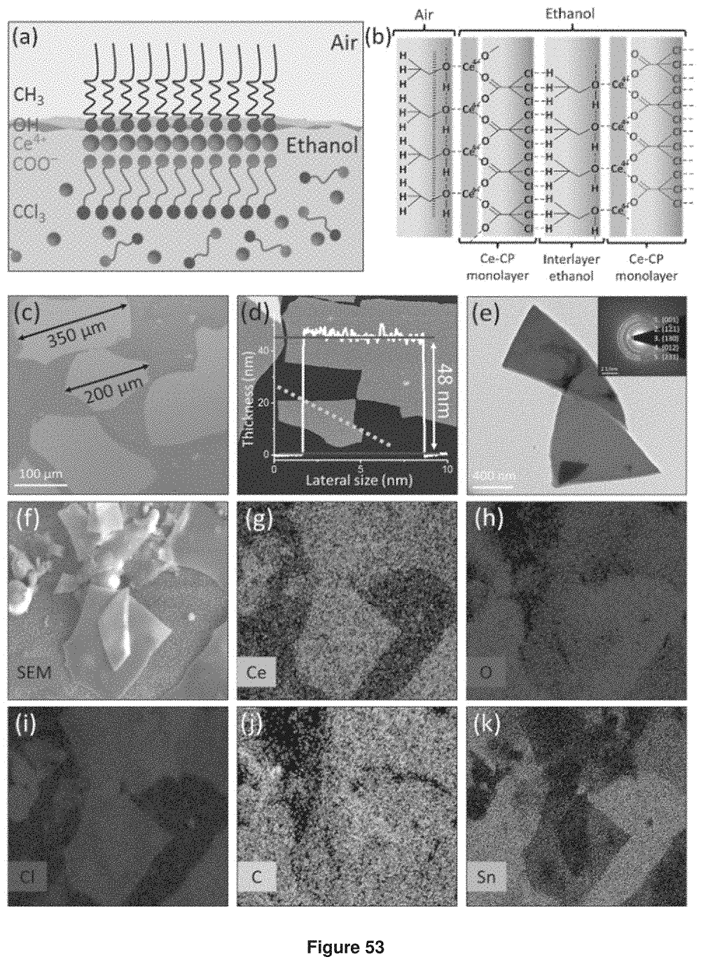

Here, holey ultrathin CeO₂₋ₓ nanosheets with various thicknesses were fabricated successfully by imposing the conditions of slow kinetics of ethanol vaporisation at the low temperature of −10° C. and vapour pressure (VP) of 0.744 kPa. As illustrated in the schematic of a Ce-CP monolayer in FIG. 53(a), these conditions resulted in the formation of individual Ce-CP layers by effectively Langmuir-Blodgett deposition.

In contrast to the work by Wang et al., which used separate solvent and surfactant, the mechanism illustrated in FIG. 53(b) involves surface-assembly of Ce-CP at the ethanol/air interface, where ethanol shows dual functionality as both the solvent and surfactant in this bottom-up 2D process. The aligned projection of the positively-charged hydrophobic —CH₃ groups of ethanol in air establishes a negatively-charged layer consisting of hydrophilic —OH groups of ethanol at the surface. The formation of this layer provides the polar attraction to Ce⁴⁺ ions in solution and thus forms the basis for the development of a cerium-enriched electrostatic double layer. The commensurately aligned —COO⁻ groups attached to the Ce⁴⁺ each contain a negative hydrophobic tail of a —CCl₃ group, the layer of which terminates the Ce-CP monolayer. This terminal layer provides the structural and charge neutrality requirements for electrostatic bonding to the positive —CH₃ groups of ethanol on the opposite terminal layer of the Ce-CP monolayer. Continual evaporation of ethanol provides the driving force for the migration of more Ce⁴⁺ ions toward the surface irrespective of whether the monolayer is permeable or not.

In this way, multiple monolayers can stack together to form sheets with a wide range of thicknesses. This is shown in FIG. 54, where Ce-CP sheets with varying thickness ranging from extremely thin (10 nm) to thick (100 nm) were synthesised during reassembly over 6-72 h. The variation of thickness as a function of evaporation time is plotted in FIG. 54 providing a semi-linear trend for the controllable fabrication of nanosheets with precisely tailored thickness. Further, changes in the Ce-CP concentration, as a precursor, at constant reassembly time of 48 h results in the formation of Ce-CP nanosheets with different thicknesses (FIG. 55).

FIG. 53(c) shows the optical image of the fragmented Ce-CP nanosheets with lateral sizes of a few hundred microns. FIG. 53(d) shows an AFM image of a representative nanosheet collected from the ethanol/air interface after 48 h of ethanol evaporation at −10° C. The associated height profile shown in the inset of FIG. 53(d) revealed a consistent thickness of ˜48 nm. The TEM and corresponding selected area diffraction (SAED) patterns confirm the presence of Ce-CP nanosheets with polycrystalline structure, as illustrated in FIG. 53(e) and the corresponding inset, respectively. Elemental mapping done by energy dispersive spectroscopy (EDS) (FIG. 53(f)-(k)) shows the predominant elements to be Ce and Cl. These nanosheets can be transferred easily to a glass substrate using van der Waals exfoliation technique.

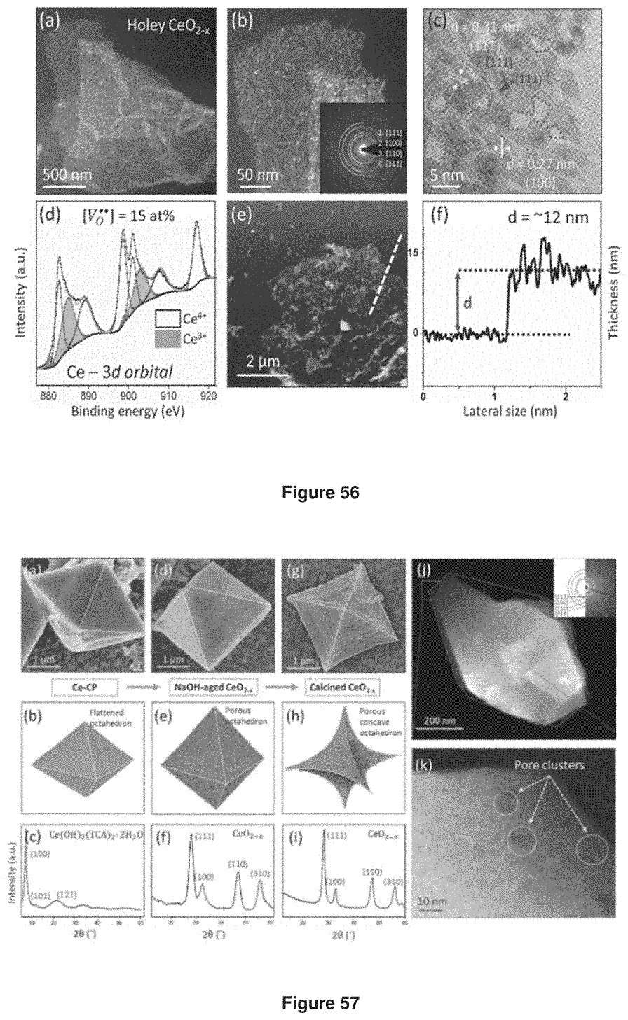

The Ce-CP transformation into CeO₂₋ₓ was carried out by aging the Ce-CP nanosheets in strongly basic solution (6 M NaOH) at room temperature followed by heating at 200° C. As a result, the 2D morphology was retained along with widespread nanohole formation. FIGS. 56(a) and (b) show high angle annular dark-field (HAADF) images of the holey CeO₂₋ₓ nanosheet. The polycrystalline nature of the CeO₂₋ₓ is confirmed by the SAED pattern in inset (FIG. 56(b)). The high-resolution TEM (HRTEM) image of the nanosheet (FIG. 56(c)) illustrates crystallites with sizes in the ranges of 4-8 nm and intercrystallite holes of up to 10 nm. In addition, there are strong chemical bonds between the single crystallites owing to the cross-fringed lattices. FIG. 56(d) shows the XPS spectra of the holey CeO₂₋ₓ nanosheet that indicates the coexistence of both Ce³⁺ and Ce⁴⁺ oxidation states in the CeO₂₋ₓ. As discussed above, the presence of Ce³⁺ reflects the oxygen vacancy defects (V_{O}^{••}), which is considered as an active site in catalysts. The concentration of oxygen vacancies ([V_{O}^{••}]) was quantified indirectly from the amount of Ce³⁺ and this is discussed later. FIGS. 56(e) and (f) show the AFM image (e) and the corresponding height profile (f) of a highly porous CeO₂₋ₓ nanosheet derived from a Ce-CP nanosheet collected after 10 h of evaporation.

### 3D Ce-CP Hollow Pseudo-Octahedra and Derived CeO₂₋ₓ

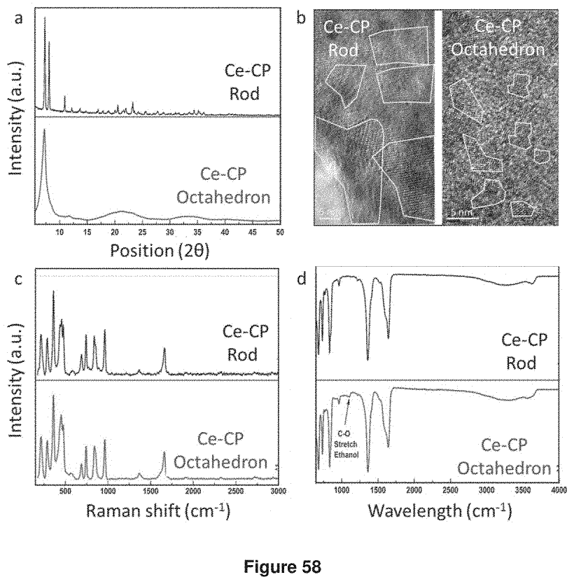

The role of evaporation kinetics was investigated by rapid recrystallisation of the Ce-CP at room temperature while the concentration remained unchanged ([Ce-CP]=˜8 M). Ce-CP can form a free-standing Ce-CP pseudo-octahedron. FIG. 57(a) shows the SEM image of a free-standing Ce-CP pseudo-octahedron. The pseudo-octahedra with variable c axis length, terminated by positive and negative pyramids, shown in FIG. 57(b), is a common crystal form for minerals crystallising in the monoclinic system. The XRD pattern of the Ce-CP octahedral is identical to that of the Ce-CP rods (FIG. 58(a)), confirming that the crystal structure remained unchanged and is unaffected by the disassembly/reassembly process. However, the peaks for the hollow pseudo-octahedra were broadened relative to those of the rod. The difference in full-width of half-maximum (FWHM) of the XRD patterns can be rationalised by smaller crystallite size of the hollow pseudo-octahedra, relative to the Ce-CP rod/tube (FIG. 58(b)). For further confirmation, the identical chemical structures of the Ce-CP tube and pseudo-octahedra were shown by laser Raman microspectroscopy and Fourier transform infrared spectroscopy (FTIR) (FIG. 58(c), (d)).

The transformation into CeO₂₋ₓ without morphological change was carried out by aging/converting the pseudo-octahedra in the concentrated NaOH solution at room temperature. This is illustrated by the SEM image and the corresponding schematic in FIGS. 57(d) and (e), respectively.

The XRD pattern of the CeO₂₋ₓ derived from the Ce-CP (FIG. 57(f)) was indexed to the cubic fluorite structure of CeO₂, space group Fm3m. Generally, the transformation of a CP into a metal oxide is attributed to the replacement of weakly-bonded organic linkers by the OH⁻ and/or H₂O in aqueous solution. For Ce-CP in aqueous solution, the relatively high field strength of Ce⁴⁺ enhances its ability to form Ce(OH)₄, which readily converts to CeO₂₋ₓ upon drying. The transformation can be followed by pyrolysis at temperature of >200° C. Although this results in concave distortion of the facets owing to removal of the residual OH⁻ and H₂O molecules (FIG. 57(g), (h)), it results in increasing crystallinity (FIG. 57(i)). Further, from the SEM image of the CeO₂₋ₓ pseudo-octahedron, it was revealed that there are pores formed on the structures (shown by magenta circles in FIG. 57(g)). This is confirmed by the dark field HRTEM imaging (FIG. 57(j), (k)), in which the pore clusters of ˜10 nm size is identified. The diffuse rings in the selected area diffraction (SAED) pattern (FIG. 57(i) inset) show the randomly orientated structure of the polycrystalline CeO₂₋ₓ. The BET surface area of the hollow pseudo-octahedra was measured to be 47.18 m² g⁻¹ with a pore size of 6.86 nm and pore volume of 0.42 cm³/g⁻¹.

A second key factor controlling the structural reassembly is the Ce-CP concentration. In principle, the concentrations of the principal ions in solution determine the supersaturation factor (S) according to the following equation:

$$S = \sqrt{\frac{{C}_{Ce} \times {( {C}_{TCA} )}^{δ}}{{K}_{sp}}}$$

where C_{Ce}, C_{TCA}, Kₛₚ, δ are defined as the concentrations of cerium cations and dissociated TCA anions, solubility product constant, and number of ions in the complex anion (TCA), respectively. Increasing the value of S results in a shift in the crystallisation towards 3D structure, while lower value of S leads to formation of structures with lower dimensions, e.g., 2D. According to the constant Ks, for the Ce-CP, increasing the Ce-CP concentration is expected to lead to the formation of 3D architectures, as observed in the case of the pseudo-octahedra.

### 3D Ce-CP Hollow Spheres and Derived CeO₂₋ₓ

Another critical factor is the electrolytic dissociation (u) of the Ce-CP, which represents the dissociation amount of the Ce-CP. This value is considered to be ˜1 owing to full disassembly of the Ce-CP in ethanol.

α=(C_{TCA}/C_{Ce-CP})

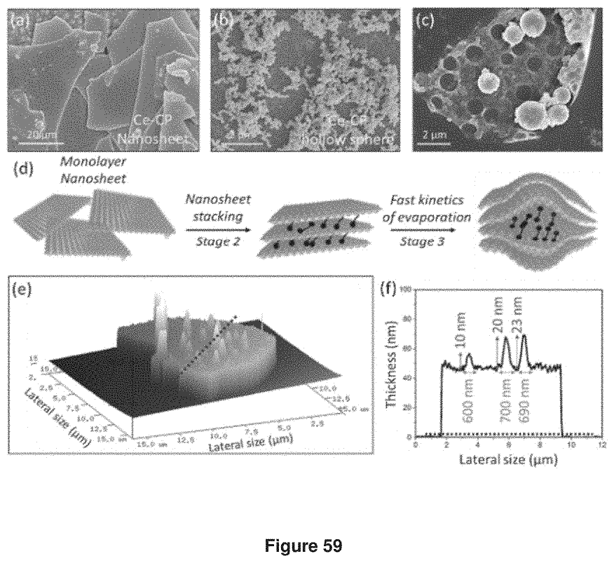

The effect of S was foreshadowed by focusing on the kinetics of nucleation/growth by tailoring the vapour pressure of the ethanol solvent. This was shown previously through the fabrication of different morphologies at 25° C. (pseudo-octahedra) and −10° C. (holey nanosheets). That is, the significantly different VPs of ethanol at these two temperatures, i.e., 7.830 kPa and 0.744 kPa, respectively, indicate the presence of significant Ce-CP concentration gradients during evaporation. Consequently, the intermediate temperature of 0° C. (VP=1.568 kPa) was selected as the basis for the examination of the effect of concentration and the corresponding results are illustrated in FIG. 59. FIGS. 59(a) and (b) show that increasing the [Ce-CP] by four times (from 4 M to 16 M) causes the resultant morphologies to change from nanosheets to purely hollow spheres. This transformation is confirmed by SEM image of the spheres being liberated from the stacked nanosheets (FIG. 59(c)).

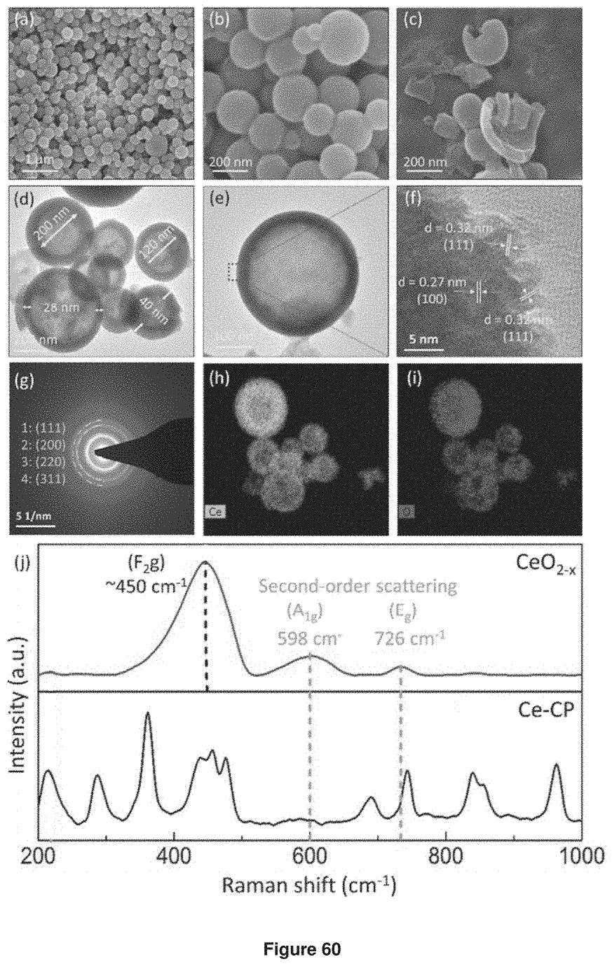

The proposed formation mechanism of the spheres is based on bloating of the nanosheets during the evaporation of interlayer ethanol, and this is schematically shown in FIG. 59(d). To confirm the proposed mechanism, the experimental conditions were designed to accelerate the evaporation responsible for the formation of hemispheres prior to sphere formation and detachment. The 3D AFM image and corresponding height profile are shown in FIGS. 59(e) and (f), respectively. The hemispheres were of diameters ˜600-700 nm (heights ˜10-25 nm), which are larger than those of the spheres at ˜200-400 nm, as shown in FIG. 60; this is attributed to the gradual contraction of the former during the formation of the latter.

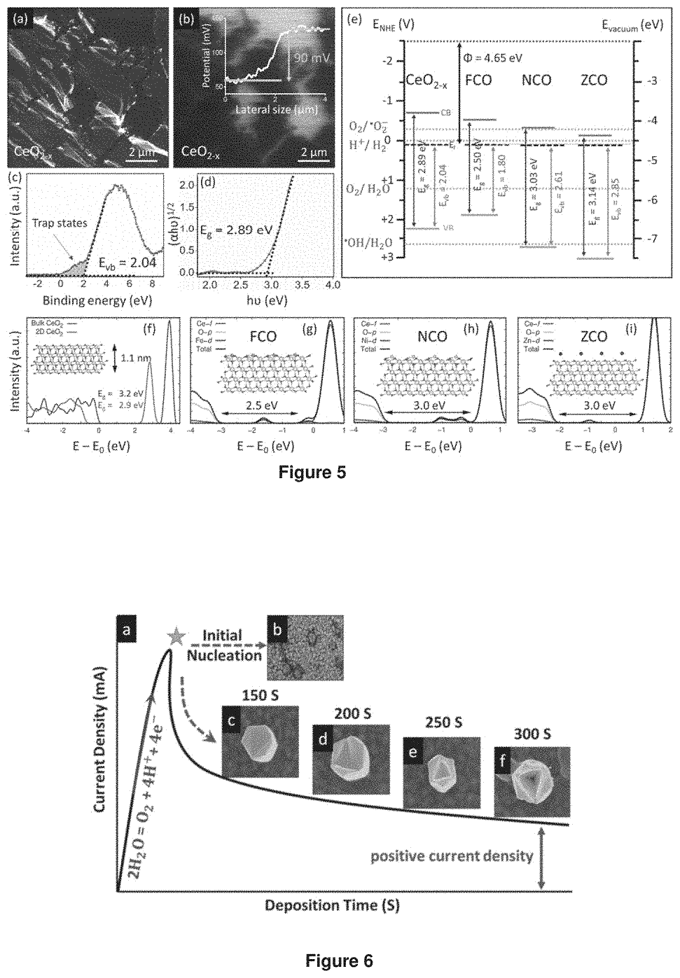

Similarly, the NaOH ageing and pyrolysis at 200° C. were used to transform the Ce-CP into CeO₂₋ₓ spheres. The CeO₂₋ₓ hollow spheres had sizes between 200 and 400 nm, with a wall thicknesses being in the range of −28-40 nm. The SEM images of the CeO₂₋ₓ are shown in FIG. 5a-c revealing the hollow spheres with sizes between 200 and 400 nm. The TEM images in FIGS. 60(d) and (e) show the hollow structure of the CeO₂₋ₓ, while the wall thicknesses of the spheres were in the range of −28-40 nm. These thicknesses are assumed to be approximately half the thickness of the original nanosheets. HRTEM image of an individual hollow sphere (FIG. 60(d)) is shown in FIG. 60(e) in which the crystallites with exposed facets of (111) and (100) are identified. The SAED pattern of the hollow spheres, as shown in FIG. 60(g), was indexed to CeO₂ and the rings confirms the polycrystalline structure. Further, FIGS. 60(h) and (i) show EDS mapping of Ce and O in the CeO₂₋ₓ hollow spheres.

FIG. 60(j) shows Raman spectra of the Ce-CP and the effects of aging and heating processes on the CeO₂₋ₓ derived Ce-CP. After the NaOH aging, the peak at 455 cm⁻¹ was indexed to the F₂ g vibration mode of Ce and O. However, the asymmetric nature and red shift of the peak is attributed to the presence of the V_{O}^{••} in the structure. This is confirmed by the three broad lower intensity peaks, which are indicative of charge-compensating (V_{O}^{••}). After heating at 200° C., the single narrow peak indicates relatively well crystallised CeO₂₋ₓ, which shows a blue shift (higher values) in the F₂ g peak positioned at 464 cm⁻¹, resulting from residual compression and annihilation of the V_{O}^{••}.

### Overall Ce-CP Formation Mechanism

The preparation of the disclosed nanostructure architectures and resulting performances are likely to be contingent upon the use of unstable CPs. The disclosed approach generally offers rapid but variable disassembly/reassembly kinetics through the use of different solvents at room temperature to generate new nanostructures. For example, immersion of the Ce-CP in deionized water results in gradual Ce-CP exfoliation, as demonstrated by the ex-situ TEM imaging and schematics in FIG. 1-h. The exfoliation occurs due to the intercalation of the water molecule between the terminal Cl ions on the Ce-CP nanosheets. Further, deionized water does not act as a solvent owing to the difference in polarity indices. Applying the same method to the Zr-CP and Ti-CP gave the same outcomes, thus highlighting the universality of the disclosed approach.

In contrast, a weakly polar solvent such as ethanol causes very rapid disassembly/reassembly of Ce-CP. This solubility indicates that the Ce-CP is of similar medium polarity as the solvent. The propensity for very fast structural change in the Ce-CP at room temperature is shown by the disassembly of the tubular Ce-CP nanostructure in 1.5 min as well as the rapid reassembly in the form of octahedra during ethanol evaporation. Additionally, in-situ Raman spectra revealed alterations in vibrational modes of the structural bonds during disassembly/reassembly of the Ce-CP. The Raman spectra after 360 s shows no trace of ethanol, which confirms that ethanol provides only a medium for disassembly and reassembly of the Ce-CP nanostructures. Such behaviour also was observed for the MOF-5 nanostructure, where different experimental conditions resulted in a variety of ZnO nanostructures.

The disassembly kinetics of the unstable CPs may be enabled by: (i) high cation valence and its associated high field strength, which favours hydroxide formation at high pH; (ii) tendency of the linker to protonate in aqueous solvents at low pH, thereby replacing the linker with a hydroxyl group; (iii) linker (monodentate, bidentate, etc.) of low molecular symmetry; and (iv) match between the polarities of the solute and solvent. For Ce-CP, the Ce⁴⁺ has a relatively strong field strength and so it favors bond formation with the hydroxyl group over that for bonding with the monodentate trichloroacetate (TCA) linker; this effectively destabilizes any Ce-TCA bonds. The solvents exhibiting the most rapid kinetics are those that have polarity indices in the range 4.3-5.9, which suggests that the polarity index of the Ce-CP falls within this middle range. Further, the reassembly kinetics of the new nanostructures depend principally on the partial pressure of the solvent, which can be manipulated by temperature and chemical potential. For example, when ethanol is evaporated rapidly at room temperature, hollow octahedra are formed but, if the evaporation is done at 0° C., hollow spheres are formed.

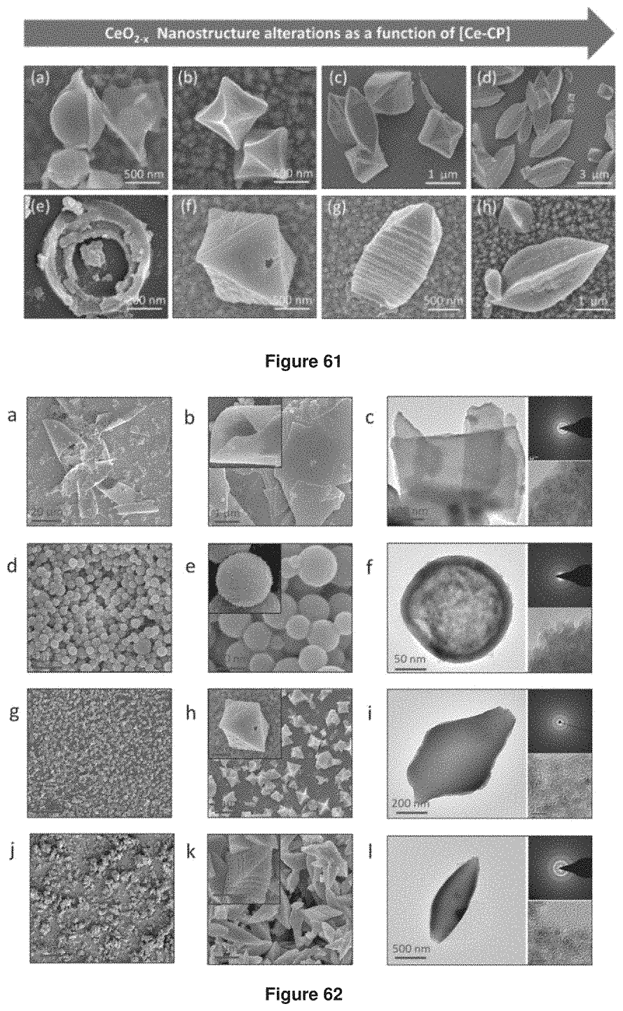

FIG. 61 illustrates various nanostructures obtained as a function of [Ce-CP], where FIG. 4(a-d) show the Ce-CP nanostructure and FIGS. 59 (e-h) show the CeO₂₋ₓ obtained through NaOH ageing and heating at 200° C. An architectural alteration of the Ce-CP and consequently CeO₂₋ₓ as a function of increasing [Ce-CP] follows the order of nanosheets, hollow spheres, hollow pseudo-octahedra, hollow elongated octahedra, and dense leaves.

The model for the stacking of the multiple flat nanosheets suggested in FIG. 53 is supported by the presence of the ridges clearly apparent in FIGS. 61(f), (g) and faintly visible in FIG. 61(h) and FIG. 55. Finally, the dense leaf morphology shown in FIG. 61(h) is formed as a result of the collapse of the elongated octahedral morphology shown in FIG. 61(g). This density derives from the greater [Ce] and the consequently reduced diffusion distance. All the nanostructures in FIG. 61 were generated at low temperature (25° C.) and thus the driving forces for diffusion were low. This favoured the formation of polycrystalline structures rather than single-crystals. Consequently, the flexibility in generating the various nanostructures suggests structural alteration through low-energy displacive rather than high-energy reconstructive phase transformations.

Further morphological analyses of the CeO₂₋ₓ are provided in FIG. 62. Identification of the profusion of single CeO₂₋ₓ pyramids in FIG. 62(h), in combination with the ridges present in the pseudo-octahedra suggest that these forms were generated from the mated hemispheres still being attached to diametral nanosheets (FIG. 61(a)). The process can be proposed to occur by early faceting via planarisation of the rounded hemispheres, where the ridges are formed from the fracture of the flexible Ce-CP monolayers. As suggested in FIG. 61(a), the pyramids are formed before separation from the nanosheets owing to the presence of the maximal diametral stress at the circle of greatest sheet misalignment. While the individual pyramids would have formed by complete delamination, the nanosheets and the elongated octahedra are formed by a different mechanism. These structures are likely to have been generated by cyclic evaporation of and backfilling by ethanol when the two hemispheres remained in close proximity, causing chemical gradient fluctuations. Closer inspection of FIG. 61(g) supports this notion in that the central ridges of the elongated octahedra are the most misaligned, suggesting the closure of the two mated pyramids at the final stage of evaporation-condensation.

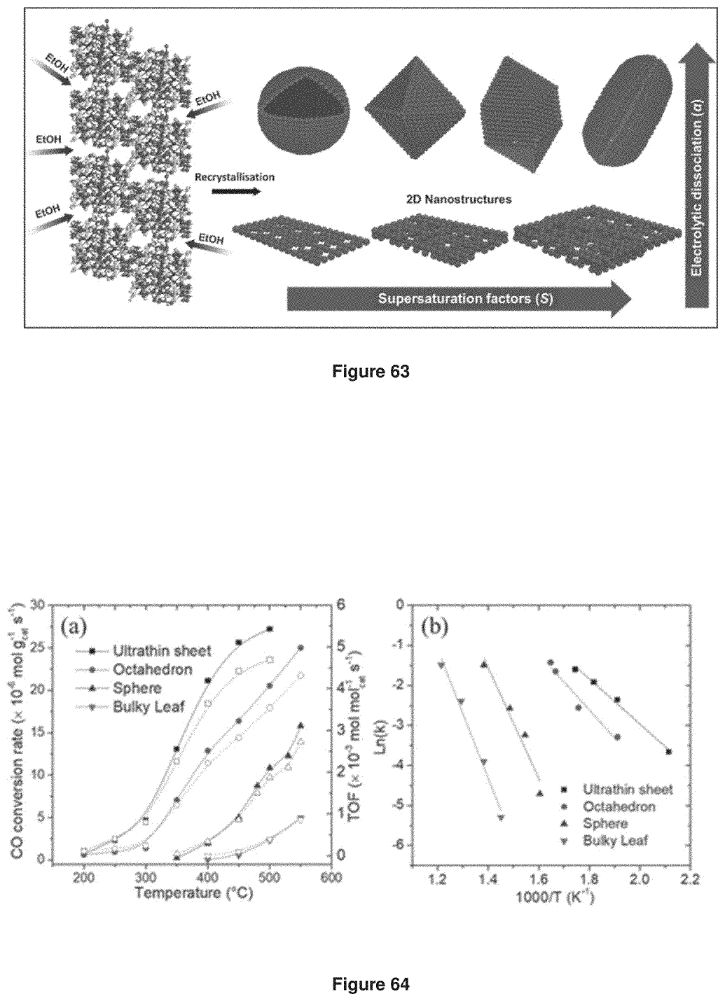

The general formation mechanism for the microstructures is shown in FIG. 63. It is known that recrystallisation of assemblies from an electrolyte containing both component cations and anions is determined by electrolytic dissociation (α) and supersaturation factor (S), both of which are described in the preceding equations.² In terms of α, the solvent ethanol, which has a high dissociation degree, and the solute Ce-CP are single variables and so the electrolytic dissociation factor in principle is fixed. Despite this, the α during recrystallisation was varied through temperature variation, which altered the ethanol evaporation rate. Similarly, in terms of S, the changes in Ce-CP concentration during evaporation also resulted in variations in the S value. Consequently, the formation of polycrystalline 2D and 3D structures depends on the synergetic control of both the α and S factors. Such control through a single experimental variable allows the systematic and precise variation of the morphology from 2D to 3D. More specifically, evaporation kinetics characterised by low α and S factors result in the formation of ultrathin 2D Ce-CP nanosheets. When a low α factor is retained but the S factor is increased, increasing nanosheet thickness occurs. When the α factor is increased through evaporation at room temperature, increasing S results in alteration to 3D structures in the progression hollow spheres, hollow pseudooctahedra, hollow elongated pseudooctahedra, and finally solid leaf.

Overall, metal-based CP (MCP) processing approach represents a simple, cost-effective, template-free, and low-temperature method (≤25° C.) for the fabrication of metal oxides with unprecedented architectures. This approach involves oxidation of cerium-based MCPs, which allows rapid disassembly/reassembly in the polar solvent ethanol and so yields well-defined holey 2D and hollow 3D CeO₂₋ₓ nanostructures with high functionalities. Fabrication of holey 2D metal oxide with precisely controlled thicknesses was achieved by manipulation of the kinetics of nucleation/growth of the MCPs.

### Example 7: Photocatalytic Activity of Nanostructures

### Photocatalytic Parameters

In order to investigate the photocatalytic parameters of CeO₂₋ₓ and the mixed nanostructures, the corresponding electronic band structures were constructed. Hence XPS, UV-Vis spectrophotometry, and amplitude-modulated kelvin probe force microscopy (AM-KPFM) were used to determine the gaps between the valence bands (VB, orange lines) and the Fermi levels (Ef, black dashed lines), optical indirect band gaps (Eg), and the work functions (Φ). The AFM image in FIG. 5a illustrates the basis for the KPFM result for CeO₂₋ₓ shown in FIG. 5b. There is a significant difference of 90 mV (0.09 eV) potential between that of the silicon substrate (higher potential) and the deposited 1.2 nm thickness CeO₂₋ₓ nanosheet (lower potential). Since the Φ of a platinum/iridium-coated silicon tip was measured to be 4.74 eV (similar to that reported previously), then subtracting 0.09 eV gives a Φ for CeO₂₋ₓ of 4.65 eV. The XPS plot for the valence band of CeO₂₋ₓ is shown in FIG. 5c, where the presence of trapping states within the bandgap is also illustrated. Additionally, the Tauc plot for the Eg is shown in FIG. 5d. These data and those for Fe₂O₃/Fe₃O₄—CeO₂₋ₓ nanostructure (FCO), NCO, and ZCO (FIGS. 44 and 45) were used to construct the electronic energy level diagrams shown in FIG. 5e.

The preceding demonstrates that these holey 2D nanostructures offer the dual advantages of rapid charge-carrier diffusion and significant reduction in the Eg from 3.36 eV to 2.89 eV. Further, there is the potential to leverage the effects of midgap trapping states (FIG. 5c) associated with the presence of V_{O}^{••} and V_{Ce}^{″″}, although the positions of the corresponding energy levels do not appear to have been determined. FIG. 5e demonstrates that the photocatalytic capacity for specific chemical reactions can be engineered by modification of the electronic band structure through the creation of nanostructures. For example, FIG. 5e shows that the FCO lowers the Eg to 2.50 eV and positions the CB (green line) for CeO₂₋ₓ above that of Fe₂O₃/Fe₃O₄ but also above the O₂/.O₂⁻ energy level. The reduction of the bandgap significantly increases light absorption and the new CB position of FCO, which is in the proximity of O₂/.O₂⁻, enhances the formation of reactive oxygen species (ROS) by enabling electron transfer from CeO₂₋ₓ to Fe₂O₃/Fe₃O₄. The VB and CB band alignments also suggest that charge transfer of both electrons and holes would be toward Fe₂O₃/Fe₃O₄, hence enhancing charge recombination. However, reduced electron/hole recombination of the mixed 0D/2D heterostructures relative to the CeO₂₋ₓ nanosheet was confirmed by PL spectroscopy (FIG. 46). These data suggest that charge transfer is dominant owing to short diffusion pathways in the nanosheets, rather than electron/hole recombinations.

XPS analyses (FIG. 44) of the NCO and ZCO nanostructure also showed the formation of trapping states. Although the band gaps of NCO and ZCO were increased (FIG. 5e), the CB in NCO and the VB in both NCO and ZCO are positioned appropriately to catalyse the O₂/.O₂⁻ and .OH/H₂O reactions, respectively, thereby enhancing the respective ROS formation. Further, both the VB and CB decrease relative to those for CeO₂₋ₓ, indicating that charge separation would be improved by electron diffusion to the TMO and hole diffusion to the CeO₂₋ₓ.

First-principles calculations based on DFT were performed to characterize further the differences in electronic band structures between CeO₂ nanosheets, bulk CeO₂, and 0D/2D heterostructures. FIG. 5f shows that the band gap of the CeO₂ nanosheets is reduced by ˜10% relative to that of bulk CeO₂, which is in excellent agreement with the experimental result (FIG. 5d). Upon adsorption of transition metal ions, noticeable variations in the band structure of the CeO₂ nanosheets are observed in the form of new electronic states appearing in the band gaps (FIG. 5g-i) and, in one case, the bottom of the conduction band (FIG. 5g); the band gaps are in good agreement with the experimental data (FIG. 5e). The origins of such band structure differences are supported by differences in computed transition metal adsorption energies, which are −10.8 eV (Fe), −3.8 eV (Ni), and −0.1 eV (Zn). Larger charge transfers typically are correlated with more favorable adsorption energies, so different attractive electrostatic interactions lead to significant differences in the amounts of charge that the transition metal ions transfer to the nanosheets (˜2 e− per Fe ion, ˜1 e− per Ni, and ˜0 e− per Zn). These variations suggest a wide range of potential band tuning through the formation of 0D/2D heterostructures using different ions.

The room temperature photoluminescence (PL) emission of the CeO₂₋ₓ based heterojunction structures are shown in FIG. 46. For FCO, the intensity of the peaks decrease towards zero, indicating minimal electron/hole recombination, owing to the rapid charge carrier separations through very short diffusion routes. The broad emission band positioned at ˜450 nm originates from the surface oxygen vacancies, confirming the high concentration of oxygen vacancies in atomically thin CeO₂₋ₓ holey nanosheet. Adding NiO and ZnO resulted in a considerable reduction of near band edge UV emission peaks while the shift towards the deep-level (DL) emissions within green wavenumber. This reduction can be attributed to the sp-d exchange interactions between the band electrons of the localized d electrons of the Ni²⁺ and Zn²⁺ and CeO₂₋ₓ nanosheet. Further, the high intensity of the PL emission shows increasing defect concentrations in both NCO and ZCO. The increase in the defect concentration is also confirmed by determining the trapping sites from XPS valence band results (FIG. 44). As for NiO, the green emission band at 560 nm is attributed to the defects in the NiO lattice, e.g., Schottky pair defects, interstitial oxygen trapping, and nickel vacancies produced by charge transfer between Ni²⁺ and Ni³⁺ ions. For ZCO, the small and broad emission peaks positioned at 390 nm is attributed to the recombination of the free excitons through an exciton-exciton collision process, which is insignificant for all the heterojunction nanostructures. The weak and broad blue emission band at ˜460 nm is a deep level emission (DLE) originating from the oxygen vacancies or interstitial zinc ions of ZnO nanomaterials. A broad green emission band was observed at 550 nm for all ZnO nanomaterials which may be ascribed to the existence of defects such as singly ionized oxygen vacancies.

The functionality of CeO₂₋ₓ nanostructures can be evaluated on the basis of their defect contents (oxygen vacancies V_{Ö}, where there is charge compensation between 2(Ce⁴⁺→Ce³⁺) and V_{Ö}), which were evaluated for representative nanostructures using high-resolution X-ray photoelectron spectroscopy and Raman microspectroscopy. The XPS data show that the concentrations of Ce³⁺ ([Ce³⁺]) for the nanostructures differ according to the architecture, with the highest [V_{Ö}] being for the holey nanosheets (9.5 at %) and the lowest being for the solid rhombohedra (4.0 at %). The Raman data demonstrate that the vacancies in CeO₂₋ₓ cause the peak at ˜462 cm⁻¹, which represents the Ce—O bond, to red shift gradually to lower wavenumbers. Since the extent of the shift is a measure of the defect concentration, then the shifts of these nanostructures are consistent with the [Ce³⁺]. High magnification SEM image of 2D-3D scaffold revealing porous structure of the 3D scaffold comprising flower-like 2D nanolayers. The 2D-3D nanostructure is comprised of small nanocrystallites (<10 nm) that exhibit both strong intergranular bonding as well as gaps, the latter of which increase the exposed facets and thus the number of active sites; the nanosheets exhibit similar nanostructures. It is significant that the HAADF image of the holey nanosheet suggests the presence of Ce vacancies (VCe″″), which, to the best of the inventors' knowledge, have not yet been observed in CeO₂-based materials. Such defects also could indicate Schottky pair formation, although this requires ˜2-3 eV more than for O vacancy formation. HAADF imaging and EELS analysis in STEM mode were conducted while the samples were cooled in-situ to liquid nitrogen temperature, which inhibited the creation of artifact vacancies that possibly caused by the application of high vacuum and/or electron beam irradiation. The EELS data allow the determination of the [V_{Ö}] from the ratio of the M5 (orange) and M4 (green) peaks, where the ratios for minimal [V_{Ö}] (0 at % for stoichiometric CeO_{2.0}) and maximal [V_{Ö}] (25 at % for CeO_{1.5}) are ˜0.9 and ˜1.25, respectively. According to the reported linear relationship between the [Vo] and M5/M4 peak ratios, the [V_{Ö}] of the 2D-3D scaffold and nanosheet structures were measured to be 4.5 and 11 at %, respectively. Such medium to high defect levels in CeO₂ usually are obtained by adding dopants or heat treatment under reducing conditions, which are added complications. The significant concentrations of defects achieved without these indicates that the processing of unstable CPs can yield a wide range of MO nanostructures that are characterized by high defect densities and associated high-level functionalities.

### Catalytic Performance

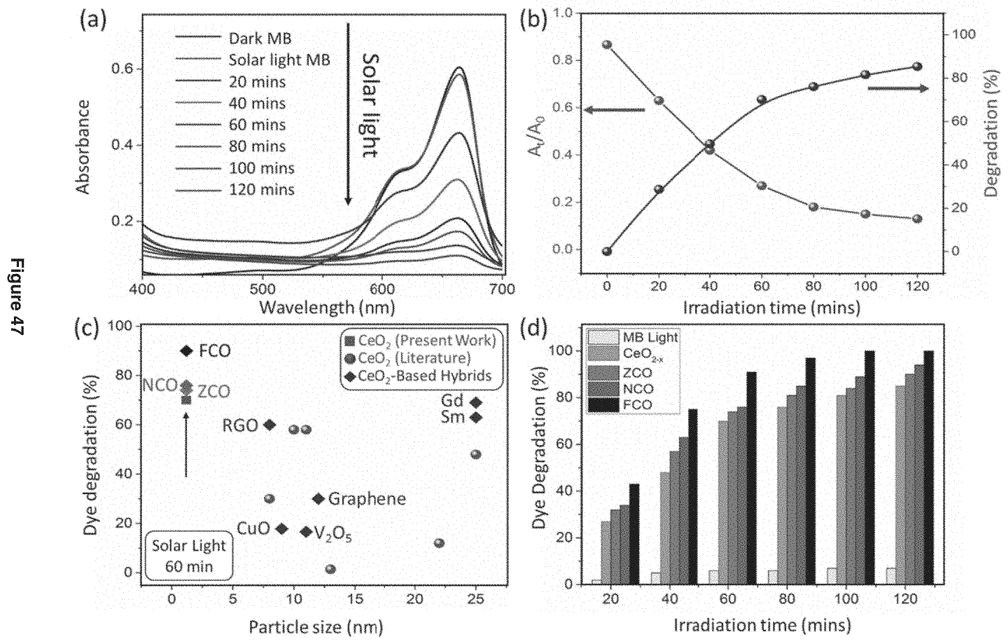

The photocatalytic performance of the samples was assessed by degradation analysis of methylene blue (MB) compound, which is used extensively for photocatalytic analysis, under solar light irradiation. The gradual decrease of intensity of the absorbance peak of MB, which is centred at 664 cm⁻¹, in the presence of the nanosheets was measured. While the holey CeO₂₋ₓ nanosheet exhibits a high dye degradation extent of 85% after 2 h (FIG. 47), the kinetics of the reaction reveals a rate constant (k) as high as 0.024 min⁻¹, which represents the fastest dye degradation by pure CeO₂ reported (Table 4). The 0D/2D heterostructures performed even better, with FCO, NCO, and ZCO reaching extents of 100%, 94%, and 90%, respectively, after 2 h, with correspondingly higher rate constants. The high stability of the samples was observed after the photocatalytic tests.

FIG. 47(a) shows the absorption spectra corresponding to the mixed nanosheet and MB solution as a function of irradiation time. The considerable reduction of the absorption peak in the first 40 min is an indication of rapid chemical breakdown of MB followed by almost diminishing of the peak after 2 h. The kinetics of the photodegradation were explored by plotting ln(Aₜ/A₀), where Aₜ is the dye absorption at time (t) and A₀ is the dye absorbance prior to irradiation, against irradiation time using a pseudo first-order reaction model, as shown in FIG. 47(b). Additionally, FIG. 47(c) illustrates a plot comparing the dye degradation performance of the holey nanosheets synthesised in this work and a selection of best previously-reported performances. The experimental conditions of the work are summarized in Table 4. Further, FCO, with a visible light region bandgap, exhibits a remarkable enhancement in dye degradation performance by almost 100% degradation after 2 h (FIG. 47(d)).

The performance of the nanosheets can be attributed to two mechanisms: 1) High density of structural defects modifying the electronic properties of the nanosheets by narrowing the bandgap. The atomic layer of the nanosheet offers a high surface-to-volume ratio, which considerably enhances the exposed facets at the dye-nanosheet interfacial region. 2) This performance is shown to be improved significantly by fabrication of mixed heterojunction nanostructures that minimize the density of electron/hole recombination, introduces a high number of defects which act as active sites, thereby resulting in high numbers of ROS within the solution to catalyse the dye degradation.

TABLE 4
Comparison of photocatalytic activity (methylene blue degradation) of different nanostructures
of CeO₂ and CeO₂-based materials. Examples as indicated are results from embodiments
of the disclosure, with remaining entries being prior art results.

| | | | Dye | Solids | | % | |
|---|---|---|---|---|---|---|---|
| | | Dimensions | concentration | Loading | Light | Degradation | |
| Material | Morphology | (nm) | (mol L⁻¹) | (mg mL⁻¹) | Source | (1 h) | |
| CeO₂ | Particle | 8 | 5 × 10⁻⁷ | 1 | Sunlight | ~30 | Prior art |
| RGO-CeO₂ | NS-Particle | 8 | 5 × 10⁻⁷ | 1 | Solar light | ~60 | Prior art |
| CeO₂—CuO | Irregular | 9 | 1.5 × 10⁻⁵ | 1 | Sunlight | 17.8 | Prior art |
| CeO₂—V₂O₅ | Irregular | 11 | 1.5 × 10⁻⁵ | 1 | Sunlight | 16.6 | Prior art |
| CeO₂ | Irregular | 13 | 1.5 × 10⁻⁵ | 1 | Sunlight | 1.4 | Prior art |
| CeO₂-RGO | Spherical | ~12 | 0.37 × 10⁻⁶ | 0.8 | Visible light | 30 | Prior art |
| CeO₂₋ₓ | Octahedron | ~22 | 0.60 × 10⁻⁶ | 0.1 | Visible light | 12 | Prior art |
| CeO₂ | Irregular | 11 | 0.5 × 10⁻⁵ | 2 | Solar light | 60 | Prior art |
| CeO₂ | Hollow sphere | 10000 | 2.5 × 10⁻⁵ | 0.52 | Solar light | 60 | Prior art |
| CeO₂ | Irregular | ~25 | N/A | N/A | Solar light | ~48 | Prior art |
| Gd—CeO₂ | Irregular | ~25 | N/A | N/A | Solar light | ~63 | Prior art |
| Sm—CeO₂ | Irregular | ~25 | N/A | N/A | Solar light | ~70 | Prior art |
| CeO₂₋ₓ | Holey 2D | ~1 | 1 × 10⁻⁵ | 0.5 | Solar light | ~70 | Example 2.1/2.2 |
| FCO | Holey 2D | ~1 | 1 × 10⁻⁵ | 0.5 | Solar light | ~90 | Example 2.5 |
| NCO | Holey 2D | ~1 | 1 × 10⁻⁵ | 0.5 | Solar light | ~76 | Example 2.6 |
| ZCO | Holey 2D | ~1 | 1 × 10⁻⁵ | 0.5 | Solar light | ~74 | Example 2.7 |

Further, complete CO oxidation at ˜150° C. by CeO₂₋ₓ2D-3D scaffold and its lowering to ˜90° C. through modification as a CeO₂-based hybrid yields the lowest temperatures yet reported for CO oxidation (see Table 5).

TABLE 5
Comparison of catalytic activity (CO conversion) of different nanostructures
of CeO₂ materials. Examples as indicated are results from embodiments
of the disclosure, with remaining entries being prior art results.

| | | | BET | Pore | | | |
|---|---|---|---|---|---|---|---|
| | | Dimensions | surface area | Volume | | | |
| Material | Morphology | (nm) | (m² g⁻¹) | (cm₃/g) | T₅₀ | T₁₀₀ | |
| CeO₂ | Irregular | ~12 | 56.70 | — | 304 | ~400 | Prior art |
| CeO₂ | rod | d: 20-90 | — | 306 | 400 | 400 | Prior art |
| CeO₂ | Nanowire | 8.1 | 76.9 | 272 | ~350 | ~350 | Prior art |
| CeO₂ | Nanobundles | 9.2 | 130.4 | 0.09 | 213 | 280 | Prior art |
| CeO₂ | rod | — | — | — | — | 280 | Prior art |
| CeO₂ | Irregular | 8-9 | 55.7 | 215 | 260 | | Prior art |
| CeO₂ | Pit-confined | 0.6 | — | 131 | 220 | 220 | Prior art |
| | Nanosheet | | | | | | |
| CeO₂₋ₓ | 2D-3D | 4-5 | 122.9 | 0.3 | 146 | 150 | Example |
| | framework | | | | | | 2.1/2.2 |
| Cu-doped | 2D-3D | 4-5 | 230.8 | 0.4 | 85 | 90 | Example |
| CeO₂₋ₓ | framework | | | | | | |

### Catalytic Performance of Different CeO₂₋ₓ Morphologies

Catalytic performance of the different CeO₂₋ₓ nanostructures was compared by testing their activity in CO oxidation. The results in FIG. 64 show that the CO conversion rates decrease in the order ultrathin sheet>pseudo-octahedron>sphere>leaf. For example, at 400° C., these values are 21.1, 12.8, 1.93 and 0.0 mol g⁻¹ s⁻¹, respectively. The turnover frequency (TOF) values calculated on the basis of the CO molar ratio for each of the catalysts at this temperature show that the ultrathin holey nanosheet (surface area=81 m²·g⁻¹, pore volume=0.32 cm³ g⁻¹) exhibited the highest TOF value of 4.4×10⁻³ mol mol⁻¹ s⁻¹, which is 1.5 times higher than that of the pseudo-octahedron (surface area=47 m²·g⁻¹, pore volume=0.42 cm³ g⁻¹). This value for the nanosheet is also 5 times that of the sphere (surface area=53 m²·g⁻¹, pore volume=0.15 cm³ g⁻¹) and nearly 50 times that of the leaf (surface area=6 m²·g⁻¹, pore volume≈0 cm³ g⁻¹). These results confirm that the combined surface and pore volumes reflect the density of active sites, which consist of unsaturated coordination bonds that enhance CO adsorption. Further, the polycrystalline nature of the nanostructures is important because V_{O}^{••} as point defects have been shown to be present at high concentrations along the grain boundaries.

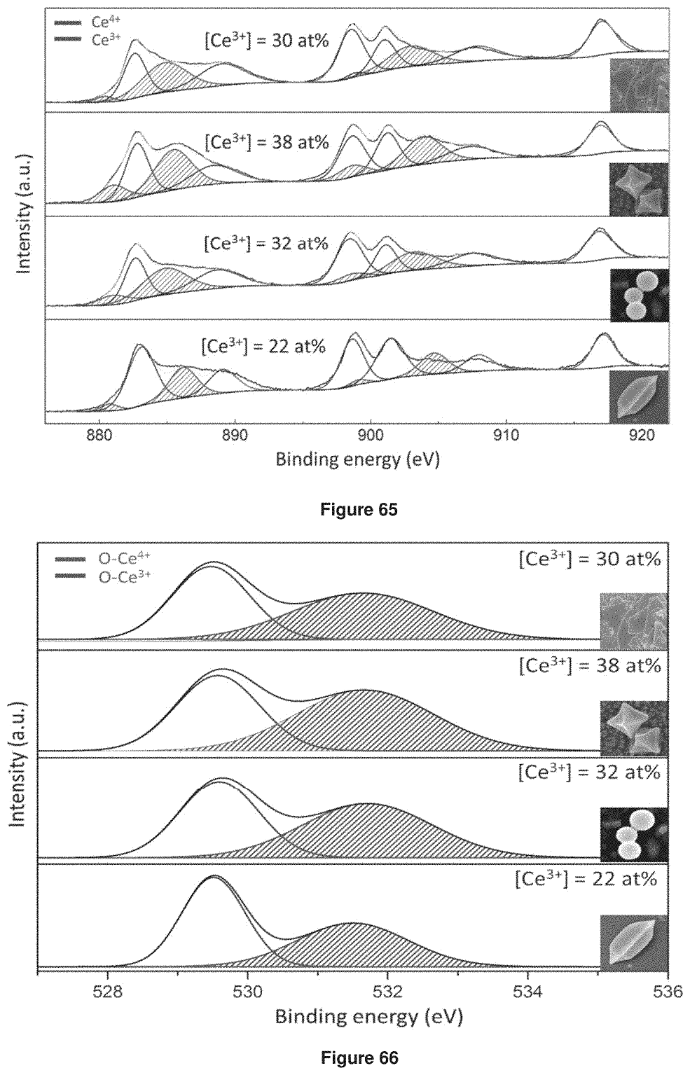

The kinetics of catalysis also were characterised through Arrhenius plots in order to determine the activation energies (Ea) for CO oxidation for the different nanostructures. As expected, these follow in the same relative order as the CO conversion rates and TOF values increase for the ultrathin sheet, pseudo-octahedron, sphere, and leaf: 47<58<115<134 kJ/mol, respectively. It is significant to note that high [V_{O}^{••}] of the CeO₂ samples, obtained from quantitative analysis of XPS results in FIGS. 65, 66, play an important role in the catalytic activity by facilitating CO adsorption and accelerating the mobility of lattice oxygen to enhance the desorption of CO2.

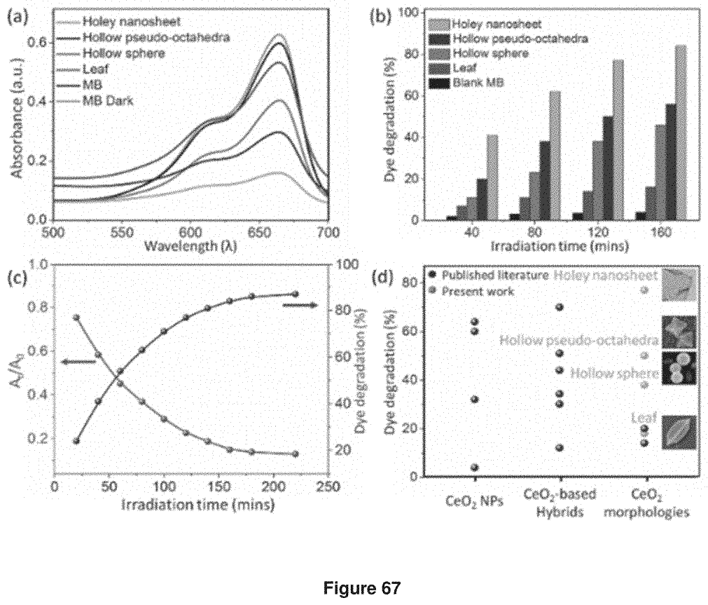

Photocatalytic performance of the CeO₂₋ₓ morphologies prepared herein was investigated by photodegradation of MB during 100 mW/cm² of irradiance at AM 1.5 G solar illumination. The maximal intensities of the absorbance peaks, at 664 cm⁻¹, were used as the bases for the comparative assessment, the data for which are shown in FIG. 67(a). FIG. 67(b) reveals that there are three levels of performance for the dye degradation: high (84% holey nanosheet), medium (55% pseudo-octahedron, 40% sphere), and low (16% leaf). These data are in agreement with the CO oxidation activities, suggesting the predominant roles of surface area and pore volume in catalytic activities.

The kinetics of degradation by the holey nanosheets, plotted in terms of the ratio of absorbance at time t (Aₜ) to the absorbance at the initial time (A₀) against the irradiation time are shown in FIG. 67(c). The rate constant (k) of the degradation was determined to be 0.014 min⁻¹, which can be contrasted with the only other published values obtained under similar test conditions, namely 0.003 min⁻¹ and 0.012 min⁻¹. The observed high efficiency for pure CeO₂₋ₓ is attributed to two principal factors. First, the holey and thin nanostructure provided high accessibility of the charge carriers to the active sites owing to the short diffusion distances from the bulk to the surfaces. This positive characteristics resulted in reduced charge carrier recombination times, as has been reported previously for catalysts, such as holey nanosheets of Ru₃Al^{[42]} and Ni(OH)^{[43]}, that showed enhanced hydrogen and oxygen evolution reactions (HER and OER, respectively). Second, the XPS data reveal the high areal densities of active sites through the high calculated [V_{O}^{••}] values (FIG. 65), which have been determined to be the relevant active sites for reactions.

FIG. 67(d) illustrates a range of published values for photodegradation tests conducted for different pure and hybrid CeO₂₋ₓ morphologies of variable sizes. The superiority of the holey nanosheet morphology is demonstrated by the extent of degradation. Further, analysis reveals that different CeO₂ morphologies with crystallite sizes ≤20 nm exhibited BET specific surface areas in the range of 2-65 m²·g⁻¹, and photodegradation extents in the range of ˜4-70%. These values may be contrasted with those for the holey nanosheet morphology, which exhibited crystallite sizes in the range 4-8 nm, specific surface area 81 m²·g⁻¹, and outstanding performance of 77% photodegradation. The latter is the best performance for CeO₂₋ₓ yet reported. Comparison of the data for the present work highlights the dominance of the effect of the accessible active sites as revealed most distinctly by the coupled specific surface area and pore volume; these are the predictors of performance.

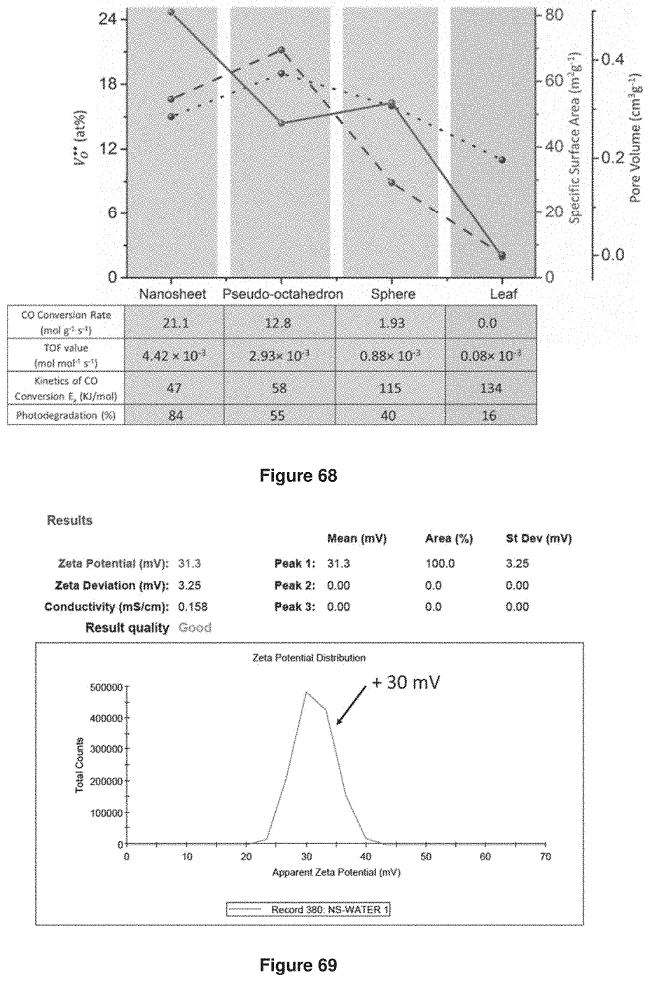

The impact of the architecture, defect equilibria, and nanostructure on the catalytic and photocatalytic performances of CeO₂₋ₓ is summarised in FIG. 68, which plots the oxygen vacancy concentrations ([V_{O}^{••}]), specific surface areas, and pore volumes for the four morphologies. These data are compared to the tabulation of the CO conversion rates, turnover frequencies, required activation energy (Eₐ), and photodegradations. These data showed that the predominant factor controlling the performances is the specific surface area, which reflects the density of active sites. Table 6 provides comparative data for the present work and other equivalent studies, again confirming the predominant effect of the surface area. However, the inconsistent trend for the pseudo-octahedra and spheres shows that this parameter is mitigated by the effects of the oxygen vacancy concentration and the pore volume. Finally, there is no direct correlation between the morphologies and the oxygen vacancy concentrations, which are concentrated at the crystallite and grain boundaries. While such correlations have been observed for single-crystal CeO₂₋ₓ before, the disagreement with the present work highlights the effect of the polycrystalline nature of these architectures.

Overall, holey 2D CeO₂₋ₓ nanostructures showed outstanding photocatalytic performances. These catalytic properties may derive from the short charge carrier diffusion distances and low recombination density that result from the thin, holey, and polycrystalline nanosheets, which contain high concentrations of active sites.

### Example 8: Metal Sulfide Based Nanostructures

The Ce-CP may be used as a precursor to form hybrid CeO₂₋ₓ-based macrolayers with incorporated carbon and sulphur (Ce/S/C).

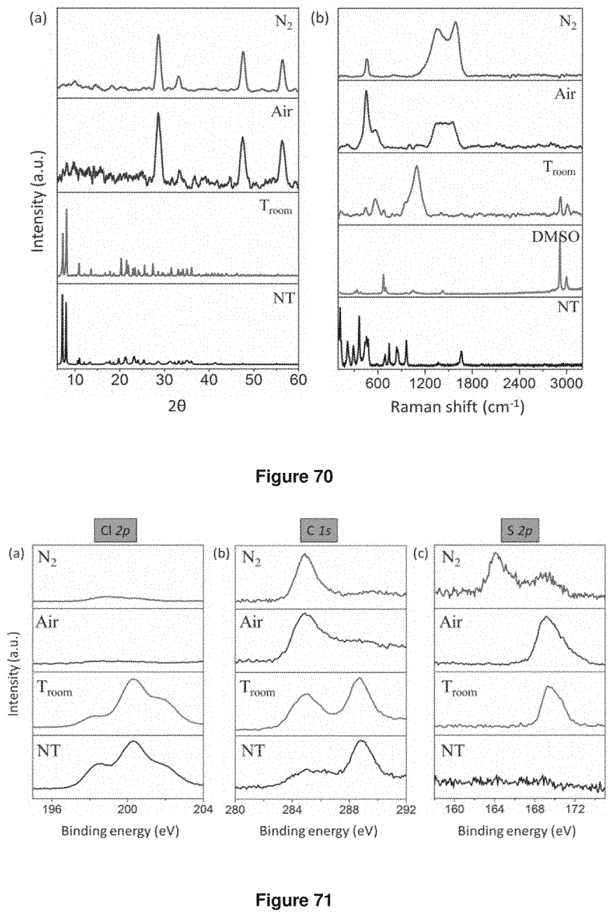

The transformation of Ce-CP into Ce/S/C was investigated structurally using XRD and Raman analyses, the results of which are given in FIG. 70. The XRD data confirms that the disassembly/reassembly of Ce-CP using DMSO solvent retains the triclinic structure of pristine Ce-CP. Annealing the reassembled Ce-CP in air and N₂ atmospheres resulted in oxidation to the CeO₂ fluorite structure. The Raman spectra (FIG. 70b) obtained for Ce-CP and DMSO-derived Ce-CP reveals a predominant peak at 1040 cm⁻¹ attributed to the A_{g} vibrational mode of SO₄ in the Ce sulphate structure. In contrast, the number density of bonds between Ce and the COO⁻ groups of TCA decreased significantly after reassembly, as suggested by the decreasing intensities of the adjacent peaks at 450 cm⁻¹ and 470 cm⁻¹.

Annealing the DMSO-derived Ce-CP in air and N₂ atmospheres yielded CeO₂ with carbon incorporated to the structure. The two peaks at 1300 cm⁻¹ and 1600 cm⁻¹, which are the D-mode and G-mode confirming the presence of graphitic carbon. It is significant to note that the F₂ g mode, which is for stretching vibrations between Ce and O in CeO₂, appeared for both air- and N₂-related spectra. However, this peak shifted to lower value (457 cm⁻¹) for the CeO₂ obtained in air. This can be owing to the presence of E₁ g vibrational mode of SO₄ group in Ce sulphate structure. The Raman spectra for the sample calcined in air also shows a small peak at 1057 cm⁻¹ attributed to the SO₄. These results indicate that the formation of hybrid CeO₂-based carbon and sulphur heterostructures involves a two-step process of reassembly and post-oxidation.

Additional analyses by XPS surface characterisation are shown in FIG. 71. The variations in the Cl concentration are shown in FIG. 71a, where the intensity of Cl 2p orbital is unchanged in the Ce-CP and DMSO-derived Ce-CP while the calcinations in both air and N₂ resulted in near-complete removal. The XPS analyses of the C is orbital in FIG. 71b revealed similar differentials in that the Ce-CP and DMSO-derived Ce-CP exhibited C—O—C bonding (286 eV) and O—C═O bonding (289 eV), the former of which increased by introduction of DMSO in the structure. Calcination of the DMSO-derived Ce-CP resulted in near-elimination of the less stable O═C bonding, although there is a small amount of residual such bonding albeit shifted to higher binding energy owing to increased C—C bond covalency once the highly electronegative Cl groups are removed. The predominant presence of peak at 286 eV can indicate the formation of graphite structures. Calcination in N₂ reveals the higher peak intensity suggesting the formation of higher graphitic carbon concentration, relative to that of obtained in air.

This is confirmed by the XPS analyses of the S 2p orbital shown in FIG. 71c. The use of DMSO solvent led to the formation of Ce sulphate, as confirmed by the S 2p_{3/2} peak at 169 eV. This peak is consistent with those reported for cerium sulphate (Ce(SO₄)₂), where the oxidation state of sulphur is +6. The structure of Ce(SO₄)₂ remained unchanged during calcination in air. However, calcination in N₂ resulted in an appearance a peak centred at lower energy of 164 eV, which is attributed to the sulphur with oxidation states of +4, indicating the formation of CeO₂/SO₂.

The simultaneous reduction of S⁶⁺ to S⁴⁺ and oxidation of Ce³⁺ to Ce⁴⁺ under N₂ suggest the likelihood of IVCT according to the electron exchange reaction:

S⁶⁺+2Ce³⁺→S⁴⁺+2Ce⁴⁺

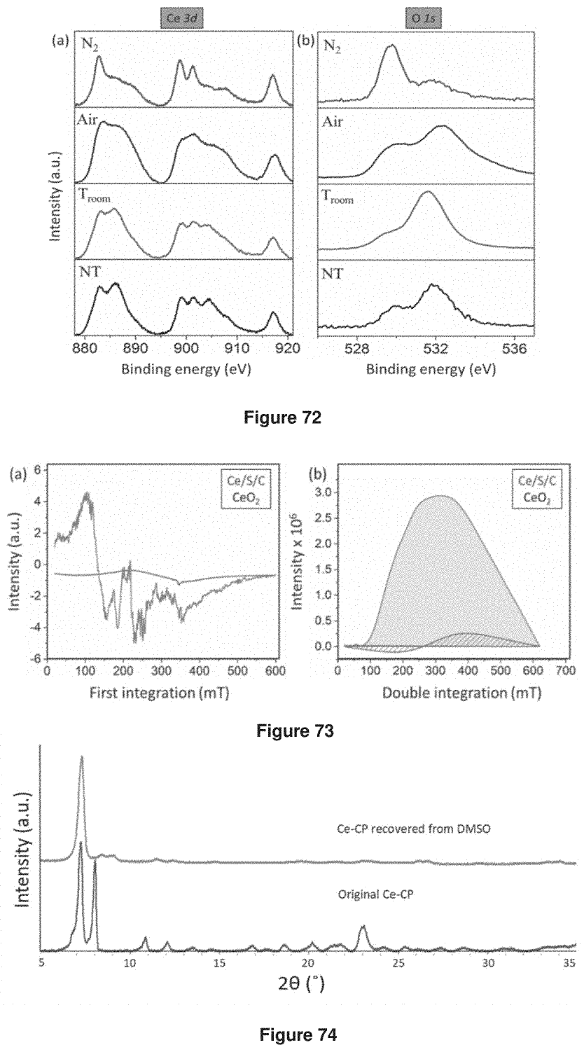

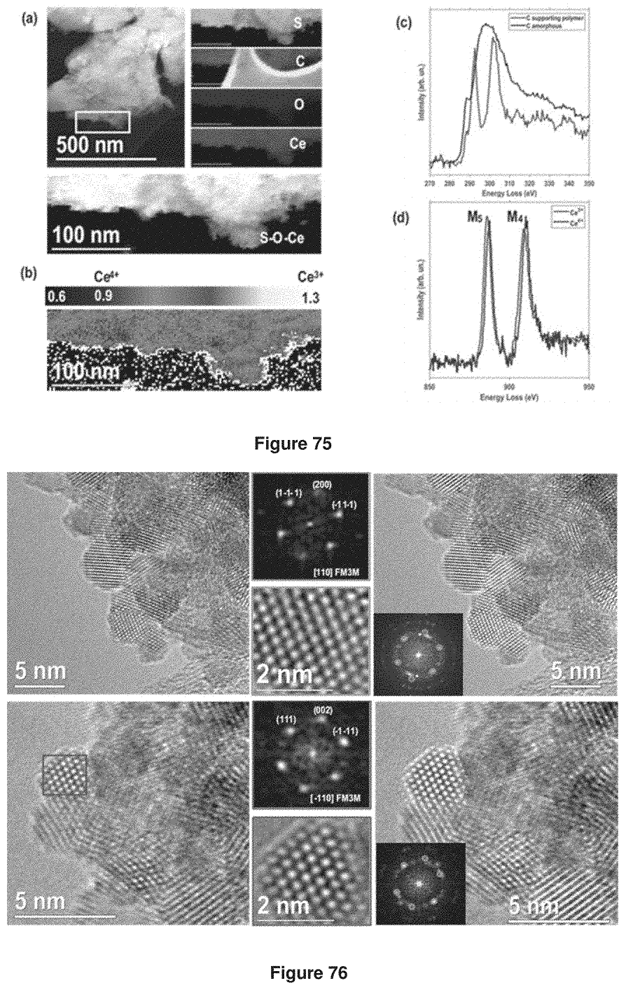

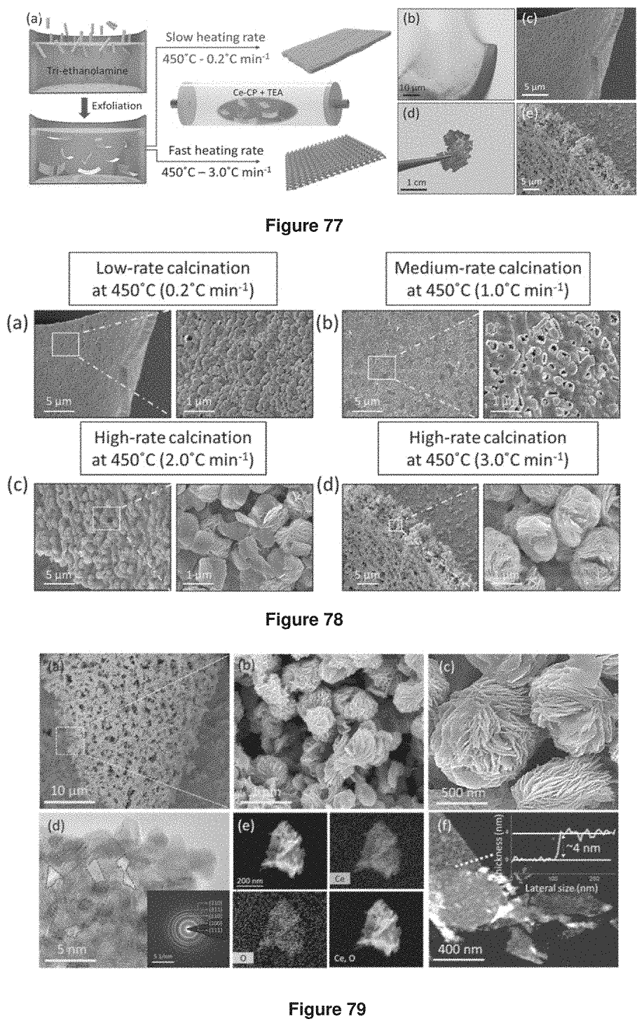

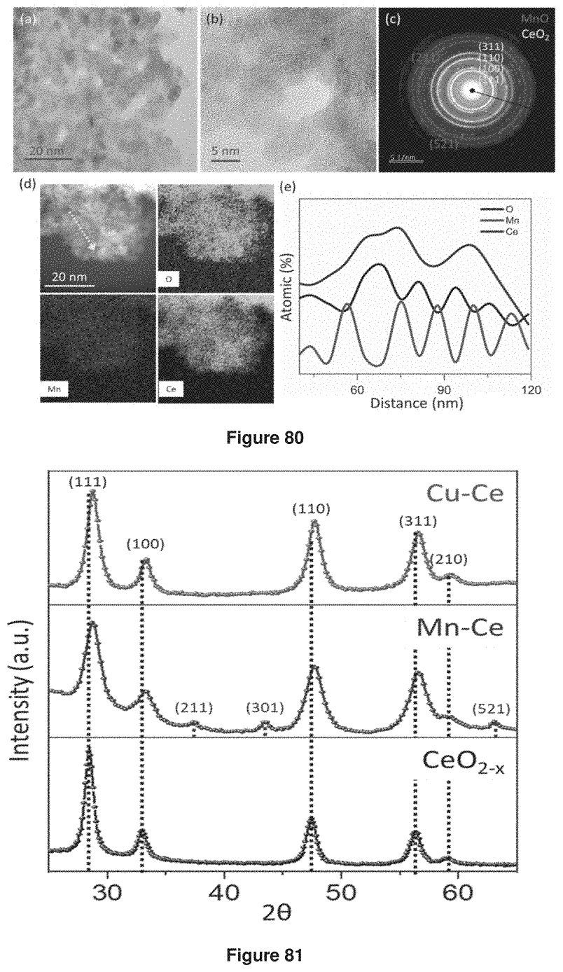

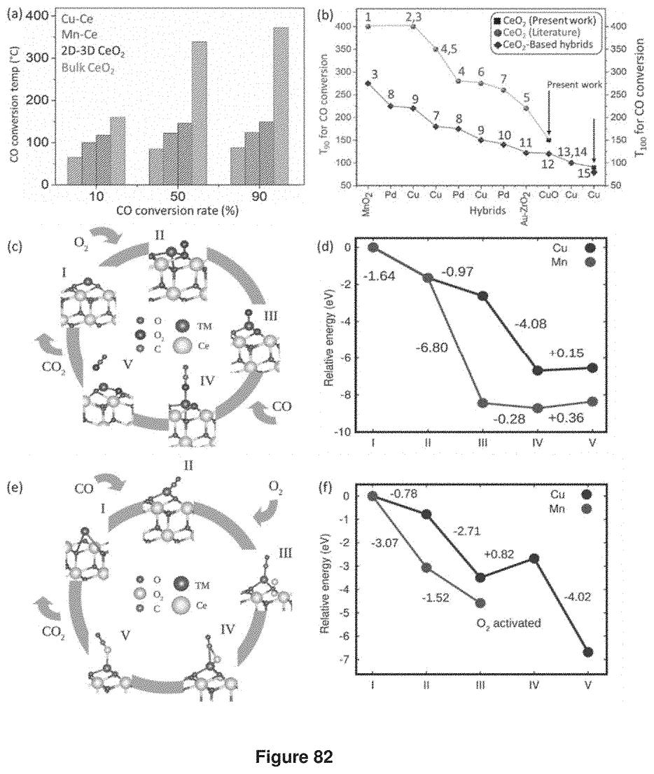

The feasibility of IVCT is confirmed by the XPS data, as shown in FIGS. 72 a, b. However, the corresponding XPS data do not show this reaction to occur after calcination in air. Hence, under N₂, S⁶⁺ to S⁴⁺ reduction is possible and the easy Ce³⁺ to Ce⁴⁺ oxidation facilitates IVCT as a means of charge transfer between the CeO₂ and sulphate structures. In contrast, under air, the absence of the S⁷⁺ valence state effectively precludes S oxidation to S⁸⁺ and so the Ce³⁺ cannot oxidise to Ce⁴⁺ through IVCT; the latter is confirmed in FIG. 72a, b. The preceding results show that calcination N₂ results in the formation of a CeO₂/graphitic oxides/Ce sulphate heterojunction structures.

The concentrations of the resultant structural defects associated with the new heterojunction Ce/S/C and pristine CeO₂ were characterised using EPR, the data for are shown in FIG. 73. The hyperfine pattern in FIG. 73a indicate that, relative to pristine CeO₂, there are several types of defects present in the heterostructure and the area in FIG. 73b shows that there is a very high concentration of total defects.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A layered metal coordination polymer comprising two or more layers, each layer comprising metal atoms coordinated to an organic linker to form a metal coordination polymer layer;
wherein the organic linker is selected from one or more compounds having the structure of Formula (1):
X-R¹ (1)
wherein:
X is a metal binding moiety for coordinative bonding to a metal atom; and
R¹ is an optionally interrupted alkyl, alkenyl or alkynyl group substituted with one or more halogens for forming an electrostatic interaction with an adjacent metal coordination polymer layer to form the layered metal coordination polymer.

2. (canceled)

3. The layered metal coordination polymer of claim 1, wherein the layered metal coordination polymer comprises a plurality of labile ions interspersed between the metal coordination polymer layers that form the electrostatic interaction between the one or more halogens of the organic linker of each metal coordination polymer layer to form the layered metal coordination polymer.

4. (canceled)

5. (canceled)

6. The layered metal coordination polymer of claim 3, wherein the electrostatic interaction between the labile ions interspersed between the metal coordination polymer layers and the one or more halogens of the organic linker of each metal coordination polymer layer is substantially orthogonal to the coordinative bonding between the metal binding moiety (X) and the metal atom within the metal coordination polymer layer.

7.-10. (canceled)

11. The layered metal coordination polymer of claim 1, wherein the one or more halogens are selected from the group consisting of F, Cl, Br and/or I, or one or more halides thereof selected from the group consisting of Li, Na, K, Rb and/or Cs.

12. (canceled)

13. (canceled)

14. The layered metal coordination polymer of claim 1, wherein the metal binding moiety (X) is a monodentate or a bidentate ligand that forms a bridging coordinative bond to two or more metal atoms to form the metal coordination polymer layer, wherein the metal binding moiety (X) comprises a carboxylate, amine, hydroxyl, or thiol.

15. (canceled)

16. (canceled)

17. The layered metal coordination polymer of claim 1, wherein the organic linker is trifluoroacetic acid, trichloroacetic acid, tribromoacetic acid, or triiodoacetic acid.

18. (canceled)

19. The layered metal coordination polymer of claim 1, wherein the metal atom is a metal ion, wherein the metal ion is univalent or multivalent and of one or more metals selected from a rare earth metal, transition metal, Group 13, Group 14 or Group 15 metal of the Periodic Table.

20. (canceled)

21. (canceled)

22. The layered metal coordination polymer of claim 19, wherein the metal ion is selected from Ce³⁺, Ce⁴⁺, Ti⁴⁺, Zr⁴⁺ or Zn²⁺.

23. The layered metal coordination polymer of claim 1, wherein the metal coordination polymer is a cerium metal coordination polymer having the formula Ce(TCA)₂(OH)₂.2H₂O.

24. The layered metal coordination polymer of claim 23, wherein the cerium metal coordination polymer is characterised by an X-ray powder diffraction (XRD) pattern comprising one or more principal peaks located at about 7.2, 8.1, 10.9, 20.6, 22.0, 23.1, and 23.2 degrees 2θ.

25. (canceled)

26. (canceled)

27. A process for preparing a layered metal coordination polymer of claim 1, comprising combining a metal atom source and the organic linker to form the layered metal coordination polymer, wherein the step of combining the metal atom source and the organic linker comprises mixing an aqueous solution comprising the metal atom source and the organic linker to form the layered metal coordination polymer.

28. (canceled)

29. The process of claim 27, wherein the step of forming the layered metal coordination polymer comprises hydrothermal treatment.

30. The process of claim 27, wherein the step of forming the layered metal coordination polymer comprises electrodeposition.

31. The process of claim 30, wherein the initial pH of the aqueous solution during electrodeposition is less than about 7.

32. (canceled)

33. (canceled)

34. The process of claim 30, wherein the electrodeposition is performed within the oxygen evolution region of the aqueous solution comprising the metal atom source and the organic linker.

35.-38. (canceled)

39. The process of claim 27, wherein the layered metal coordination polymer is disassembled in an organic solvent and reassembled by evaporation of the organic solvent, wherein the reassembly from the organic solvent changes the morphology of the metal coordination polymer.

40.-43. (canceled)

44. The process of claim 27, wherein the layered metal coordination polymer is exfoliated to obtain one or more metal coordination polymer layers.

45.-49. (canceled)

50. A method of forming a nanostructure, comprising:
providing a layered metal coordination polymer of claim 1, and
removing at least some of the organic linkers to form the nanostructure.

51.-56. (canceled)

57. The method of claim 50, wherein prior to removing at least some of the organic linkers to form the nanostructure, the layered metal coordination polymer is exfoliated to obtain a dispersion of metal coordination polymer layers.

58.-60. (canceled)

61. The method of claim 50, wherein prior to removing at least some of the organic linkers to form the nanostructure, the metal coordination polymer is disassembled in an organic solvent and reassembled from the organic solvent by evaporation.

62.-67. (canceled)

68. The method of claim 50, wherein the nanostructure is a holey oxide nanostructure, and the step of removing at least some of the organic linkers forms the holey nanostructure.

69. (canceled)

70. The method of claim 50, wherein one or more adsorbate species are adsorbed onto the surface of the nanostructure to form one or more heterojunction nanostructures.

71.-89. (canceled)

90. A catalyst composition comprising the nanostructure prepared using the method of claim 50.

91. (canceled)

92. (canceled)
